SEQUENCE LISTING (135 sequences)

SEQ ID NO: 1 - protein, 5 aa, Mus musculus
TYAMN

SEQ ID NO: 2 - protein, 16 aa, Mus musculus
RSSKSLLHSNGITYLY

SEQ ID NO: 3 - protein, 19 aa, Mus musculus
RIRSKNNYYATYYADSVKD

SEQ ID NO: 4 - protein, 7 aa, Mus musculus
QMSNLAS

SEQ ID NO: 5 - protein, 9 aa, Mus musculus
AQNLELPWT

SEQ ID NO: 6 - protein, 5 aa, Mus musculus
TFGMH

SEQ ID NO: 7 - protein, 10 aa, Mus musculus
SASSSVGYMY

SEQ ID NO: 8 - protein, 17 aa, Mus musculus
YISSGSSSIYYADTVKG

SEQ ID NO: 9 - protein, 7 aa, Mus musculus
LTSILAS

SEQ ID NO: 10 - protein, 12 aa, Mus musculus
SGLGTSPHAMDY

SEQ ID NO: 11 - protein, 9 aa, Mus musculus
QQWISNPLT

SEQ ID NO: 12 - protein, 5 aa, Mus musculus
NYGVH

SEQ ID NO: 13 - protein, 11 aa, Mus musculus
RASQTIGTNIH

SEQ ID NO: 14 - protein, 16 aa, Mus musculus
VIWTGGNTEYNSALMS

SEQ ID NO: 15 - protein, 7 aa, Mus musculus
YASESIS

SEQ ID NO: 16 - protein, 12 aa, Mus musculus
DDYYFGGGAMDY

SEQ ID NO: 17 - protein, 9 aa, Mus musculus
QQSNNWPLT

SEQ ID NO: 18 - protein, 5 aa, Mus musculus
SFTLY

SEQ ID NO: 19 - protein, 15 aa, Mus musculus
RASESVDNYGNSFMH

SEQ ID NO: 20 - protein, 17 aa, Mus musculus
YIDPSNGGTTYNQRFKG

SEQ ID NO: 21 - protein, 7 aa, Mus musculus
LASNLES

SEQ ID NO: 22 - protein, 14 aa, Mus musculus
GIYDGYYVGKIFDY

SEQ ID NO: 23 - protein, 9 aa, Mus musculus
QQNNEDPPT

SEQ ID NO: 24 - protein, 5 aa, Mus musculus
SYWMD

SEQ ID NO: 25 - protein, 15 aa, Mus musculus
RASESIDSYGNSFMH

SEQ ID NO: 26 - protein, 17 aa, Mus musculus
EIYPSNGRTKYNEKFKN

SEQ ID NO: 27 - protein, 7 aa, Mus musculus
LASNLES

SEQ ID NO: 28 - protein, 14 aa, Mus musculus
GGYDVYYVGNTLDY

SEQ ID NO: 29 - protein, 9 aa, Mus musculus
LQNNGDPWT

SEQ ID NO: 30 - protein, 5 aa, Mus musculus
SYWMH

SEQ ID NO: 31 - protein, 17 aa, Mus musculus
KSSQSLLYSSNQKNYLA

SEQ ID NO: 32 - protein, 17 aa, Mus musculus
AIYPGNSDTSYNQKFKG

SEQ ID NO: 33 - protein, 7 aa, Mus musculus
WASTRES

SEQ ID NO: 34 - protein, 9 aa, Mus musculus
YGNYGYFDV

SEQ ID NO: 35 - protein, 10 aa, Mus musculus
QQYYSYPPYT

SEQ ID NO: 36 - protein, 5 aa, Mus musculus
DDVIN

SEQ ID NO: 37 - protein, 15 aa, Mus musculus
RASKSVSPSDYSYIH

SEQ ID NO: 38 - protein, 17 aa, Mus musculus
EIYPRIGSMYYNENFKG

SEQ ID NO: 39 - protein, 7 aa, Mus musculus
LASNLES

SEQ ID NO: 40 - protein, 11 aa, Mus musculus
YLLTKGYGMDY

SEQ ID NO: 41 - protein, 9 aa, Mus musculus
QHSRELPRT

SEQ ID NO: 42 - protein, 5 aa, Mus musculus
SYGLH

SEQ ID NO: 43 - protein, 10 aa, Mus musculus
RASSSVSYIY

SEQ ID NO: 44 - protein, 16 aa, Mus musculus
FIWSGGATDYNAAFIS

SEQ ID NO: 45 - protein, 7 aa, Mus musculus
LTSDLAS

SEQ ID NO: 46 - protein, 11 aa, Mus musculus
RDDNYALAMDY

SEQ ID NO: 47 - protein, 9 aa, Mus musculus
QQWNSNPLT

SEQ ID NO: 48 - protein, 133 aa, Mus musculus
MLLGLKWVFFVVFYQGVHCEVKLVESGGGLVQPKGSLKLSCAASGFTFNTYAMNWIRQAPGKGLEWVARIRSKNNYYATYYADSVKDRFTISRDDSQSMLYLQMNNLKTEDTAMYYCVSFAYWGQGTLVTVSA

SEQ ID NO: 49 - protein, 132 aa, Mus musculus
MRFSAQLLGLLVLWIPGSTAEIVMTQAAFSNPVTLGTSASISCRSSKSLLHSNGITYLYWYLQKPGQSPRFLIYQMSNLASGVPDRFTSSGSGTDFTLRISRVEAEDVGVYYCAQNLELPWTFGGGTKLEIK

SEQ ID NO: 50 - protein, 140 aa, Mus musculus
MDSRLNLVFLVLILKGVQCDVQLVESGGGLVQPGGSRKLSCAASGFTFSTFGMHWVRQAPEKGLEWVAYISSGSSSIYYADTVKGRFTISRDNPVNTLFLQMTSLRSEDTAMYYCARSGLGTSPHAMDYWGQGTSVTVSS

SEQ ID NO: 51 - protein, 128 aa, Mus musculus
MDFQVQIFSFLLMSASVIMSRGQIVLTQSPTLMSASPGEKVTMTCSASSSVGYMYWYQQKPRSSPKPWIYLTSILASGVPARFSGSGSGTSYSLTISSMEAEDAATYYCQQWISNPLTFGAGTKLELK

SEQ ID NO: 52 - protein, 139 aa, Mus musculus
MAVLVLFLCLAAFPSYVLSQVQMEQSGPGLVAPSQSLSITCTVSDFSLINYGVHWVRQSPGKGLEWLGVIWTGGNTEYNSALMSRLSITKDNSKSQVFLKMNSLQADDTAMYYCARDDYYFGGGAMDYWGQGTSVTVSS

SEQ ID NO: 53 - protein, 127 aa, Mus musculus
MVSTPQFLVFLLFWIPASRGDILLTQSPAILSVSPGERVSFACRASQTIGTNIHWYQQRTNGSPRLLIKYASESISEIPSRFSGSGSGTDFILTISSVESEDIADYYCQQSNNWPLTFGAGTKLELR

SEQ ID NO: 54 - protein, 142 aa, Mus musculus
MEWRWIFLLLLSGTTGVHSEIQLQQSGPELVKPGASVKVSCKASGYTFTSFTLYWVKQSHGKSLEWIGYIDPSNGGTTYNQRFKGKATLTVDKSSTTAYMHLNSLTSEDSSVYYCARGIYDGYYVGKIFDYWGQGTTLTVSS

SEQ ID NO: 55 - protein, 131 aa, Mus musculus
METDTLLLWVLLLWVPGSTGNIVLTQSPTSLAVSLGQRATISCRASESVDNYGNSFMHWYQQKPGQPPRLLIYLASNLESGVPARFSGSGSRTDFTLTIDPVEADDAATYYCQQNNEDPPTFGGGTKLEIR

SEQ ID NO: 56 - protein, 142 aa, Mus musculus
MGWSYIILFLVATATDVHSQVQLQQPGAELVKPGASVKLSCKASGYTFTSYWMDWVKQRPGQGLEWIGEIYPSNGRTKYNEKFKNKATLTVDNSSRTAYMHLSSLTSEDSAVYYCARGGYDVYYVGNTLDYWGQGTSVTVSS

SEQ ID NO: 57 - protein, 131 aa, Mus musculus
METDTLLLWVLLLWVPGSTGNIVLTQSPASLAVSLGQRATISCRASESIDSYGNSFMHWYHQKPGQPPKLLIYLASNLESGVPARFSGSGSRTDFTLTIDPVEADDAATYYCLQNNGDPWTFGGGTRLEIK

SEQ ID NO: 58 - protein, 137 aa, Mus musculus
MECNWILPFILSVTSGVYSEVQLQQSGTVLARPGASVKMSCKASGYTFTSYWMHWVKQRPGQGLEWIGAIYPGNSDTSYNQKFKGKAKLTAVTSTSTAYMELSSLTNEDSAVYYCTIYGNYGYFDVWGAGTTVTVSS

SEQ ID NO: 59 - protein, 134 aa, Mus musculus
MDSQAQVLMLLLLWVSGTCGDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPPYTFGGGTKLEIK

SEQ ID NO: 60 - protein, 138 aa, Mus musculus
MEWRIFLFILSGTACVHSQDQLLQSGPELVKPGTSVKMSCRASGYTFSDDVINWVRKRTGQGLEWIGEIYPRIGSMYYNENFKGRATLTADKSSNTVYIHLSSLTSEDSAVYFCARYLLTKGYGMDYWGQGTSVTVSS

SEQ ID NO: 61 - protein, 131 aa, Mus musculus
METDTLLLWVLLLWVPGSTGDIVLTQSPASLAVSLGQRATISCRASKSVSPSDYSYIHWYQQKPGQPPKLLIYLASNLESGVPARFSGSGSGTDFTLNIHPVEEEDAATYYCQHSRELPRTFGGGTKLEIK

SEQ ID NO: 62 - protein, 138 aa, Mus musculus
MAVLGLLFCLVTFPSCVLSQVQLKQSGPGLVQPSQSLSITCTVSGFSLTSYGLHWVRQSPGKGLEWLGFIWSGGATDYNAAFISRLSISKDNSKRQVFFKMNSLQANDTAIYFCARRDDNYALAMDYWGQGTSVTVSS

SEQ ID NO: 63 - protein, 128 aa, Mus musculus
MDFQVQIFSFLLMSASVIMSRGQIVLTQSPALMSASPGEKVTMTCRASSSVSYIYWYQQKPRSSPKPWIYLTSDLASGVPTRFSGSGSGTSYFLTISSMEAEDTATYYCQQWNSNPLTFGAGTKLELK

SEQ ID NO: 64 - DNA, 399 bp, Mus musculus
  source 1..399: mol_type = genomic DNA
atgctgttggggctgaagtgggttttctttgttgttttttatcaaggtgtgcattgtgaggtaaaacttgttgagtctggtggaggattggtgcagcctaaagggtcattgaaactctcatgtgcagcctctggattcaccttcaatacctacgccatgaactggatccgccaggctccaggaaagggtttggaatgggttgctcgcataagaagtaaaaataattattatgcaacatattatgccgattcagtgaaagacaggttcaccatctccagagatgattcacaaagtatgctctatctgcaaatgaacaacttgaaaactgaggacacagccatgtattactgtgtgagctttgcttactggggccaagggactctggtcactgtctctgca

SEQ ID NO: 65 - DNA, 396 bp, Mus musculus
  source 1..396: mol_type = genomic DNA
atgaggttctctgctcagcttctggggctgcttgtgctctggatccctggatccactgcagaaattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcggttcctgatttatcagatgtccaaccttgcctcaggagtcccagacaggttcactagcagtggttcaggaactgatttcacactgagaatcagcagagtggaggctgaggatgtgggtgtttattactgtgctcaaaatctagaacttccgtggacgttcggtggaggcaccaagctggaaatcaaa

SEQ ID NO: 66 - DNA, 420 bp, Mus musculus
  source 1..420: mol_type = genomic DNA
atggactccaggctcaatttagttttccttgtccttattttaaaaggtgtccagtgtgatgtgcagctggtggagtctgggggaggcttagtgcagcctggagggtcccggaaactctcctgtgcagcctctggattcactttcagcacctttggaatgcactgggttcgtcaggctccagagaaggggctggagtgggtcgcatacatcagtagtggcagtagttccatctactatgcagacacagtgaagggccgattcaccatttccagagacaatcccgtgaacaccctgttcctgcaaatgaccagtctaaggtctgaagacacggccatgtattactgtgcaagatccggactcggtactagcccccatgctatggactactggggtcaaggaacctcagtcaccgtctcctca

SEQ ID NO: 67 - DNA, 384 bp, Mus musculus
  source 1..384: mol_type = genomic DNA
atggattttcaagtgcagattttcagcttcctgctaatgagtgcctcagtcataatgtccaggggacaaattgttctcacccagtctccaacactcatgtctgcatctccaggggagaaggtcaccatgacctgcagtgccagctcaagtgtaggttacatgtactggtaccagcagaagccaagatcctcccccaaaccctggatttatctcacatccatcctggcttctggagtccctgctcgcttcagtggcagtgggtctgggacctcttactctctcacaatcagcagcatggaggctgaagatgctgccacttattactgccagcagtggattagtaacccgctcacgttcggtgctgggaccaagctggagctgaaa

SEQ ID NO: 68 - DNA, 417 bp, Mus musculus
  source 1..417: mol_type = genomic DNA
atggctgtcctggtgctgttcctctgcctggctgcatttccaagctatgtcctgtcccaggtgcagatggagcagtcaggacctggcctggtggcgccctcacagagcctgtccatcacttgcactgtctctgacttttcactaattaactatggtgttcactgggttcgccagtctccaggaaagggtctggagtggctgggagtcatttggactggtggaaatacagaatataattcggctctcatgtccagattgagcatcaccaaagacaactccaagagccaagttttcttaaaaatgaacagtctgcaagctgatgacacagccatgtactactgtgcccgagatgactattacttcggtggtggggctatggactactggggtcaaggaacctcagtcaccgtctcctca

SEQ ID NO: 69 - DNA, 381 bp, Mus musculus
  source 1..381: mol_type = genomic DNA
atggtatccacacctcagttccttgtatttttgcttttctggattccagcctccagaggcgacatcttgttgactcagtctccagccatcctgtctgtgagtccaggagaaagagtcagtttcgcctgcagggccagtcagaccattggcacaaacatacactggtatcaacaacgaacaaatggttctccaaggcttctcataaaatatgcttctgagtctatctctgagatcccttccaggtttagtggcagtggatcggggacagattttattctcaccatcagcagtgtggagtctgaagatattgcagattattactgtcaacaaagcaataactggccgctcacgttcggtgctgggaccaagctggagctgaga

SEQ ID NO: 70 - DNA, 426 bp, Mus musculus
  source 1..426: mol_type = genomic DNA
atggaatggagatggatctttctcctcctcctgtcaggaactacaggtgtccactctgagatccagctgcagcagtctggacctgagctggtgaagcctggggcttcagtgaaggtttcctgcaaggcttctggttatacattcactagcttcaccttgtactgggtgaagcagagccatggaaagagccttgagtggattggatatattgatccttccaatggtggtactacctacaaccagaggttcaagggcaaggccacattgactgttgacaagtcctccaccacagcctacatgcatctcaacagcctgacatctgaggactcttcagtctattactgtgcaagaggaatttatgatggttactacgtagggaaaatttttgactactggggccagggcaccactctcacagtctcctca

SEQ ID NO: 71 - DNA, 393 bp, Mus musculus
  source 1..393: mol_type = genomic DNA
atggagacagacacactcctgctatgggtgctgctgctctgggttccaggttccacaggtaacattgtactgacccaatctccaacttctttggctgtgtctctcgggcagagggccaccatatcctgcagagccagtgaaagtgttgataattatggcaatagttttatgcactggtaccagcagaaaccaggacagccacccagactcctcatctatcttgcgtccaacctagaatctggggtccctgccaggttcagtggcagtgggtctaggacagacttcaccctcaccattgatcctgtggaggctgatgatgctgcaacctattactgtcagcaaaataatgaggatcctccgacgttcggtggaggcaccaagctggaaatcaga

SEQ ID NO: 72 - DNA, 426 bp, Mus musculus
  source 1..426: mol_type = genomic DNA
atgggatggagctacatcatcctctttttggtagcaacagctacagatgtccactcccaggtccaactgcagcagcctggggctgaactggtgaagcctggggcttcagtgaagctgtcctgtaaggcttctggctacaccttcaccagttattggatggactgggtgaagcagaggcctggacaaggccttgagtggattggagagatttatcctagcaacggtcgtactaagtataatgagaagttcaagaacaaggccacactgactgtagacaattcctccaggacagcctacatgcatctaagcagcctgacatctgaggactctgcggtctattactgtgcaagagggggctatgatgtttactacgtcggcaatactttggactactggggtcaaggaacctcagtcaccgtctcctca

SEQ ID NO: 73 - DNA, 393 bp, Mus musculus
  source 1..393: mol_type = genomic DNA
atggagacagacacactcctgctatgggtgctgctgctctgggttccaggttccacaggtaacattgtgttgacccaatctccagcttctttggctgtgtctctggggcagagggccaccatatcctgcagagccagtgaaagtattgatagttatggcaatagttttatgcactggtaccaccagaaaccaggacagccacccaaactcctcatttatcttgcatccaacctagaatctggggtccctgccaggttcagtggcagtgggtctaggacagacttcaccctcaccattgatcctgtggaggctgatgatgctgccacctattactgtctccaaaataatggggatccctggacgttcggtggaggcacccggctggagatcaaa

SEQ ID NO: 74 - DNA, 411 bp, Mus musculus
  source 1..411: mol_type = genomic DNA
atggaatgtaactggatacttccttttattctgtcggtaacttcaggggtctactcagaggttcagctccagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtctattactgtacaatctatggtaactacgggtacttcgatgtctggggcgcagggaccacggtcaccgtctcctca

SEQ ID NO: 75 - DNA, 402 bp, Mus musculus
  source 1..402: mol_type = genomic DNA
atggattcacaggcccaggttcttatgttactgctgctatgggtatctggtacctgtggggacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctccgtacacgttcggaggggggaccaagctggaaataaaa

SEQ ID NO: 76 - DNA, 414 bp, Mus musculus
  source 1..414: mol_type = genomic DNA
atggaatggaggatctttctcttcatcctgtcaggaactgcatgtgtccactcccaggatcagctgctgcagtctgggcctgaactggtgaagcctgggacttcagtgaagatgtcctgcagggcttctggatacacattcagtgacgatgtcataaactgggtgcggaagagaactggacagggcctggaatggattggagagatttatcctagaattggtagtatgtactacaatgagaatttcaagggcagggccactctgactgcagacaaatcgtccaacacagtctacattcatctcagcagcctgacatctgaggactctgcggtctatttctgcgcacgatatctactaacgaagggctatggtatggactactggggtcaaggcacctcagtcaccgtctcctca

SEQ ID NO: 77 - DNA, 393 bp, Mus musculus
  source 1..393: mol_type = genomic DNA
atggagacagacacactcctgttatgggtactgctgctctgggttccaggttccactggtgacattgtgctgacacagtctcctgcttccttagctgtgtctctggggcagagggccaccatctcatgcagggccagcaaaagtgtcagtccatctgactatagttatatccactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcaacacagtagggagcttcctcggacgttcggtggaggcaccaagttggaaatcaaa

SEQ ID NO: 78 - DNA, 414 bp, Mus musculus
  source 1..414: mol_type = genomic DNA
atggctgtcttggggctgctcttctgcctggtgacattcccaagctgtgtcctatcccaggtgcagctgaaacagtcaggacctggcctagtgcagccctcacagagcctgtccatcacctgcaccgtctctggtttctcattaactagctatggtttacactgggttcgccagtctccaggaaagggtctggagtggttgggattcatttggagtggtggagccacagactataatgcagctttcatatccagactgagcatcagcaaggacaattccaagaggcaagttttctttaaaatgaacagtctgcaagctaatgacacagccatatatttctgtgccagaagggatgataattacgccttggctatggactactggggtcaaggaacctcagtcaccgtctcctca

SEQ ID NO: 79 - DNA, 384 bp, Mus musculus
  source 1..384: mol_type = genomic DNA
atggattttcaagtgcagattttcagcttcctgctaatgagtgcctcagtcataatgtccaggggacaaattgttctcacccagtctccagcactcatgtctgcatctccaggggagaaggtcaccatgacctgccgtgccagctcaagtgtaagttacatatactggtaccagcagaagccaagatcctcccccaaaccctggatttatctcacatccgacctggcttctggagtccctactcgcttcagtggcagtgggtctgggacctcttactttcttacaatcagtagcatggaggctgaagatactgccacttattactgccagcagtggaatagtaatccgctcacattcggtgctgggaccaagctggaactgaaa

SEQ ID NO: 80 - DNA, 15 bp, Mus musculus
  source 1..15: mol_type = genomic DNA
acctacgccatgaac

SEQ ID NO: 81 - DNA, 48 bp, Mus musculus
  source 1..48: mol_type = genomic DNA
aggtctagtaagagtctcctacatagtaatggcatcacttatttgtat

SEQ ID NO: 82 - DNA, 57 bp, Mus musculus
  source 1..57: mol_type = genomic DNA
cgcataagaagtaaaaataattattatgcaacatattatgccgattcagtgaaagac

SEQ ID NO: 83 - DNA, 21 bp, Mus musculus
  source 1..21: mol_type = genomic DNA
cagatgtccaaccttgcctca

SEQ ID NO: 84 - DNA, 27 bp, Mus musculus
  source 1..27: mol_type = genomic DNA
gctcaaaatctagaacttccgtggacg

SEQ ID NO: 85 - DNA, 15 bp, Mus musculus
  source 1..15: mol_type = genomic DNA
acctttggaatgcac

SEQ ID NO: 86 - DNA, 30 bp, Mus musculus
  source 1..30: mol_type = genomic DNA
agtgccagctcaagtgtaggttacatgtac

SEQ ID NO: 87 - DNA, 51 bp, Mus musculus
  source 1..51: mol_type = genomic DNA
tacatcagtagtggcagtagttccatctactatgcagacacagtgaagggc

SEQ ID NO: 88 - DNA, 21 bp, Mus musculus
  source 1..21: mol_type = genomic DNA
ctcacatccatcctggcttct

SEQ ID NO: 89 - DNA, 36 bp, Mus musculus
  source 1..36: mol_type = genomic DNA
tccggactcggtactagcccccatgctatggactac

SEQ ID NO: 90 - DNA, 27 bp, Mus musculus
  source 1..27: mol_type = genomic DNA
cagcagtggattagtaacccgctcacg

SEQ ID NO: 91 - DNA, 15 bp, Mus musculus
  source 1..15: mol_type = genomic DNA
aactatggtgttcac

SEQ ID NO: 92 - DNA, 33 bp, Mus musculus
  source 1..33: mol_type = genomic DNA
agggccagtcagaccattggcacaaacatacac

SEQ ID NO: 93 - DNA, 48 bp, Mus musculus
  source 1..48: mol_type = genomic DNA
gtcatttggactggtggaaatacagaatataattcggctctcatgtcc

SEQ ID NO: 94 - DNA, 21 bp, Mus musculus
  source 1..21: mol_type = genomic DNA
tatgcttctgagtctatctct

SEQ ID NO: 95 - DNA, 36 bp, Mus musculus
  source 1..36: mol_type = genomic DNA
gatgactattacttcggtggtggggctatggactac

SEQ ID NO: 96 - DNA, 27 bp, Mus musculus
  source 1..27: mol_type = genomic DNA
caacaaagcaataactggccgctcacg

SEQ ID NO: 97 - DNA, 15 bp, Mus musculus
  source 1..15: mol_type = genomic DNA
agcttcaccttgtac

SEQ ID NO: 98 - DNA, 45 bp, Mus musculus
  source 1..45: mol_type = genomic DNA
agagccagtgaaagtgttgataattatggcaatagttttatgcac

SEQ ID NO: 99 - DNA, 51 bp, Mus musculus
  source 1..51: mol_type = genomic DNA
tatattgatccttccaatggtggtactacctacaaccagaggttcaagggc

SEQ ID NO: 100 - DNA, 21 bp, Mus musculus
  source 1..21: mol_type = genomic DNA
cttgcgtccaacctagaatct

SEQ ID NO: 101 - DNA, 42 bp, Mus musculus
  source 1..42: mol_type = genomic DNA
ggaatttatgatggttactacgtagggaaaatttttgactac

SEQ ID NO: 102 - DNA, 27 bp, Mus musculus
  source 1..27: mol_type = genomic DNA
cagcaaaataatgaggatcctccgacg

SEQ ID NO: 103 - DNA, 15 bp, Mus musculus
  source 1..15: mol_type = genomic DNA
agttattggatggac

SEQ ID NO: 104 - DNA, 45 bp, Mus musculus
  source 1..45: mol_type = genomic DNA
agagccagtgaaagtattgatagttatggcaatagttttatgcac

SEQ ID NO: 105 - DNA, 51 bp, Mus musculus
  source 1..51: mol_type = genomic DNA
gagatttatcctagcaacggtcgtactaagtataatgagaagttcaagaac

SEQ ID NO: 106 - DNA, 21 bp, Mus musculus
  source 1..21: mol_type = genomic DNA
cttgcatccaacctagaatct

SEQ ID NO: 107 - DNA, 42 bp, Mus musculus
  source 1..42: mol_type = genomic DNA
gggggctatgatgtttactacgtcggcaatactttggactac

SEQ ID NO: 108 - DNA, 27 bp, Mus musculus
  source 1..27: mol_type = genomic DNA
ctccaaaataatggggatccctggacg

SEQ ID NO: 109 - DNA, 15 bp, Mus musculus
  source 1..15: mol_type = genomic DNA
agctactggatgcac

SEQ ID NO: 110 - DNA, 51 bp, Mus musculus
  source 1..51: mol_type = genomic DNA
aagtccagtcagagccttttatatagtagcaatcaaaagaactacttggcc

SEQ ID NO: 111 - DNA, 51 bp, Mus musculus
  source 1..51: mol_type = genomic DNA
gctatttatcctggaaatagtgatactagctacaaccagaagttcaagggc

SEQ ID NO: 112 - DNA, 21 bp, Mus musculus
  source 1..21: mol_type = genomic DNA
tgggcatccactagggaatct

SEQ ID NO: 113 - DNA, 27 bp, Mus musculus
  source 1..27: mol_type = genomic DNA
tatggtaactacgggtacttcgatgtc

SEQ ID NO: 114 - DNA, 30 bp, Mus musculus
  source 1..30: mol_type = genomic DNA
cagcaatattatagctatcctccgtacacg

SEQ ID NO: 115 - DNA, 15 bp, Mus musculus
  source 1..15: mol_type = genomic DNA
gacgatgtcataaac

SEQ ID NO: 116 - DNA, 45 bp, Mus musculus
  source 1..45: mol_type = genomic DNA
agggccagcaaaagtgtcagtccatctgactatagttatatccac

SEQ ID NO: 117 - DNA, 51 bp, Mus musculus
  source 1..51: mol_type = genomic DNA
gagatttatcctagaattggtagtatgtactacaatgagaatttcaagggc

SEQ ID NO: 118 - DNA, 21 bp, Mus musculus
  source 1..21: mol_type = genomic DNA
cttgcatccaacctagaatct

SEQ ID NO: 119 - DNA, 33 bp, Mus musculus
  source 1..33: mol_type = genomic DNA
tatctactaacgaagggctatggtatggactac

SEQ ID NO: 120 - DNA, 27 bp, Mus musculus
  source 1..27: mol_type = genomic DNA
caacacagtagggagcttcctcggacg

SEQ ID NO: 121 - DNA, 15 bp, Mus musculus
  source 1..15: mol_type = genomic DNA
agctatggtttacac

SEQ ID NO: 122 - DNA, 30 bp, Mus musculus
  source 1..30: mol_type = genomic DNA
cgtgccagctcaagtgtaagttacatatac

SEQ ID NO: 123 - DNA, 48 bp, Mus musculus
  source 1..48: mol_type = genomic DNA
ttcatttggagtggtggagccacagactataatgcagctttcatatcc

SEQ ID NO: 124 - DNA, 21 bp, Mus musculus
  source 1..21: mol_type = genomic DNA
ctcacatccgacctggcttct

SEQ ID NO: 125 - DNA, 33 bp, Mus musculus
  source 1..33: mol_type = genomic DNA
agggatgataattacgccttggctatggactac

SEQ ID NO: 126 - DNA, 27 bp, Mus musculus
  source 1..27: mol_type = genomic DNA
cagcagtggaatagtaatccgctcaca

SEQ ID NO: 127 - protein, 15 aa, Mus musculus
GGGGSGGGGSGGGGS

SEQ ID NO: 128 - protein, 260 aa, Mus musculus
QVQMEQSGPGLVAPSQSLSITCTVSDFSLINYGVHWVRQSPGKGLEWLGVIWTGGNTEYNSALMSRLSITKDNSKSQVFLKMNSLQADDTAMYYCARDDYYFGGGAMDYWGQGTSVTVSSGGGGSGGGGSGGGGSDILLTQSPAILSVSPGERVSFACRASQTIGTNIHWYQQRTNGSPRLLIKYASESISEIPSRFSGSGSGTDFILTISSVESEDIADYYCQQSNNWPLTFGAGTKLELRAAAYPYDVPDYAHHHHHH

SEQ ID NO: 129 - protein, 282 aa, Mus musculus
ATMETDTLLLWVLLLWVPGSTGQVQMEQSGPGLVAPSQSLSITCTVSDFSLINYGVHWVRQSPGKGLEWLGVIWTGGNTEYNSALMSRLSITKDNSKSQVFLKMNSLQADDTAMYYCARDDYYFGGGAMDYWGQGTSVTVSSGGGGSGGGGSGGGGSDILLTQSPAILSVSPGERVSFACRASQTIGTNIHWYQQRTNGSPRLLIKYASESISEIPSRFSGSGSGTDFILTISSVESEDIADYYCQQSNNWPLTFGAGTKLELRAAAYPYDVPDYAHHHHHH

SEQ ID NO: 130 - protein, 22 aa, Mus musculus
ATMETDTLLLWVLLLWVPGSTG

SEQ ID NO: 131 - protein, 431 aa, Mus musculus
QVQMEQSGPGLVAPSQSLSITCTVSDFSLINYGVHWVRQSPGKGLEWLGVIWTGGNTEYNSALMSRLSITKDNSKSQVFLKMNSLQADDTAMYYCARDDYYFGGGAMDYWGQGTSVTVSSGGGGSGGGGSGGGGSDILLTQSPAILSVSPGERVSFACRASQTIGTNIHWYQQRTNGSPRLLIKYASESISEIPSRFSGSGSGTDFILTISSVESEDIADYYCQQSNNWPLTFGAGTKLELRGGGGSGGGGSGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIAAAYPYDVPDYAHHHHHH

SEQ ID NO: 132 - protein, 453 aa, Mus musculus
ATMETDTLLLWVLLLWVPGSTGQVQMEQSGPGLVAPSQSLSITCTVSDFSLINYGVHWVRQSPGKGLEWLGVIWTGGNTEYNSALMSRLSITKDNSKSQVFLKMNSLQADDTAMYYCARDDYYFGGGAMDYWGQGTSVTVSSGGGGSGGGGSGGGGSDILLTQSPAILSVSPGERVSFACRASQTIGTNIHWYQQRTNGSPRLLIKYASESISEIPSRFSGSGSGTDFILTISSVESEDIADYYCQQSNNWPLTFGAGTKLELRGGGGSGGGGSGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIAAAYPYDVPDYAHHHHHH

SEQ ID NO: 133 - protein, 8 aa, Mus musculus
DYKDDDDK

SEQ ID NO: 134 - DNA, 1368 bp, Mus musculus
  source 1..1368: mol_type = genomic DNA
gccaccatggaaaccgacacactgctgctgtgggtgctgcttctttgggtgcccggatctacaggccaggtgcagatggaacagtctggccctggactggtggccccatctcagagcctgagcatcacctgtaccgtgtccgacttcagcctgatcaactacggcgtgcactgggtccgacagagccctggaaaaggactggaatggctgggcgtgatctggaccggcggcaacacagagtacaacagcgccctgatgagccggctgtccatcaccaaggacaacagcaagagccaggtgttcctgaagatgaactccctgcaggccgacgacaccgccatgtactactgcgccagagatgactactacttcggcggaggcgccatggattattggggccagggaacaagcgtgaccgtgtctagcggaggcggaggatctggtggcggaggtagtggtggcggcggatctgatattctgctgacacagtcccctgccatcctgtccgtttctccaggcgagagagtgtccttcgcctgtagagcctctcagaccatcggcaccaacatccactggtatcagcagcggaccaacggcagccccagactgctgattaagtacgccagcgagagcatcagcgagatccccagcagattttctggcagcggctccggcaccgatttcatcctgacaatcagctccgtggaaagcgaggatatcgccgattactactgccagcagagcaacaactggcccctgacatttggagccggcaccaagctggaacttagaggcggcggaggttctggcggtggtggatctggcggaggtggaagcggcggaggcggctctggcggcggaggaagtgctcctattagcagccactgccggctggacaagagcaacttccagcagccttacatcaccaaccggaccttcatgctggccaaagaggccagcctggccgacaacaatactgacgtgcggctgatcggcgagaagctgtttcatggcgtgtccatgagcgagcggtgctacctgatgaagcaggtcctgaacttcaccctggaagaggtgctgttccctcagagcgaccggtttcagccctacatgcaagaggtggtgccctttctggcccggctgagcaatagactgagcacctgtcacatcgagggcgacgacctgcacatccagagaaacgtgcagaaactgaaggacaccgtgaagaagctgggcgagagcggagagatcaaggccatcggagaactggacctgctgttcatgagcctgcggaacgcctgtatcgccgctgcctatccttacgacgtgcccgattatgcccaccaccaccatcaccactgatgatga

SEQ ID NO: 135 - DNA, 855 bp, Mus musculus
  source 1..855: mol_type = genomic DNA
gccaccatggaaaccgacacactgctgctgtgggtgctgcttctttgggtgcccggatctacaggccaggtgcagatggaacagtctggccctggactggtggccccatctcagagcctgagcatcacctgtaccgtgtccgacttcagcctgatcaactacggcgtgcactgggtccgacagagccctggaaaaggactggaatggctgggcgtgatctggaccggcggcaacacagagtacaacagcgccctgatgagccggctgtccatcaccaaggacaacagcaagagccaggtgttcctgaagatgaactccctgcaggccgacgacaccgccatgtactactgcgccagagatgactactacttcggcggaggcgccatggattattggggccagggaacaagcgtgaccgtgtctagcggaggcggaggatctggtggcggaggtagtggtggcggcggatctgatattctgctgacacagtcccctgccatcctgtccgtttctccaggcgagagagtgtccttcgcctgtagagcctctcagaccatcggcaccaacatccactggtatcagcagcggaccaacggcagccccagactgctgattaagtacgccagcgagagcatcagcgagatccccagcagattttctggcagcggctccggcaccgatttcatcctgacaatcagctccgtggaaagcgaggatatcgccgattactactgccagcagagcaacaactggcccctgacatttggagccggcaccaagctggaactgagagccgccgcttatccctacgacgtgccagactatgcccaccaccaccatcaccactgatgatga

(19) United States
(12) Patent Application Publication
(10) Pub. No.: US 2026/0176582 A1
(43) Pub. Date: Jun. 25, 2026

(54) ANTIBODIES, ANTIBODY CONSTRUCTS, METHODS AND USES THEREOF

(71) Applicant: SUNNYBROOK RESEARCH INSTITUTE, Toronto (CA)

(72) Inventors: Jean GARIEPY, Toronto (CA); Amanda SPARKES, Stouffville (CA); Esther MATUS, York (CA)

(21) Appl. No.: 19/384,571

(22) Filed: Nov. 10, 2025

Related U.S. Application Data
(60) Provisional application No. 63/719,172, filed on Nov. 12, 2024.

(51) Int. Cl. C12N 5/0783; C07K 16/00; C07K 16/18; C07K 16/28

(52) U.S. Cl. CPC C12N 5/0637; C07K 16/00; C07K 16/18; C07K 16/2896; C12N 5/0636; C07K 2317/565; C07K 2317/92

(57) ABSTRACT

Antibodies are provided herein. In aspects, the antibody is capable of binding to a gastrointestinal tract (GI) tract expressed antigen. In binding to the GI tract expressed antigen, in aspects, the antibody is capable of targeting the antibody to the GI tract and in other aspects, the antibody is capable of expanding T cells. In other aspects, constructs comprising the antibody and cargo are provided. In aspects, Methods and uses are also provided.

### STATEMENT REGARDING ELECTRONIC FILING OF A SEQUENCE LISTING

A Sequence Listing in XML format, entitled SequenceListing.xml, 130,508 bytes in size, generated on Oct. 30, 2025 and filed herewith, is hereby incorporated by reference into the specification for its disclosures.

### FIELD

The present disclosure relates to antibodies or constructs thereof. In particular, the present disclosure relates to antibodies or constructs thereof that are capable of binding to gastrointestinal tract (GI) expressed antigens, and methods and uses thereof.

### BACKGROUND

The gastrointestinal (GI) tract, comprising the passage way of the digestive system that leads from the mouth to the anus, is a system in the human body to which cargo may be delivered. A common means for delivering cargo to the GI tract is via oral administration, however, oral administration has its challenges, including systemic exposure associated with undesirable or potentially harmful side effects. Targeted delivery of a cargo to the GI tract (and/or to a particular portion or section of the GI tract), however, may reduce systemic exposure and/or increase bioavailability of the cargo at the target site. Despite this, there remains an need for alternative and improved compositions and/or methods for targeted delivery to specific locations within the GI tract which overcome or mitigate at least some of the deficiencies of the prior art, or to provide a useful alternative.

The background herein is included solely to explain the context of the disclosure. This is not to be taken as an admission that any of the material referred to was published, known, or part of the common general knowledge as of the priority date.

### SUMMARY

In accordance with an aspect, there is provided an antibody capable of binding to a gastrointestinal tract (GI) tract expressed antigen.

In an aspect, the antibody comprises or consists of a polypeptide having the amino acid sequence of heavy chain complementarity-determining regions (HDCRs): HCDR1, HCDR2 and/or HCDR3 of clones 11C3, 15D6, 7A2, 2F3, 1B10, 13C5, 15A3, or 6A3, and a polypeptide having an amino acid sequence of light chain complementarity-determining regions (LCDRs): LCDR1, LCDR2 and/or LCDR3 of clones 11C3, 15D6, 7A2, 2F3, 1B10, 13C5, 15A3, or 6A3, wherein HCDR1, HCDR2, and HCDR3 are selected from:
(a) the sequence set forth in SEQ ID NO: 1, 3, and FAY of 11C3;
(b) the sequence set forth SEQ ID NO: 6, 8, and 10 of 15D6;
(c) the sequence set forth SEQ ID NO: 12, 14, and 16 of 7A2;
(d) the sequence set forth SEQ ID NO: 18, 20, and 22 of 2F3;
(e) the sequence set forth SEQ ID NO: 24, 26, and 28 of 1B10;
(f) the sequence set forth SEQ ID NO: 29, 32, and 34 of 13C5;
(g) the sequence set forth SEQ ID NO: 36, 38, and 40 of 15A3; and
(h) the sequence set forth SEQ ID NO: 42, 44, and 46 of 6A3;
and wherein LCDR1, LCDR2, and LCDR3 are selected from:
(i) the sequence set forth in SEQ ID NO: 2, 4, and 5 of 11C3;
(j) the sequence set forth SEQ ID NO: 7, 9, and 11 of 15D6;
(k) the sequence set forth SEQ ID NO: 13, 15, and 17 of 7A2;
(l) the sequence set forth SEQ ID NO: 19, 21, and 23 of 2F3;
(m) the sequence set forth SEQ ID NO: 25, 27, and 27 of 1B10;
(n) the sequence set forth SEQ ID NO: 31, 33, and 35 of 13C5;
(o) the sequence set forth SEQ ID NO: 37, 39, and 41 of 15A3; and
(p) the sequence set forth SEQ ID NO: 43, 45, and 47 of 6A3;
or functional variant that is capable of binding to the GI tract expressed antigen; or functional variant that is capable of binding to the GI tract expressed antigen.

In an aspect, the antibody comprises or consists of a polypeptide having the amino acid sequence of the heavy chain and a polypeptide having the amino acid sequence of the light chain of any one of clones 11C3, 15D6, 7A2, 2F3, 1B10, 13C5, 15A3, or 6A3:

11C3 HEAVY CHAIN
(SEQ ID NO: 48)
MLLGLKWVFFVVFYQGVHCEVKLVESGGGLVQPKGSLKLSCAASGFTFNTYAMNWIRQAPGK
GLEWVARIRSKNNYYATYYADSVKDRFTISRDDSQSMLYLQMNNLKTEDTAMYYCVSFAYWG
QGTLVTVSA
11C3 LIGHT CHAIN
(SEQ ID NO: 49)
MRFSAQLLGLLVLWIPGSTAEIVMTQAAFSNPVTLGTSASISCRSSKSLLHSNGITYLYWYLQKP
GQSPRFLIYQMSNLASGVPDRFTSSGSGTDFTLRISRVEAEDVGVYYCAQNLELPWTFGGGTK
LEIK
15D6 HEAVY CHAIN
(SEQ ID NO: 50)
MDSRLNLVFLVLILKGVQCDVQLVESGGGLVQPGGSRKLSCAASGFTFSTFGMHWVRQAPEK
GLEWVAYISSGSSSIYYADTVKGRFTISRDNPVNTLFLQMTSLRSEDTAMYYCARSGLGTSPHA
MDYWGQGTSVTVSS
15D6 LIGHT CHAIN
(SEQ ID NO: 51)
MDFQVQIFSFLLMSASVIMSRGQIVLTQSPTLMSASPGEKVTMTCSASSSVGYMYWYQQKPRS
SPKPWIYLTSILASGVPARFSGSGSGTSYSLTISSMEAEDAATYYCQQWISNPLTFGAGTKLELK
7A2 HEAVY CHAIN
(SEQ ID NO: 52)
MAVLVLFLCLAAFPSYVLSQVQMEQSGPGLVAPSQSLSITCTVSDFSLINYGVHWVRQSPGKG
LEWLGVIWTGGNTEYNSALMSRLSITKDNSKSQVFLKMNSLQADDTAMYYCARDDYYFGGGA
MDYWGQGTSVTVSS
7A2 LIGHT CHAIN
(SEQ ID NO: 53)
MVSTPQFLVFLLFWIPASRGDILLTQSPAILSVSPGERVSFACRASQTIGTNIHWYQQRTNGSPR
LLIKYASESISEIPSRFSGSGSGTDFILTISSVESEDIADYYCQQSNNWPLTFGAGTKLELR
2F3 HEAVY CHAIN
(SEQ ID NO: 54)
MEWRWIFLLLLSGTTGVHSEIQLQQSGPELVKPGASVKVSCKASGYTFTSFTLYWVKQSHGKS
LEWIGYIDPSNGGTTYNQRFKGKATLTVDKSSTTAYMHLNSLTSEDSSVYYCARGIYDGYYVG
KIFDYWGQGTTLTVSS
2F3 LIGHT CHAIN
(SEQ ID NO: 55)
METDTLLLWVLLLWVPGSTGNIVLTQSPTSLAVSLGQRATISCRASESVDNYGNSFMHWYQQK
PGQPPRLLIYLASNLESGVPARFSGSGSRTDFTLTIDPVEADDAATYYCQQNNEDPPTFGGGT
KLEIR
1B10 HEAVY CHAIN
(SEQ ID NO: 56)
MGWSYIILFLVATATDVHSQVQLQQPGAELVKPGASVKLSCKASGYTFTSYWMDWVKQRPGQ
GLEWIGEIYPSNGRTKYNEKFKNKATLTVDNSSRTAYMHLSSLTSEDSAVYYCARGGYDVYYV
GNTLDYWGQGTSVTVSS
1B10 LIGHT CHAIN
(SEQ ID NO: 57)
METDTLLLWVLLLWVPGSTGNIVLTQSPASLAVSLGQRATISCRASESIDSYGNSFMHWYHQK
PGQPPKLLIYLASNLESGVPARFSGSGSRTDFTLTIDPVEADDAATYYCLQNNGDPWTFGGGT
RLEIK
13C5 HEAVY CHAIN
(SEQ ID NO: 58)
MECNWILPFILSVTSGVYSEVQLQQSGTVLARPGASVKMSCKASGYTFTSYWMHWVKQRPG
QGLEWIGAIYPGNSDTSYNQKFKGKAKLTAVTSTSTAYMELSSLTNEDSAVYYCTIYGNYGYFD
VWGAGTTVTVSS
13C5 LIGHT CHAIN
(SEQ ID NO: 59)
MDSQAQVLMLLLLWVSGTCGDIVMSQSPSSLAVSVGEKVTMSCKSSQSLLYSSNQKNYLAWY
QQKPGQSPKLLIYWASTRESGVPDRFTGSGSGTDFTLTISSVKAEDLAVYYCQQYYSYPPYTF
GGGTKLEIK
15A3 HEAVY CHAIN
(SEQ ID NO: 60)
MEWRIFLFILSGTACVHSQDQLLQSGPELVKPGTSVKMSCRASGYTFSDDVINWVRKRTGQGL
EWIGEIYPRIGSMYYNENFKGRATLTADKSSNTVYIHLSSLTSEDSAVYFCARYLLTKGYGMDY
WGQGTSVTVSS
15A3 LIGHT CHAIN
(SEQ ID NO: 61)
METDTLLLWVLLLWVPGSTGDIVLTQSPASLAVSLGQRATISCRASKSVSPSDYSYIHWYQQKP
GQPPKLLIYLASNLESGVPARFSGSGSGTDFTLNIHPVEEEDAATYYCQHSRELPRTFGGGTKL
EIK
6A3 HEAVY CHAIN
(SEQ ID NO: 62)
MAVLGLLFCLVTFPSCVLSQVQLKQSGPGLVQPSQSLSITCTVSGFSLTSYGLHWVRQSPGKG
LEWLGFIWSGGATDYNAAFISRLSISKDNSKRQVFFKMNSLQANDTAIYFCARRDDNYALAMDY
WGQGTSVTVSS
6A3 LIGHT CHAIN
(SEQ ID NO: 63)
MDFQVQIFSFLLMSASVIMSRGQIVLTQSPALMSASPGEKVTMTCRASSSVSYIYWYQQKPRS
SPKPWIYLTSDLASGVPTRFSGSGSGTSYFLTISSMEAEDTATYYCQQWNSNPLTFGAGTKLELK

or a functional variant or fragment thereof that is capable of binding to the GI tract expressed antigen.

In an aspect, the functional variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the recited amino acid sequence.

In an aspect, the antibody comprises or consists of at 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the recited amino acid sequence.

In an aspect, the antibody is selected from IgG, IgM, IgD, IgA, or IgD.

In an aspect, the antibody is an IgG antibody having an isotype selected from IgG₁, IgG₂, IgG₃ or IgG₄.

In an aspect, the antibody is a monoclonal antibody.

In an aspect, the antibody binds to the GI tract expressed antigen.

In an aspect, the antibody has a K_{D} of between about 0.05 nM to about 20 nM for the GI tract expressed antigen.

In an aspect, the GI tract expressed antigen is Mucosal Addressin-Cell Adhesion Molecule 1 (MAdCAM-1).

In an aspect, binding to the GI tract expressed antigen (a) targets the antibody to the GI tract and/or (b) preferentially expands T cells.

In an aspect, the T cells are CD4+ and/or CD8+ T cells.

In an aspect, the T cells are CD45⁻CD27⁻ T cells.

In an aspect, the antibody binds to human MAdCAM-1.

In an aspect, the antibody is an anti-MAdCAM-1 antibody.

In an aspect, the anti-MAdCAM-1 antibody is a monoclonal anti-MAdCAM-1 antibody.

In an aspect, the anti-MAdCAM-1 antibody is a human monoclonal anti-MAdCAM-1 antibody.

In accordance with another aspect, there is provided a construct comprising an antibody and a cargo molecule, wherein the antibody is capable of binding a gastrointestinal (GI) tract expressed antigen.

In an aspect, the antibody is a functional antibody fragment that binds to the GI tract expressed antigen.

In an aspect, the functional antibody fragment is fused to the cargo molecule.

In an aspect, the cargo molecule does not impact or impair the ability of the functional antibody fragment to bind to the GI tract expressed antigen.

In an aspect, the functional antibody fragment has a Kp of between about 0.05 nM to about 20 nM for the GI tract expressed antigen.

In an aspect, the functional antibody fragment is selected from a scFv, a Fab′, a Fab, a F(ab′) 2, a Fv, or a scFab.

In an aspect, the functional antibody fragment is a scFv.

In an aspect, the scFv comprises or consists of a polypeptide having the sequence:

(SEQ ID NO: 128)
QVQMEQSGPGLVAPSQSLSITCTVSDFSLINYGVHWVRQSPGKGLEWLG
VIWTGGNTEYNSALMSRLSITKDNSKSQVFLKMNSLQADDTAMYYCARD
DYYFGGGAMDYWGQGTSVTVSSGGGGSGGGGSGGGGSDILLTQSPAILS
VSPGERVSFACRASQTIGTNIHWYQQRTNGSPRLLIKYASESISEIPSR
FSGSGSGTDFILTISSVESEDIADYYCQQSNNWPLTFGAGTKLELRAAA
YPYDVPDYAHHHHHH

In an aspect, the construct further comprises a secretion sequence, as set forth in SEQ ID NO: 130, at the N-terminus of sequence.

In an aspect, binding to the GI tract expressed antigen (a) targets the construct to the GI tract and/or (b) preferentially expands T cells.

In an aspect, the T cells are CD4+ and/or CD8+ T cells.

In an aspect, the T cells are CD45⁻CD27⁻ T cells.

In an aspect, the GI tract expressed antigen is Mucosal Addressin-Cell Adhesion Molecule 1 (MAdCAM-1).

In an aspect, the scFv is capable of binding or binds to MAdCAM1.

In an aspect, the cargo molecule is selected from proteins, peptides, nucleic acids, amino acids, nucleosides, antibodies, antibody fragments, antibody ligands, peptide nucleic acids, small organic molecules, lipids, hormones, drugs, enzymes, lectin, cell adhesion molecule, antibody epitope, enzyme substrates, enzyme inhibitors, coenzymes, inorganic molecules, carbohydrates, such as polysaccharides and monosaccharides, or a combination thereof.

In an aspect, the cargo molecule is a protein.

In an aspect, the cargo molecule is IL-22, IL-18 bp or an IL-23 blocker such as Ustekinumab, Risankizumab.

In an aspect, when the construct is targeted to the GI tract expressed antigen, the construct is capable of limiting off-target effects of the cargo molecule.

In an aspect, when the construct is targeted to the GI tract expressed antigen, the construct can allow for accumulation of the cargo molecule at a targeted site in the GI tract.

In an aspect, the targeted site in the GI tract comprises the small intestine, the large intestine and/or the colon.

In an aspect, at least one of the following is true:
(a) the construct allows for inhibition of IL-18; (b) the construct allows for production of IL-10; (c) the construct induces STAT3 phosphorylation; (d) the construct reduces IFNγ secretion.

In an aspect, the construct has the sequence:

(SEQ ID NO: 132)
ATMETDTLLLWVLLLWVPGSTGQVQMEQSGPGLVAPSQSLSITCTVSDF
SLINYGVHWVRQSPGKGLEWLGVIWTGGNTEYNSALMSRLSITKDNSKS
QVFLKMNSLQADDTAMYYCARDDYYFGGGAMDYWGQGTSVTVSSGGGGS
GGGGSGGGGSDILLTQSPAILSVSPGERVSFACRASQTIGTNIHWYQQR
TNGSPRLLIKYASESISEIPSRFSGSGSGTDFILTISSVESEDIADYYC
QQSNNWPLTFGAGTKLELRGGGGSGGGGSGGGGSGGGGSGGGGSAPISS
HCRLDKSNFQQPYITNRTFMLAKEASLADNNTDVRLIGEKLFHGVSMSE
RCYLMKQVLNFTLEEVLFPQSDRFQPYMQEVVPFLARLSNRLSTCHIEG
DDLHIQRNVQKLKDTVKKLGESGEIKAIGELDLLFMSLRNACIAAAYPY
DVPDYAHHHHHH

In another aspect, there is a polynucleotide encoding the construct described herein.

In an aspect, the polynucleotide has the sequence:

(SEQ ID NO: 134)
GCCACCATGGAAACCGACACACTGCTGCTGTGGGTGCTGCTTCTTTGGG
TGCCCGGATCTACAGGCCAGGTGCAGATGGAACAGTCTGGCCCTGGACT
GGTGGCCCCATCTCAGAGCCTGAGCATCACCTGTACCGTGTCCGACTTC
AGCCTGATCAACTACGGCGTGCACTGGGTCCGACAGAGCCCTGGAAAAG
GACTGGAATGGCTGGGCGTGATCTGGACCGGCGGCAACACAGAGTACAA
CAGCGCCCTGATGAGCCGGCTGTCCATCACCAAGGACAACAGCAAGAGC
CAGGTGTTCCTGAAGATGAACTCCCTGCAGGCCGACGACACCGCCATGT
ACTACTGCGCCAGAGATGACTACTACTTCGGCGGAGGCGCCATGGATTA
TTGGGGCCAGGGAACAAGCGTGACCGTGTCTAGCGGAGGCGGAGGATCT
CTGGTGGCGGAGGTAGTGGTGGCGGCGGATCTGATATTGCTGACACAGT
CCCCTGCCATCCTGTCCGTTTCTCCAGGCGAGAGAGTGTCCTTCGCCTG
TAGAGCCTCTCAGACCATCGGCACCAACATCCACTGGTATCAGCAGCGG
ACCAACGGCAGCCCCAGACTGCTGATTAAGTACGCCAGCGAGAGCATCA
GCGAGATCCCCAGCAGATTTTCTGGCAGCGGCTCCGGCACCGATTTCAT
CCTGACAATCAGCTCCGTGGAAAGCGAGGATATCGCCGATTACTACTGC
CAGCAGAGCAACAACTGGCCCCTGACATTTGGAGCCGGCACCAAGCTGG
AACTTAGAGGCGGCGGAGGTTCTGGCGGTGGTGGATCTGGCGGAGGTGG
AAGCGGCGGAGGCGGCTCTGGCGGCGGAGGAAGTGCTCCTATTAGCAGC
CACTGCCGGCTGGACAAGAGCAACTTCCAGCAGCCTTACATCACCAACC
GGACCTTCATGCTGGCCAAAGAGGCCAGCCTGGCCGACAACAATACTGA
CGTGCGGCTGATCGGCGAGAAGCTGTTTCATGGCGTGTCCATGAGCGAG
CGGTGCTACCTGATGAAGCAGGTCCTGAACTTCACCCTGGAAGAGGTGC
TGTTCCCTCAGAGCGACCGGTTTCAGCCCTACATGCAAGAGGTGGTGCC
CTTTCTGGCCCGGCTGAGCAATAGACTGAGCACCTGTCACATCGAGGGC
GACGACCTGCACATCCAGAGAAACGTGCAGAAACTGAAGGACACCGTGA
AGAAGCTGGGCGAGAGCGGAGAGATCAAGGCCATCGGAGAACTGGACCT
GCTGTTCATGAGCCTGCGGAACGCCTGTATCGCCGCTGCCTATCCTTAC
GACGTGCCCGATTATGCCCACCACCACCATCACCACTGATGATGA

In accordance with another aspect, there is provided a vector comprising the polynucleotide described herein.

In accordance with another aspect, there is a host cell comprising the vector described herein.

In accordance with another aspect, there is a composition comprising the construct described herein and a carrier.

In accordance with another aspect, there is provided a use of the antibody described herein or the construct described herein for binding to a GI tract expressed antigen.

In an aspect, the GI tract expressed antigen is Mucosal Addressin-Cell Adhesion Molecule 1 (MAdCAM-1).

In an aspect, the binding allows for (a) targeting the construct to the GI tract and/or (b) preferentially expanding T cells.

In an aspect, the T cells are CD4+ and/or CD8+ T cells.

In an aspect, the T cells are CD45⁻CD27⁻ T cells.

In an aspect, the use allows for accumulation of the cargo molecule in a targeted site in the GI tract.

In an aspect, the targeted site in the GI tract comprises the small intestine, the large intestine and/or the colon.

In accordance with another aspect, there is provided a method of targeting the antibody described herein or the construct described herein to a GI tract expressed antigen, the method comprising administering the antibody or the construct to a subject and targeting the antibody or the construct to the GI tract of the subject.

In an aspect, the method further comprises delivering the antibody or the construct to the GI tract.

In an aspect, the GI tract expressed antigen is Mucosal Addressin-Cell Adhesion Molecule 1 (MAdCAM-1).

In an aspect, the method further comprises allowing accumulation of the cargo molecule in a targeted site in the GI tract.

In an aspect, the targeted site in the GI tract comprises the small intestine, the large intestine and/or the colon.

In accordance with another aspect, there is provided a method of expanding T cells, the method comprising binding the antibody described herein or the construct described herein to a gastrointestinal (GI) tract expressed antigen, thereby expanding the T cells.

In an aspect, the T cells are CD4+ and/or CD8+ T cells.

In an aspect, the T cells are CD45⁻CD27⁻ T cells.

In accordance with another aspect, there is provided a use of the antibody described herein or the construct described herein for expanding T cells, wherein the antibody or the construct is for binding to a gastrointestinal (GI) tract expressed antigen, thereby expanding the T cells.

In an aspect, the T cells are CD4+ and/or CD8+ T cells.

In an aspect, the T cells are CD45⁻CD27⁻ T cells.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 Sequence alignment and production of anti-hMAdCAM-1 mAbs. (A) Sequence alignments of the variable regions of heavy chains of mouse anti-human MAdCAM-1 clustered, using Clustal Omega software, based on sequence similarity. (B) SDS-PAGE gel and Western blot of anti-hMAdCAM-1 mAbs under reducing conditions. Bands on Western blots were detected using a HRP-conjugated antibody that recognizes heavy and light chains of murine IgGs.

FIG. 2 Binding Characterization of mouse anti-human MAdCAM-1 mAbs. (A) SPR single-cycle kinetics sensorgrams (in red) and fitted curves (in black) with equilibrium (KD), association (kon) and dissociation (koff) rate constants depicting the binding of the mAbs to human MAdCAM-1. (B) Detection of hMAdCAM-1 (non-reducing conditions) using the mAbs as detecting reagents. 17F5 is a commercially available mouse anti human MAdCAM-1 mAb and was used as a positive control. (C) Binding of anti hMAdCAM-1 mAbs to Expi293F cells transiently expressing hMAdCAM-1; Plots depict the binding of a commercial anti-hMAdCAM-1 antibody (17F5), and all generated mAbs to Expi293Fcells transiently transfected to express hMAdCAM on the cell surface (red) in comparison to binding to non-transfected cells (blue). (D). Binding of anti-hMAdCAM-1 mAbs to human small intestine. Clones 1B10, 11C3, 2F3 and 7A2 detected hMAdCAM-1 expression (arrows) in human small intestine tissue evidenced by histological staining. All other clones were negative (data not shown).

FIG. 3 Anti-hMAdCAM-1 mAbs 1B10, 11C3, 2F3 and 7A2 inhibit MAdCAM-1 T cell co-stimulation. CFSE labeled T cells isolated from PBMCs isolated from healthy donors were stimulated with a suboptimal concentration of plate-bound anti-CD3, anti-CD3 co-coated with anti-CD28 or anti-CD3 co-coated with hMAdCAM-1.Fc in the presence of retinoic acid and with or without the addition of mAbs or a negative IgG control. After 4 days in culture (A) cell proliferation in the CD4+ population was measured as a function of diluting CFSE. mAbs 1B10, 11C3, 2F3 and 7A2 significantly reduced MAdCAM-1 mediated proliferation of the total CD4+ T cell population when compared to MAdCAM-1.Fc in the absence of mAbs. (B) Phenotypic analysis of the CD4+ T cell population indicates the aforementioned 4 clones significantly reduce the expansion of MAdCAM-1 induced central memory (CD45RA⁻, CD27+) cells in favor of a more differentiated phenotype. n=4, 2-way anova with multiple comparison, * p<0.05, ** p<0.005, **** p<0.0005.

FIG. 4 Design and purification of 7A2scFv-IL22. (A) Diagram depicting the structure of hMAdCAM-1 scFv 7A2scFv-IL22. This bispecific protein is a linear arrangement of an N-terminal human MAdCAM-1-targeting scFv connected by a short flexible linker to murine IL22. The scFv domain is depicted as a linear assembly of variable heavy (VH) and variable light (VL) chains. A histidine tag (6× His) and a hemagglutinin (HA) tag were inserted at the C-terminus to facilitate purification and detection. (B) Predicted Ribbon Structure of hMAdCAM-1 scFv7A2scFv-IL22 using I-TASSER software. The scFv domain is depicted in red, the linker in blue, IL22 in green, the AAA linker in pink, the HA tag in orange and the HIS tag in purple. (C) hMAdCAM-1 scFv 7A2scFv-IL22 was produced in Expi293F cells and purified by Ni-NTA affinity chromatography. Purification product is shown using SDS-PAGE (left panel; Coomassie staining) and by Western blot (right panel; detected using an anti-His tag antibody). The protein migrates as a ˜57 kDa band (D) A bridging ELISA showing the dose-dependent increase in hMAdCAM-1 scFv 7A2scFv-IL22 simultaneously binding to both its targets. Indicated doses of hMAdCAM-1 scFv 7A2scFv-IL22 were dispensed into wells of a 96-well plate pre-coated with human IL22Rα. Biotinylated MAdCAM-1 and streptavidin-HRP were subsequently added to detect the formation of trimolecular complexes (n=3). (E) Staining of healthy human small intestine sections using biotinylated hMAdCAM-1 scFv 7A2scFv-IL22 followed by streptavidin-HRP and detected by DAB staining (left) or staining with streptavidin-HRP and DAB in the absence of biotinylated hMAdCAM-1 scFv 7A2scFv-IL22 (right). (F) The function of the IL22 domain of 7A2scFv-IL22 was confirmed through its ability to increase IL-10 in vitro relative to the parent antibody 7A2 (n=3. Welch's T Test. *** p<0.001

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “antibody”, also referred to in the art as “immunoglobulin” (Ig), used herein refers to a protein constructed from paired heavy and light polypeptide chains; various Ig isotypes exist, including IgA, IgD, IgE, IgG, such as IgG₁, IgG₂, IgG₃, and IgG₄, and IgM. It will be understood that the antibody may be from any species, including human, mouse, rat, monkey, llama, or shark. When an antibody is correctly folded, each chain folds into a number of distinct globular domains joined by more linear polypeptide sequences. For example, in the case of IgGs, the immunoglobulin light chain folds into a variable (VL) and a constant (CL) domain, while the heavy chain folds into a variable (VA) and three constant (CH1, CH2, CH3) domains. Interaction of the heavy and light chain variable domains (VH and VL) results in the formation of an antigen binding region (Fv). Each domain has a well-established structure familiar to those of skill in the art.

The term “monoclonal antibody” as used herein refers to antibodies that are substantially identical to amino acid sequence or are derived from the same genetic source. A monoclonal antibody composition displays a binding specificity and affinity for a particular epitope, or binding specificities and affinities for specific epitopes.

The term “chimeric antibody” is an antibody molecule in which (a) the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen, while having reduced antigenicity in human as compared to the original mouse antibody.

A “humanized” antibody, as used herein, is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). Additional framework region modifications may be made within the human framework sequences as well as within the CDR sequences derived from the germline of another mammalian species. The humanized antibodies of the invention may include amino acid residues not encoded by human sequences (e.g., mutations introduced by random or site-specific mutagenesis in vitro or by somatic mutation in vivo, or a conservative substitution to promote stability or manufacturing). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun., 28:489-498, 1991; and Padlan, Molec. Immun., 31:169-217, 1994. Other examples of antibody engineering technology include, but are not limited to Xoma technology disclosed in U.S. Pat. No. 5,766,886.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein refers to at least one portion of an intact antibody, or recombinant variants thereof, which may include any suitable antigen-binding antibody fragment known in the art. The antibody fragment may be a naturally-occurring antibody fragment, or may be obtained by manipulation of a naturally-occurring antibody or by using recombinant methods. The term “functional fragment” or “functional antibody fragment” or “antigen-binding fragment” refers to an antibody fragment comprising at least an antigen-binding domain, e.g., that part of the variable region of an intact antibody, that is sufficient to confer recognition and specific binding of the functional antibody fragment to a target, such as the antigenic determinant of an antigen. An “antigen-binding region” or “antigen-binding domain” of an antibody typically is found in one or more hypervariable region(s) of an antibody, i.e., the CDR1, CDR2, and/or CDR3 regions; however, the variable “framework” regions can also play an important role in antigen binding, such as by providing a scaffold for the CDRs. Examples of functional antibody fragments include, but are not limited to, Fab′, F(ab′) 2, and Fv fragments, single-chain Fv (scFv) antibody fragments, Fc, single-chain Fc, Fab, single-chain Fab, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide bridge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Complementarity Determining Regions” (“CDRs”) are amino acid sequences with boundaries determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 24-34 (LCDR1), 50-56 (LCDR2), and 89-97 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to “Kabat”). Under IMGT, the CDRs of an antibody can be determined using the program IMGT/DomainGap Align.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Variants” are biologically active constructs, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

The constructs described herein may include modifications. Such modifications include, but are not limited to, conjugation to an effector molecule. Modifications further include, but are not limited to conjugation to detectable reporter moieties. Modifications that extend half-life (e.g., pegylation) are also included. Proteins and non-protein agents may be conjugated to the constructs by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR) 25, 508-514 (1991), both of which are incorporated by reference herein.

“Active” or “activity” for the purposes herein refers to a biological and/or an immunological activity of the constructs described herein, wherein “biological” activity refers to a biological function (either inhibitory or stimulatory) caused by the constructs.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further agents includes simultaneous (concurrent) and consecutive administration in any order. The term “administration” (e.g., “administering” a compound) in reference to an antibody, construct, composition and/or formulation disclosed herein includes, for example, introducing the antibody, construct, composition and/or formulation into the system of the mammal. In specific aspects, the introduction is related to the delivery of the antibody, construct or composition to the GI tract of the mammal, and in further specific aspects, it is delivered to a specific target site in the GI tract.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration. The term “pharmaceutically acceptable” includes those compounds, materials, compositions, and/or dosage forms which are, within the scope of sound medical judgment, suitable for use in contact with the tissues of humans and animals without excessive toxicity, irritation, allergic response, or other problem or complication commensurate with a reasonable benefit/risk ratio.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

When introducing elements disclosed herein, the articles “a”, “an”, “the”, and “said” are intended to mean that there may be one or more of the elements.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. It will be understood that any embodiments described as “comprising” certain components may also “consist of” or “consist essentially of,” these components, wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known acceptable additive, excipient, diluent, carrier, and the like, suitable for the composition described herein. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation, such as any specific compounds or method steps, whether implicitly or explicitly defined herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

The phrase “at least one of” is understood to be one or more. The phrase “at least one of . . . and . . . ” is understood to mean at least one of the elements listed or a combination thereof, if not explicitly listed. For example, “at least one of A, B, and C” is understood to mean A alone or B alone or C alone or a combination of A and B or a combination of A and C or a combination of B and C or a combination of A, B, and C.

Described herein are antibodies that are capable of binding to a gastrointestinal (GI) tract expressed antigen. Said antigen may be an in vitro expressed antigen or it may be an in vivo expressed antigen. In aspects, the antibodies may be used for targeting to GI tract expressed antigens. In this way, the antibody can be targeted to a target site in the GI tract as described in further detail below. In other aspects, the antibodies may be used for the preferential expansion of immunological cells, such as T cells. In this way, the antibody can be used as a blocking antibody such that, when the antibody binds to the GI tract expressed antigen, the antibody favours the expansion of particular subsets of T cells, as described in further detail below.

A number of exemplary antibodies have been prepared and tested as described and exemplified herein and other antibodies with similar binding and effector functions are also encompassed herein. As exemplified herein, the antibodies 11C3, 15D6, 7A2, 2F3, 1B10, 13C5, 15A3, and 6A3 have been produced and are capable of binding to GI tract expressed antigens and/or preferentially expanding T cells.

While the GI tract includes the stomach, the duodenum, the jejunum, the ileum, the cecum, the ascending colon, and the traverse colon, the GI tract expressed antigen described herein is typically expressed in the small or large intestines, and may also be referred to as an intestinal antigen. The GI tract expressed antigens may be expressed, for example by, high endothelial venules (HEVs) of Peyer's patches, endothelial cells in postcapillary venules of both the small and large intestinal lamina propria, and mesenteric lymph nodes in the GI tract. Thus, the antibody described herein may be targeted anywhere along the GI tract depending on the expression pattern of the GI tract expressed antigen that the antibody is generated against.

In aspects, the antibody comprises or consists of a polypeptide having an amino acid sequence of heavy chain complementarity-determining regions (HDCRs): HCDR1, HCDR2 and/or HCDR3 of clones 11C3, 15D6, 7A2, 2F3, 1B10, 13C5, 15A3, or 6A3, and a polypeptide having an amino acid sequence of light chain complementarity-determining regions (LCDRs): LCDR1, LCDR2 and/or LCDR3 of clones 11C3, 15D6, 7A2, 2F3, 1B10, 13C5, 15A3, or 6A3, wherein HCDR1, HCDR2, and HCDR3 are selected from:
(a) the sequence set forth in SEQ ID NO: 1, 3, and FAY of 11C3;
(b) the sequence set forth SEQ ID NO: 6, 8, and 10 of 15D6;
(c) the sequence set forth SEQ ID NO: 12, 14, and 16 of 7A2;
(d) the sequence set forth SEQ ID NO: 18, 20, and 22 of 2F3;
(e) the sequence set forth SEQ ID NO: 24, 26, and 28 of 1B10;
(f) the sequence set forth SEQ ID NO: 29, 32, and 34 of 13C5;
(g) the sequence set forth SEQ ID NO: 36, 38, and 40 of 15A3; and
(h) the sequence set forth SEQ ID NO: 42, 44, and 46 of 6A3;
and wherein LCDR1, LCDR2, and LCDR3 are selected from:
(i) the sequence set forth in SEQ ID NO: 2, 4, and 5 of 11C3;
(j) the sequence set forth SEQ ID NO: 7, 9, and 11 of 15D6;
(k) the sequence set forth SEQ ID NO: 13, 15, and 17 of 7A2;
(l) the sequence set forth SEQ ID NO: 19, 21, and 23 of 2F3;
(m) the sequence set forth SEQ ID NO: 25, 27, and 27 of 1B10;
(n) the sequence set forth SEQ ID NO: 31, 33, and 35 of 13C5;
(o) the sequence set forth SEQ ID NO: 37, 39, and 41 of 15A3; and
(p) the sequence set forth SEQ ID NO: 43, 45, and 47 of 6A3.

Heavy and Light Chain CDR1-3 sequences:

11C3 HEAVY CHAIN CDR 1
(SEQ ID NO: 1)
TYAMN
11C3 LIGHT CHAIN CDR 1
(SEQ ID NO: 2)
RSSKSLLHSNGITYLY
11C3 HEAVY CHAIN CDR 2
(SEQ ID NO: 3)
RIRSKNNYYATYYADSVKD
11C3 LIGHT CHAIN CDR 2
(SEQ ID NO: 4)
QMSNLAS
11C3 HEAVY CHAIN CDR 3:
FAY
11C3 LIGHT CHAIN CDR 3
(SEQ ID NO: 5)
AQNLELPWT
15D6 HEAVY CHAIN CDR 1
(SEQ ID NO: 6)
TFGMH
15D6 LIGHT CHAIN CDR 1
(SEQ ID NO: 7)
SASSSVGYMY
15D6 HEAVY CHAIN CDR 2
(SEQ ID NO: 8)
YISSGSSSIYYADTVKG
15D6 LIGHT CHAIN CDR 2
(SEQ ID NO: 9)
LTSILAS
15D6 HEAVY CHAIN CDR 3
(SEQ ID NO: 10)
SGLGTSPHAMDY
15D6 LIGHT CHAIN CDR 3
(SEQ ID NO: 11)
QQWISNPLT
7A2 HEAVY CHAIN CDR 1
(SEQ ID NO: 12)
NYGVH
7A2 LIGHT CHAIN CDR 1
(SEQ ID NO: 13)
RASQTIGTNIH
7A2 HEAVY CHAIN CDR 2
(SEQ ID NO: 14)
VIWTGGNTEYNSALMS
7A2 LIGHT CHAIN CDR 2
(SEQ ID NO: 15)
YASESIS
7A2 HEAVY CHAIN CDR 3
(SEQ ID NO: 16)
DDYYFGGGAMDY
7A2 LIGHT CHAIN CDR 3
(SEQ ID NO: 17)
QQSNNWPLT
2F3 HEAVY CHAIN CDR 1
(SEQ ID NO: 18)
SFTLY
2F3 LIGHT CHAIN CDR 1
(SEQ ID NO: 19)
RASESVDNYGNSFMH
2F3 HEAVY CHAIN CDR 2
(SEQ ID NO: 20)
YIDPSNGGTTYNQRFKG
2F3 LIGHT CHAIN CDR 2
(SEQ ID NO: 21)
LASNLES
2F3 HEAVY CHAIN CDR 3
(SEQ ID NO: 22)
GIYDGYYVGKIFDY
2F3 LIGHT CHAIN CDR 3
(SEQ ID NO: 23)
QQNNEDPPT
1B10 HEAVY CHAIN CDR 1
(SEQ ID NO: 24)
SYWMD
1B10 LIGHT CHAIN CDR 1
(SEQ ID NO: 25)
RASESIDSYGNSFMH
1B10 HEAVY CHAIN CDR 2
(SEQ ID NO: 26)
EIYPSNGRTKYNEKFKN
1B10 LIGHT CHAIN CDR 2
(SEQ ID NO: 27)
LASNLES
1B10 HEAVY CHAIN CDR 3
(SEQ ID NO: 28)
GGYDVYYVGNTLDY
1B10 LIGHT CHAIN CDR 3
(SEQ ID NO: 29)
LQNNGDPWT
13C5 HEAVY CHAIN CDR 1
(SEQ ID NO: 30)
SYWMH
13C5 LIGHT CHAIN CDR 1
(SEQ ID NO: 31)
KSSQSLLYSSNQKNYLA
13C5 HEAVY CHAIN CDR 2
(SEQ ID NO: 32)
AIYPGNSDTSYNQKFKG
13C5 LIGHT CHAIN CDR 2
(SEQ ID NO: 33)
WASTRES
13C5 HEAVY CHAIN CDR 3
(SEQ ID NO: 34)
YGNYGYFDV
13C5 LIGHT CHAIN CDR 3
(SEQ ID NO: 35)
QQYYSYPPYT
15A3 HEAVY CHAIN CDR 1
(SEQ ID NO: 36)
DDVIN
15A3 LIGHT CHAIN CDR 1
(SEQ ID NO: 37)
RASKSVSPSDYSYIH
15A3 HEAVY CHAIN CDR 2
(SEQ ID NO: 38)
EIYPRIGSMYYNENFKG
15A3 LIGHT CHAIN CDR 2
(SEQ ID NO: 39)
LASNLES
15A3 HEAVY CHAIN CDR 3
(SEQ ID NO: 40)
YLLTKGYGMDY
15A3 LIGHT CHAIN CDR 3
(SEQ ID NO: 41)
QHSRELPRT
6A3 HEAVY CHAIN CDR 1
(SEQ ID NO: 42)
SYGLH
6A3 LIGHT CHAIN CDR 1
(SEQ ID NO: 43)
RASSSVSYIY
6A3 HEAVY CHAIN CDR 2
(SEQ ID NO: 44)
FIWSGGATDYNAAFIS
6A3 LIGHT CHAIN CDR 2
(SEQ ID NO: 45)
LTSDLAS
6A3 HEAVY CHAIN CDR 3
(SEQ ID NO: 46)
RDDNYALAMDY
6A3 LIGHT CHAIN CDR 3
(SEQ ID NO: 47)
QQWNSNPLT

Functional variants or fragments of these sequences that specifically bind to the GI tract expressed antigens and/or preferentially expand T cells, are also contemplated for use herein.

In addition, other antibodies comprising one or more of the CDRs of these antibodies are contemplated.

In another aspect, the antibody comprises or consists of a polypeptide having the amino acid sequence of the heavy chain and a polypeptide having the amino acid sequence of the light chain of any one of clones 11C3, 15D6, 7A2, 2F3, 1B10, 13C5, 15A3, or 6A3:

Heavy and Light chain polypeptide sequences of the clones:

11C3 HEAVY CHAIN
(SEQ ID NO: 48)
MLLGLKWVFFVVFYQGVHCEVKLVESGGGLVQPKGSLKLSCAASGFTF
NTYAMNWIRQAPGKGLEWVARIRSKNNYYATYYADSVKDRFTISRDDS
QSMLYLQMNNLKTEDTAMYYCVSFAYWGQGTLVTVSA
11C3 LIGHT CHAIN
(SEQ ID NO: 49)
MRFSAQLLGLLVLWIPGSTAEIVMTQAAFSNPVTLGTSASISCRSSKS
LLHSNGITYLYWYLQKPGQSPRFLIYQMSNLASGVPDRFTSSGSGTDF
TLRISRVEAEDVGVYYCAQNLELPWTFGGGTKLEIK
15D6 HEAVY CHAIN
(SEQ ID NO: 50)
MDSRLNLVFLVLILKGVQCDVQLVESGGGLVQPGGSRKLSCAASGFTF
STFGMHWVRQAPEKGLEWVAYISSGSSSIYYADTVKGRFTISRDNPVN
TLFLQMTSLRSEDTAMYYCARSGLGTSPHAMDYWGQGTSVTVSS
15D6 LIGHT CHAIN
(SEQ ID NO: 51)
MDFQVQIFSFLLMSASVIMSRGQIVLTQSPTLMSASPGEKVTMTCSAS
SSVGYMYWYQQKPRSSPKPWIYLTSILASGVPARFSGSGSGTSYSLTI
SSMEAEDAATYYCQQWISNPLTFGAGTKLELK
7A2 HEAVY CHAIN
(SEQ ID NO: 52)
MAVLVLFLCLAAFPSYVLSQVQMEQSGPGLVAPSQSLSITCTVSDFSL
INYGVHWVRQSPGKGLEWLGVIWTGGNTEYNSALMSRLSITKDNSKSQ
VFLKMNSLQADDTAMYYCARDDYYFGGGAMDYWGQGTSVTVSS
7A2 LIGHT CHAIN
(SEQ ID NO: 53)
MVSTPQFLVFLLFWIPASRGDILLTQSPAILSVSPGERVSFACRASQTI
GTNIHWYQQRTNGSPRLLIKYASESISEIPSRFSGSGSGTDFILTISSV
ESEDIADYYCQQSNNWPLTFGAGTKLELR
2F3 HEAVY CHAIN
(SEQ ID NO: 54)
MEWRWIFLLLLSGTTGVHSEIQLQQSGPELVKPGASVKVSCKASGYTFT
SFTLYWVKQSHGKSLEWIGYIDPSNGGTTYNQRFKGKATLTVDKSSTTA
YMHLNSLTSEDSSVYYCARGIYDGYYVGKIFDYWGQGTTLTVSS
2F3 LIGHT CHAIN
(SEQ ID NO: 55)
METDTLLLWVLLLWVPGSTGNIVLTQSPTSLAVSLGQRATISCRASESV
DNYGNSFMHWYQQKPGQPPRLLIYLASNLESGVPARFSGSGSRTDFTLT
IDPVEADDAATYYCQQNNEDPPTFGGGTKLEIR
1B10 HEAVY CHAIN
(SEQ ID NO: 56)
MGWSYIILFLVATATDVHSQVQLQQPGAELVKPGASVKLSCKASGYTFT
SYWMDWVKQRPGQGLEWIGEIYPSNGRTKYNEKFKNKATLTVDNSSRTA
YMHLSSLTSEDSAVYYCARGGYDVYYVGNTLDYWGQGTSVTVSS
1B10 LIGHT CHAIN
(SEQ ID NO: 57)
METDTLLLWVLLLWVPGSTGNIVLTQSPASLAVSLGQRATISCRASESI
DSYGNSFMHWYHQKPGQPPKLLIYLASNLESGVPARFSGSGSRTDFTLT
IDPVEADDAATYYCLQNNGDPWTFGGGTRLEIK
13C5 HEAVY CHAIN
(SEQ ID NO: 58)
MECNWILPFILSVTSGVYSEVQLQQSGTVLARPGASVKMSCKASGYTFT
SYWMHWVKQRPGQGLEWIGAIYPGNSDTSYNQKFKGKAKLTAVTSTSTA
YMELSSLTNEDSAVYYCTIYGNYGYFDVWGAGTTVTVSS
13C5 LIGHT CHAIN
(SEQ ID NO: 59)
MDSQAQVLMLLLLWVSGTCGDIVMSQSPSSLAVSVGEKVTMSCKSSQSL
LYSSNQKNYLAWYQQKPGQSPKLLIYWASTRESGVPDRFTGSGSGTDFT
LTISSVKAEDLAVYYCQQYYSYPPYTFGGGTKLEIK
15A3 HEAVY CHAIN
(SEQ ID NO: 60)
MEWRIFLFILSGTACVHSQDQLLQSGPELVKPGTSVKMSCRASGYTFSD
DVINWVRKRTGQGLEWIGEIYPRIGSMYYNENFKGRATLTADKSSNTVY
IHLSSLTSEDSAVYFCARYLLTKGYGMDYWGQGTSVTVSS
15A3 LIGHT CHAIN
(SEQ ID NO: 61)
METDTLLLWVLLLWVPGSTGDIVLTQSPASLAVSLGQRATISCRASKSV
SPSDYSYIHWYQQKPGQPPKLLIYLASNLESGVPARFSGSGSGTDFTLN
IHPVEEEDAATYYCQHSRELPRTFGGGTKLEIK
6A3 HEAVY CHAIN
(SEQ ID NO: 62)
MAVLGLLFCLVTFPSCVLSQVQLKQSGPGLVQPSQSLSITCTVSGFSLT
SYGLHWVRQSPGKGLEWLGFIWSGGATDYNAAFISRLSISKDNSKRQVF
FKMNSLQANDTAIYFCARRDDNYALAMDYWGQGTSVTVSS
6A3 LIGHT CHAIN
(SEQ ID NO: 63)
MDFQVQIFSFLLMSASVIMSRGQIVLTQSPALMSASPGEKVTMTCRASS
SVSYIYWYQQKPRSSPKPWIYLTSDLASGVPTRFSGSGSGTSYFLTISS
MEAEDTATYYCQQWNSNPLTFGAGTKLELK

Functional variants or fragments of these sequences that specifically bind to the GI tract expressed antigen and/or preferentially expand T cells, are also contemplated for use herein.

The functional variants described herein may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the recited amino acid sequence. In a similar way, the antibody described herein may comprise or consists of at 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the recited amino acid sequence.

The antibody can be of any class of antibody, such as those selected from IgG, IgM, IgD, IgA, or IgD. In typical aspects, the antibody is of the class IgG. In addition, the antibody described herein may be any isotype of IgG, such as for example, the antibody may be a IgG antibody and the IgG antibody is IgG₁, IgG₂, IgGs or IgG₄. In typical aspects, however, the antibody described herein is IgG₁.

The term “antibody”, as used herein, includes for example, monoclonal antibodies, humanized antibodies, or chimeric antibodies. In typical aspects, the antibody is a monoclonal antibody. In aspects, the monoclonal antibodies include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984), incorporated herein by reference). The antibody may of any mammalian source, such as, humans, other higher primates, domestic and farm animals, and zoo, sports, or pet animals, such as mice, dogs, cats, cattle, horses, sheep, pigs, goats, or rabbits. The mammalian source for the antibody is typically human.

The antibody described herein typically has affinity for, or binds to, the GI tract expressed antigen described herein. By “affinity”, it is meant to refer to the strength of the sum of total noncovalent interactions between a single binding site or a molecule, e.g., an antibody or a functional antibody fragment, and its binding partner, e.g., an antigen. The affinity can generally be represented by the dissociation constant (K_{D}). Affinity can be measured by common methods known in the art, including those described herein.

In aspects, the antibody has a Kp of between about 0.05 nM to about 20 nM for the GI tract expressed antigen, such as a K_{D} of about 0.05 nm, about 0.1 nM, about 0.15 nM, about 0.20 nm, about 0.25 nM, about 0.30 nM, about 0.35 nM, about 0.40 nM, about 0.45 nM, about 0.5 nM, about 0.55 nM, about 0.60 nM, about 0.65 nM, about 0.7 nM, about 0.75 nM, about 0.80 nM, about 0.85 nM, about 0.90 nM, about 0.95 nM, about 1 nM, about 1.5 nM, about 2.0 nM, about 2.5 nM, about 3.0 nM, about 3.5 nM, about 4.0 nM, about 4.5 nM, about 5.0 nM, about 5.5 nM, about 6.0 nM, about 6.5 nM, about 7.0 nM, about 7.5 nM, about 8.0 nM, about 8.5 nM, about 9.0 nM, about 9.5 nM, about 10 nM, about 10.5 nM, about 11.0 nM, about 11.5 nM, about 12.0 nM, about 12.5 nM, about 13.0 nM, about 13.5 nM, about 14.0 nM, about 14.5 nM, about 15.0 nM, about 15.5 nM, about 16.0 nM, about 16.5 nM, about 17.0 nM, about 17.5 nM, about 18.0 nM, about 18.5 nM, about 19.0 nM, about 19.5 nM, or about 20.0 nM, or any value therebetween. The Kp is particularly as measured by surface plasmon resonance. In aspects, the antibody may have a Kp to the GI tract expressed antigen of less than about 100 nM, less than about 90 nM, less than about 80 nM, less than about 70 nM, less than about 60 nM, less than about 55 nM, less than about 40 nM, less than about 45 nM, less than about 40 nM, less than about 35 nM, less than about 30 nM, less than about 25 nM, less than 20 nM, less than about 15 nM, less than about 10 nM, less than about 9 nM, less than about 8 nM, less than about 7 nM, less than about 6 nM, less than about 5 nM, less than about 4 nM, less than about 3 nM, less than 2 nM, less than 1 nM. The Kp is particularly as measured by surface plasmon resonance.

The GI tract expressed antigen can be any suitable antigen that is expressed along the GI tract. In aspects, the GI tract expressed antigen is Mucosal Addressin-Cell Adhesion Molecule 1 (MAdCAM-1). For further clarity, the GI tract expressed antigen referred to herein may also represent an in vitro application when said antigen, such as MAdCAM-1, is used to coat wells of a plate, for example, for an in vitro assay (see, for example, Example 2). When the GI tract expressed antigen is MAdCAM-1, the antibody is an anti-MAdCAM-1 antibody. In typical aspects, the antibody described herein is an antibody that binds to human MAdCAM-1, such that the antibody is a human anti-MAdCAM-1 antibody. In further typical aspects, the anti-MAdCAM-1 antibody is a monoclonal antibody, and most typically, a human monoclonal antibody.

The antibody can be a blocking antibody wherein, for example, the function of MAdCAM-1 is inhibited by the antibody described herein. Thus, in aspects, the antibody described herein is useful as a blocking antibody. In aspects, the antibody described herein may be used for regulating the production of immunological cells, such as T cells. In typical aspects, the T cells are of the CD4+ phenotype, but in other aspects, the T cells are of the CD8+ T cells phenotype. In typical aspects, the antibody described herein is useful for expanding populations of CD45RA⁻CD27⁻ T cells, in vitro or in vivo. Thus, in aspects, the antibody preferential expands CD45RA⁻CD27⁻ T cells.

The antibodies described herein may be made by any suitable means known in the art. Human antibodies are described generally in van Dijk and van de Winkel, Curr. Opin. Pharmacol. 5:368-74 (2001) and Lonberg, Curr. Opin. Immunol. 20:450-459 (2008). Monoclonal antibodies can be synthesized by the hybridoma culture or recombinantly, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2.sup.nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625, 126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

Polynucleotides encoding the antibodies described herein are also provided. The polynucleotide can comprise or consist of any of the below provided heavy and light chain polynucleotide sequences for each of clones 11C3, 15D6, 7A2, 2F3, 1B10, 13C5, 15A3, or 6A3, or the below provided HCDR1-3 and LCDR1-3 polynucleotide sequences, for each of the clones 11C3, 15D6, 7A2, 2F3, 1B10, 13C5, 15A3, or 6A3.

Heavy and Light chain DNA sequences of the clones:

11C3 HEAVY CHAIN
(SEQ ID NO: 64)
ATGCTGTTGGGGCTGAAGTGGGTTTTCTTTGTTGTTTTTTATCAAGGTGTGCATTGTGAGGT
AAAACTTGTTGAGTCTGGTGGAGGATTGGTGCAGCCTAAAGGGTCATTGAAACTCTCATGT
GCAGCCTCTGGATTCACCTTCAATACCTACGCCATGAACTGGATCCGCCAGGCTCCAGGA
AAGGGTTTGGAATGGGTTGCTCGCATAAGAAGTAAAAATAATTATTATGCAACATATTATGC
CGATTCAGTGAAAGACAGGTTCACCATCTCCAGAGATGATTCACAAAGTATGCTCTATCTG
CAAATGAACAACTTGAAAACTGAGGACACAGCCATGTATTACTGTGTGAGCTTTGCTTACTG
GGGCCAAGGGACTCTGGTCACTGTCTCTGCA
11C3 LIGHT CHAIN
(SEQ ID NO: 65)
ATGAGGTTCTCTGCTCAGCTTCTGGGGCTGCTTGTGCTCTGGATCCCTGGATCCACTGCA
GAAATTGTGATGACGCAGGCTGCATTCTCCAATCCAGTCACTCTTGGAACATCAGCTTCCA
TCTCCTGCAGGTCTAGTAAGAGTCTCCTACATAGTAATGGCATCACTTATTTGTATTGGTAT
CTGCAGAAGCCAGGCCAGTCTCCTCGGTTCCTGATTTATCAGATGTCCAACCTTGCCTCAG
GAGTCCCAGACAGGTTCACTAGCAGTGGTTCAGGAACTGATTTCACACTGAGAATCAGCAG
AGTGGAGGCTGAGGATGTGGGTGTTTATTACTGTGCTCAAAATCTAGAACTTCCGTGGACG
TTCGGTGGAGGCACCAAGCTGGAAATCAAA
15D6 HEAVY CHAIN
(SEQ ID NO: 66)
ATGGACTCCAGGCTCAATTTAGTTTTCCTTGTCCTTATTTTAAAAGGTGTCCAGTGTGATGT
GCAGCTGGTGGAGTCTGGGGGAGGCTTAGTGCAGCCTGGAGGGTCCCGGAAACTCTCCT
GTGCAGCCTCTGGATTCACTTTCAGCACCTTTGGAATGCACTGGGTTCGTCAGGCTCCAGA
GAAGGGGCTGGAGTGGGTCGCATACATCAGTAGTGGCAGTAGTTCCATCTACTATGCAGA
CACAGTGAAGGGCCGATTCACCATTTCCAGAGACAATCCCGTGAACACCCTGTTCCTGCAA
ATGACCAGTCTAAGGTCTGAAGACACGGCCATGTATTACTGTGCAAGATCCGGACTCGGTA
CTAGCCCCCATGCTATGGACTACTGGGGTCAAGGAACCTCAGTCACCGTCTCCTCA
15D6 LIGHT CHAIN
(SEQ ID NO: 67)
ATGGATTTTCAAGTGCAGATTTTCAGCTTCCTGCTAATGAGTGCCTCAGTCATAATGTCCAG
GGGACAAATTGTTCTCACCCAGTCTCCAACACTCATGTCTGCATCTCCAGGGGAGAAGGTC
ACCATGACCTGCAGTGCCAGCTCAAGTGTAGGTTACATGTACTGGTACCAGCAGAAGCCA
AGATCCTCCCCCAAACCCTGGATTTATCTCACATCCATCCTGGCTTCTGGAGTCCCTGCTC
GCTTCAGTGGCAGTGGGTCTGGGACCTCTTACTCTCTCACAATCAGCAGCATGGAGGCTG
AAGATGCTGCCACTTATTACTGCCAGCAGTGGATTAGTAACCCGCTCACGTTCGGTGCTGG
GACCAAGCTGGAGCTGAAA
7A2 HEAVY CHAIN
(SEQ ID NO: 68)
ATGGCTGTCCTGGTGCTGTTCCTCTGCCTGGCTGCATTTCCAAGCTATGTCCTGTCCCAGG
TGCAGATGGAGCAGTCAGGACCTGGCCTGGTGGCGCCCTCACAGAGCCTGTCCATCACTT
GCACTGTCTCTGACTTTTCACTAATTAACTATGGTGTTCACTGGGTTCGCCAGTCTCCAGGA
AAGGGTCTGGAGTGGCTGGGAGTCATTTGGACTGGTGGAAATACAGAATATAATTCGGCT
CTCATGTCCAGATTGAGCATCACCAAAGACAACTCCAAGAGCCAAGTTTTCTTAAAAATGAA
CAGTCTGCAAGCTGATGACACAGCCATGTACTACTGTGCCCGAGATGACTATTACTTCGGT
GGTGGGGCTATGGACTACTGGGGTCAAGGAACCTCAGTCACCGTCTCCTCA
7A2 LIGHT CHAIN
(SEQ ID NO: 69)
ATGGTATCCACACCTCAGTTCCTTGTATTTTTGCTTTTCTGGATTCCAGCCTCCAGAGGCGA
CATCTTGTTGACTCAGTCTCCAGCCATCCTGTCTGTGAGTCCAGGAGAAAGAGTCAGTTTC
GCCTGCAGGGCCAGTCAGACCATTGGCACAAACATACACTGGTATCAACAACGAACAAAT
GGTTCTCCAAGGCTTCTCATAAAATATGCTTCTGAGTCTATCTCTGAGATCCCTTCCAGGTT
TAGTGGCAGTGGATCGGGGACAGATTTTATTCTCACCATCAGCAGTGTGGAGTCTGAAGAT
ATTGCAGATTATTACTGTCAACAAAGCAATAACTGGCCGCTCACGTTCGGTGCTGGGACCA
AGCTGGAGCTGAGA
2F3 HEAVY CHAIN
(SEQ ID NO: 70)
ATGGAATGGAGATGGATCTTTCTCCTCCTCCTGTCAGGAACTACAGGTGTCCACTCTGAGA
TCCAGCTGCAGCAGTCTGGACCTGAGCTGGTGAAGCCTGGGGCTTCAGTGAAGGTTTCCT
GCAAGGCTTCTGGTTATACATTCACTAGCTTCACCTTGTACTGGGTGAAGCAGAGCCATGG
AAAGAGCCTTGAGTGGATTGGATATATTGATCCTTCCAATGGTGGTACTACCTACAACCAG
AGGTTCAAGGGCAAGGCCACATTGACTGTTGACAAGTCCTCCACCACAGCCTACATGCATC
TCAACAGCCTGACATCTGAGGACTCTTCAGTCTATTACTGTGCAAGAGGAATTTATGATGGT
TACTACGTAGGGAAAATTTTTGACTACTGGGGCCAGGGCACCACTCTCACAGTCTCCTCA
2F3 LIGHT CHAIN
(SEQ ID NO: 71)
ATGGAGACAGACACACTCCTGCTATGGGTGCTGCTGCTCTGGGTTCCAGGTTCCACAGGT
AACATTGTACTGACCCAATCTCCAACTTCTTTGGCTGTGTCTCTCGGGCAGAGGGCCACCA
TATCCTGCAGAGCCAGTGAAAGTGTTGATAATTATGGCAATAGTTTTATGCACTGGTACCAG
CAGAAACCAGGACAGCCACCCAGACTCCTCATCTATCTTGCGTCCAACCTAGAATCTGGG
GTCCCTGCCAGGTTCAGTGGCAGTGGGTCTAGGACAGACTTCACCCTCACCATTGATCCT
GTGGAGGCTGATGATGCTGCAACCTATTACTGTCAGCAAAATAATGAGGATCCTCCGACGT
TCGGTGGAGGCACCAAGCTGGAAATCAGA
1B10 HEAVY CHAIN
(SEQ ID NO: 72)
ATGGGATGGAGCTACATCATCCTCTTTTTGGTAGCAACAGCTACAGATGTCCACTCCCAGG
TCCAACTGCAGCAGCCTGGGGCTGAACTGGTGAAGCCTGGGGCTTCAGTGAAGCTGTCCT
GTAAGGCTTCTGGCTACACCTTCACCAGTTATTGGATGGACTGGGTGAAGCAGAGGCCTG
GACAAGGCCTTGAGTGGATTGGAGAGATTTATCCTAGCAACGGTCGTACTAAGTATAATGA
GAAGTTCAAGAACAAGGCCACACTGACTGTAGACAATTCCTCCAGGACAGCCTACATGCAT
CTAAGCAGCCTGACATCTGAGGACTCTGCGGTCTATTACTGTGCAAGAGGGGGCTATGAT
GTTTACTACGTCGGCAATACTTTGGACTACTGGGGTCAAGGAACCTCAGTCACCGTCTCCTCA
1B10 LIGHT CHAIN
(SEQ ID NO: 73)
ATGGAGACAGACACACTCCTGCTATGGGTGCTGCTGCTCTGGGTTCCAGGTTCCACAGGT
AACATTGTGTTGACCCAATCTCCAGCTTCTTTGGCTGTGTCTCTGGGGCAGAGGGCCACCA
TATCCTGCAGAGCCAGTGAAAGTATTGATAGTTATGGCAATAGTTTTATGCACTGGTACCAC
CAGAAACCAGGACAGCCACCCAAACTCCTCATTTATCTTGCATCCAACCTAGAATCTGGGG
TCCCTGCCAGGTTCAGTGGCAGTGGGTCTAGGACAGACTTCACCCTCACCATTGATCCTGT
GGAGGCTGATGATGCTGCCACCTATTACTGTCTCCAAAATAATGGGGATCCCTGGACGTTC
GGTGGAGGCACCCGGCTGGAGATCAAA
13C5 HEAVY CHAIN
(SEQ ID NO: 74)
ATGGAATGTAACTGGATACTTCCTTTTATTCTGTCGGTAACTTCAGGGGTCTACTCAGAGGT
TCAGCTCCAGCAGTCTGGGACTGTGCTGGCAAGGCCTGGGGCTTCAGTGAAGATGTCCTG
CAAGGCTTCTGGCTACACCTTTACCAGCTACTGGATGCACTGGGTAAAACAGAGGCCTGG
ACAGGGTCTGGAATGGATTGGCGCTATTTATCCTGGAAATAGTGATACTAGCTACAACCAG
AAGTTCAAGGGCAAGGCCAAACTGACTGCAGTCACATCCACCAGCACTGCCTACATGGAG
CTCAGCAGCCTGACAAATGAGGACTCTGCGGTCTATTACTGTACAATCTATGGTAACTACG
GGTACTTCGATGTCTGGGGCGCAGGGACCACGGTCACCGTCTCCTCA
13C5 LIGHT CHAIN
(SEQ ID NO: 75)
ATGGATTCACAGGCCCAGGTTCTTATGTTACTGCTGCTATGGGTATCTGGTACCTGTGGGG
ACATTGTGATGTCACAGTCTCCATCCTCCCTAGCTGTGTCAGTTGGAGAGAAGGTTACTAT
GAGCTGCAAGTCCAGTCAGAGCCTTTTATATAGTAGCAATCAAAAGAACTACTTGGCCTGG
TACCAGCAGAAACCAGGGCAGTCTCCTAAACTGCTGATTTACTGGGCATCCACTAGGGAAT
CTGGGGTCCCTGATCGCTTCACAGGCAGTGGATCTGGGACAGATTTCACTCTCACCATCA
GCAGTGTGAAGGCTGAAGACCTGGCAGTTTATTACTGTCAGCAATATTATAGCTATCCTCC
GTACACGTTCGGAGGGGGGACCAAGCTGGAAATAAAA
15A3 HEAVY CHAIN
(SEQ ID NO: 76)
ATGGAATGGAGGATCTTTCTCTTCATCCTGTCAGGAACTGCATGTGTCCACTCCCAGGATC
AGCTGCTGCAGTCTGGGCCTGAACTGGTGAAGCCTGGGACTTCAGTGAAGATGTCCTGCA
GGGCTTCTGGATACACATTCAGTGACGATGTCATAAACTGGGTGCGGAAGAGAACTGGAC
AGGGCCTGGAATGGATTGGAGAGATTTATCCTAGAATTGGTAGTATGTACTACAATGAGAA
TTTCAAGGGCAGGGCCACTCTGACTGCAGACAAATCGTCCAACACAGTCTACATTCATCTC
AGCAGCCTGACATCTGAGGACTCTGCGGTCTATTTCTGCGCACGATATCTACTAACGAAGG
GCTATGGTATGGACTACTGGGGTCAAGGCACCTCAGTCACCGTCTCCTCA
15A3 LIGHT CHAIN
(SEQ ID NO: 77)
ATGGAGACAGACACACTCCTGTTATGGGTACTGCTGCTCTGGGTTCCAGGTTCCACTGGT
GACATTGTGCTGACACAGTCTCCTGCTTCCTTAGCTGTGTCTCTGGGGCAGAGGGCCACC
ATCTCATGCAGGGCCAGCAAAAGTGTCAGTCCATCTGACTATAGTTATATCCACTGGTACC
AACAGAAACCAGGACAGCCACCCAAACTCCTCATCTATCTTGCATCCAACCTAGAATCTGG
GGTCCCTGCCAGGTTCAGTGGCAGTGGGTCTGGGACAGACTTCACCCTCAACATCCATCC
TGTGGAGGAGGAGGATGCTGCAACCTATTACTGTCAACACAGTAGGGAGCTTCCTCGGAC
GTTCGGTGGAGGCACCAAGTTGGAAATCAAA
6A3 HEAVY CHAIN
(SEQ ID NO: 78)
ATGGCTGTCTTGGGGCTGCTCTTCTGCCTGGTGACATTCCCAAGCTGTGTCCTATCCCAGG
TGCAGCTGAAACAGTCAGGACCTGGCCTAGTGCAGCCCTCACAGAGCCTGTCCATCACCT
GCACCGTCTCTGGTTTCTCATTAACTAGCTATGGTTTACACTGGGTTCGCCAGTCTCCAGG
AAAGGGTCTGGAGTGGTTGGGATTCATTTGGAGTGGTGGAGCCACAGACTATAATGCAGC
TTTCATATCCAGACTGAGCATCAGCAAGGACAATTCCAAGAGGCAAGTTTTCTTTAAAATGA
ACAGTCTGCAAGCTAATGACACAGCCATATATTTCTGTGCCAGAAGGGATGATAATTACGC
CTTGGCTATGGACTACTGGGGTCAAGGAACCTCAGTCACCGTCTCCTCA
6A3 LIGHT CHAIN
(SEQ ID NO: 79)
ATGGATTTTCAAGTGCAGATTTTCAGCTTCCTGCTAATGAGTGCCTCAGTCATAATGTCCAG
GGGACAAATTGTTCTCACCCAGTCTCCAGCACTCATGTCTGCATCTCCAGGGGAGAAGGT
CACCATGACCTGCCGTGCCAGCTCAAGTGTAAGTTACATATACTGGTACCAGCAGAAGCCA
AGATCCTCCCCCAAACCCTGGATTTATCTCACATCCGACCTGGCTTCTGGAGTCCCTACTC
GCTTCAGTGGCAGTGGGTCTGGGACCTCTTACTTTCTTACAATCAGTAGCATGGAGGCTGA
AGATACTGCCACTTATTACTGCCAGCAGTGGAATAGTAATCCGCTCACATTCGGTGCTGGG
ACCAAGCTGGAACTGAAA
HCDR and LCDR DNA sequences of the clones:
11C3 HEAVY CHAIN CDR 1
(SEQ ID NO: 80)
ACCTACGCCATGAAC
11C3 LIGHT CHAIN CDR 1
(SEQ ID NO: 81)
AGGTCTAGTAAGAGTCTCCTACATAGTAATGGCATCACTTATTTGTAT
11C3 HEAVY CHAIN CDR 2
(SEQ ID NO: 82)
CGCATAAGAAGTAAAAATAATTATTATGCAACATATTATGCCGATTCAGTGAAAGAC
11C3 LIGHT CHAIN CDR 2
(SEQ ID NO: 83)
CAGATGTCCAACCTTGCCTCA
11C3 HEAVY CHAIN CDR 3:
TTTGCTTAC
11C3 LIGHT CHAIN CDR 3
(SEQ ID NO: 84)
GCTCAAAATCTAGAACTTCCGTGGACG
15D6 HEAVY CHAIN CDR 1
(SEQ ID NO: 85)
ACCTTTGGAATGCAC
15D6 LIGHT CHAIN CDR 1
(SEQ ID NO: 86)
AGTGCCAGCTCAAGTGTAGGTTACATGTAC
15D6 HEAVY CHAIN CDR 2
(SEQ ID NO: 87)
TACATCAGTAGTGGCAGTAGTTCCATCTACTATGCAGACACAGTGAAGGGC
15D6 LIGHT CHAIN CDR 2
(SEQ ID NO: 88)
CTCACATCCATCCTGGCTTCT
15D6 HEAVY CHAIN CDR 3
(SEQ ID NO: 89)
TCCGGACTCGGTACTAGCCCCCATGCTATGGACTAC
15D6 LIGHT CHAIN CDR 3
(SEQ ID NO: 90)
CAGCAGTGGATTAGTAACCCGCTCACG
7A2 HEAVY CHAIN CDR 1
(SEQ ID NO: 91)
AACTATGGTGTTCAC
7A2 LIGHT CHAIN CDR 1
(SEQ ID NO: 92)
AGGGCCAGTCAGACCATTGGCACAAACATACAC
7A2 HEAVY CHAIN CDR 2
(SEQ ID NO: 93)
GTCATTTGGACTGGTGGAAATACAGAATATAATTCGGCTCTCATGTCC
7A2 LIGHT CHAIN CDR 2
(SEQ ID NO: 94)
TATGCTTCTGAGTCTATCTCT
7A2 HEAVY CHAIN CDR 3
(SEQ ID NO: 95)
GATGACTATTACTTCGGTGGTGGGGCTATGGACTAC
7A2 LIGHT CHAIN CDR 3
(SEQ ID NO: 96)
CAACAAAGCAATAACTGGCCGCTCACG
2F3 HEAVY CHAIN CDR 1
(SEQ ID NO: 97)
AGCTTCACCTTGTAC
2F3 LIGHT CHAIN CDR 1
(SEQ ID NO: 98)
AGAGCCAGTGAAAGTGTTGATAATTATGGCAATAGTTTTATGCAC
2F3 HEAVY CHAIN CDR 2
(SEQ ID NO: 99)
TATATTGATCCTTCCAATGGTGGTACTACCTACAACCAGAGGTTCAAGGGC
2F3 LIGHT CHAIN CDR 2
(SEQ ID NO: 100)
CTTGCGTCCAACCTAGAATCT
2F3 HEAVY CHAIN CDR 3
(SEQ ID NO: 101)
GGAATTTATGATGGTTACTACGTAGGGAAAATTTTTGACTAC
2F3 LIGHT CHAIN CDR 3
(SEQ ID NO: 102)
CAGCAAAATAATGAGGATCCTCCGACG
1B10 HEAVY CHAIN CDR 1
(SEQ ID NO: 103)
AGTTATTGGATGGAC
1B10 LIGHT CHAIN CDR 1
(SEQ ID NO: 104)
AGAGCCAGTGAAAGTATTGATAGTTATGGCAATAGTTTTATGCAC
1B10 HEAVY CHAIN CDR 2
(SEQ ID NO: 105)
GAGATTTATCCTAGCAACGGTCGTACTAAGTATAATGAGAAGTTCAAGAAC
1B10 LIGHT CHAIN CDR 2
(SEQ ID NO: 106)
CTTGCATCCAACCTAGAATCT
1B10 HEAVY CHAIN CDR 3
(SEQ ID NO: 107)
GGGGGCTATGATGTTTACTACGTCGGCAATACTTTGGACTAC
1B10 LIGHT CHAIN CDR 3
(SEQ ID NO: 108)
CTCCAAAATAATGGGGATCCCTGGACG
13C5 HEAVY CHAIN CDR 1
(SEQ ID NO: 109)
AGCTACTGGATGCAC
13C5 LIGHT CHAIN CDR 1
(SEQ ID NO: 110)
AAGTCCAGTCAGAGCCTTTTATATAGTAGCAATCAAAAGAACTACTTGGCC
13C5 HEAVY CHAIN CDR 2
(SEQ ID NO: 111)
GCTATTTATCCTGGAAATAGTGATACTAGCTACAACCAGAAGTTCAAGGGC
13C5 LIGHT CHAIN CDR 2
(SEQ ID NO: 112)
TGGGCATCCACTAGGGAATCT
13C5 HEAVY CHAIN CDR 3
(SEQ ID NO: 113)
TATGGTAACTACGGGTACTTCGATGTC
13C5 LIGHT CHAIN CDR 3
(SEQ ID NO: 114)
CAGCAATATTATAGCTATCCTCCGTACACG
15A3 HEAVY CHAIN CDR 1
(SEQ ID NO: 115)
GACGATGTCATAAAC
15A3 LIGHT CHAIN CDR 1
(SEQ ID NO: 116)
AGGGCCAGCAAAAGTGTCAGTCCATCTGACTATAGTTATATCCAC
15A3 HEAVY CHAIN CDR 2
(SEQ ID NO: 117)
GAGATTTATCCTAGAATTGGTAGTATGTACTACAATGAGAATTTCAAGGGC
15A3 LIGHT CHAIN CDR 2
(SEQ ID NO: 118)
CTTGCATCCAACCTAGAATCT
15A3 HEAVY CHAIN CDR 3
(SEQ ID NO: 119)
TATCTACTAACGAAGGGCTATGGTATGGACTAC
15A3 LIGHT CHAIN CDR 3
(SEQ ID NO: 120)
CAACACAGTAGGGAGCTTCCTCGGACG
6A3 HEAVY CHAIN CDR 1
(SEQ ID NO: 121)
AGCTATGGTTTACAC
6A3 LIGHT CHAIN CDR 1
(SEQ ID NO: 122)
CGTGCCAGCTCAAGTGTAAGTTACATATAC
6A3 HEAVY CHAIN CDR 2
(SEQ ID NO: 123)
TTCATTTGGAGTGGTGGAGCCACAGACTATAATGCAGCTTTCATATCC
6A3 LIGHT CHAIN CDR 2
(SEQ ID NO: 124)
CTCACATCCGACCTGGCTTCT
6A3 HEAVY CHAIN CDR 3
(SEQ ID NO: 125)
AGGGATGATAATTACGCCTTGGCTATGGACTAC
6A3 LIGHT CHAIN CDR 3
(SEQ ID NO: 126)
CAGCAGTGGAATAGTAATCCGCTCACA

Also described herein are constructs comprising the antibody described herein. In aspects, the construct comprises the antibody and a cargo molecule. The antibody is capable of binding to GI tract expressed antigen. In this way, the construct may be targeted to the GI tract expressed antigen. In other aspects, the binding allows for the antibody to preferentially expand T cells, in particular, CD45RA⁻CD27⁻ T cells. The constructs described herein are also referred to as bispecific constructs, and the terms may be used interchangeably throughout. For the sake of brevity, any description of the antibody and the GI tract expressed antigen will not be repeated as they are found in the section above and are equally applicable here.

The construct may comprise the antibody as a full length antibody or the construct may comprise the antibody as a functional antibody fragment. The term “full length antibody” includes the structure that constitutes the natural biological form of an antibody, including variable and constant regions. By functional antibody fragment, it meant that the antibody comprises at least the portion of the antibody that can bind to the GI tract expressed antigen. In typical aspects, the antibody is a functional antibody fragment that is capable of binding to, or that binds to, the GI tract expressed antigen described herein.

The functional antibody fragment and cargo molecule may be connected to each other by any suitable means, including, but not limited to recombinant technology, chemical conjugation, chelation, and the like. In typical aspects, the functional antibody fragment is fused to the cargo molecule. The functional antibody fragment and the cargo molecule may be connected to one another via a linker, and typically the linker has the sequence GGGGGGGGSGGGGS (SEQ ID NO: 127).

As with the antibody described herein, the functional antibody fragment is capable of binding to, or binds to, the GI tract expressed antigen, and in typical aspects, the cargo molecule does not impact, impair, lessen, or reduce, the ability of the functional antibody fragment to bind to the GI tract expressed antigen. In this way, the construct is able to be targeted to the GI tract expressed antigen and, for example, the cargo molecule may be able to accumulate at a targeted site in the GI tract. In aspects, the targeted site in the GI tract comprises the small intestine, the large intestine and/or the colon. This may assist with limiting off-target effects of the cargo molecule since the construct is targeted to the GI tract, and in specific aspects, the targeted site in the GI tract. Similar to the description of the antibody described herein, the functional antibody fragment has a Kp of between about 0.05 nM to about 20 nM for the GI tract expressed antigen.

The functional antibody fragment can be any fragment that is capable of binding to, or that retains the ability to bind to, the GI tract expressed antigen. The functional antibody fragment can be, but is not limited to, for example, a scFv, a Fab′, a Fab, a F(ab′) 2, a Fv, or a scFab. In typical aspects, the functional antibody fragment is a scFv. In aspects, the scFv comprises or consists of a polypeptide having the sequence of:

(SEQ ID NO: 128)
QVQMEQSGPGLVAPSQSLSITCTVSDFSLINYGVHWVRQSPGKGLEWLG
VIWTGGNTEYNSALMSRLSITKDNSKSQVFLKMNSLQADDTAMYYCARD
DYYFGGGAMDYWGQGTSVTVSSGGGGSGGGGSGGGGSDILLTQSPAILS
VSPGERVSFACRASQTIGTNIHWYQQRTNGSPRLLIKYASESISEIPSR
FSGSGSGTDFILTISSVESEDIADYYCQQSNNWPLTFGAGTKLELRAAA
YPYDVPDYAHHHHHH

In aspects, the scFv comprises or consists of a polypeptide having the sequence of:

(SEQ ID NO: 129)
ATMETDTLLLWVLLLWVPGSTGQVQMEQSGPGLVAPSQSLSITCTVSDF
SLINYGVHWVRQSPGKGLEWLGVIWTGGNTEYNSALMSRLSITKDNSKS
QVFLKMNSLQADDTAMYYCARDDYYFGGGAMDYWGQGTSVTVSSGGGGS
GGGGSGGGGSDILLTQSPAILSVSPGERVSFACRASQTIGTNIHWYQQR
TNGSPRLLIKYASESISEIPSRFSGSGSGTDFILTISSVESEDIADYYC
QQSNNWPLTFGAGTKLELRAAAYPYDVPDYAHHHHHH

where underling represented an optional secretion signal (SEQ ID NO: 130).

In further aspects, the scFv consists of VL and VH of the antibody, wherein the VL and VH are connected by a peptide linker. The peptide linker can enable the scFv to form the desired structure for binding to the GI expressed antigen (see, for example, Pluckthun, The Pharmacology of Monoclonal Antibodies, vol. 113, Rosenburg and Moore eds., Springer-Verlag, New York, 1994, pp. 269-315, incorporated by reference herein). While any suitable peptide linker may used, in typical aspects, the peptide linker comprises the sequence GGGGSGGGGSGGGGS (SEQ ID NO: 127). The construct may also further comprise a detection tag, such as a histidine tag, which is normally at the C-terminus of the construct.

It will be understood that the functional antibody fragment, such as the scFv described herein, may be at or near the N-terminus or the C-terminus of the construct and the cargo molecule would be at or near the corresponding C-terminus or N-terminus. Typically, however, the functional antibody fragment, such as the scFv described herein, is at or near the N-terminus of the construct and the cargo molecule is at or near the C-terminus of the construct.

In aspects, the scFv is capable of binding to, or binds to, the GI tract expressed antigen. As described for the antibody described herein, the GI tract expressed antigen may be Mucosal Addressin-Cell Adhesion Molecule 1 (MAdCAM-1). In this way, when the GI tract expressed antigen is MAdCAM-1, and the scFv is capable of binding to, or binds to, MAdCAM-1, the scFv may be referred to as an anti-MAdCAM-1 scFv.

The construct may comprise any suitable cargo. The cargo molecule may be selected from, but not limited to, for example, proteins, peptides, nucleic acids, amino acids, nucleosides, antibodies, antibody fragments, antibody ligands, peptide nucleic acids, small organic molecules, lipids, hormones, drugs, enzymes, lectin, cell adhesion molecule, antibody epitope, enzyme substrates, enzyme inhibitors, coenzymes, inorganic molecules, carbohydrates, such as polysaccharides and monosaccharides, or a combination thereof. In typical aspects, the cargo molecule is a protein.

The protein may be a cytokine, such as IFNα, IFNβ, IFNγ, TNF-α, TNF-β, TGF-β, IL-1α, IL-1β, IL-2, IL-3, IL-4, IL-6, IL-8, IL-10, IL-12β, IL-13, IL-17, II-18, IL-22, IL-23, and, GM-CSF, G-CSF, M-CSF, and the like. The protein may also be a chemokine, such as, CCL1, CCL2, CCL3, CCL4, CCL5, CCL2, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, and the like. In typical aspects, the protein is a cytokine, and the cytokine is typically IL-22. In other aspects, the cargo molecule is a protein that has cytokine blocking functions, like IL-18 bp, which antagonizes the function of IL-18, or an IL-23 blocker, such as Ustekinumab or Risankizumab.

When the cargo is a cytokine, and the cytokine is IL-22, the construct described herein can have the sequence:

(SEQ ID NO: 131)
QVQMEQSGPGLVAPSQSLSITCTVSDFSLINYGVHWVRQSPGKGLEWLG
VIWTGGNTEYNSALMSRLSITKDNSKSQVFLKMNSLQADDTAMYYCARD
DYYFGGGAMDYWGQGTSVTVSSGGGGSGGGGSGGGGSDILLTQSPAILS
VSPGERVSFACRASQTIGTNIHWYQQRTNGSPRLLIKYASESISEIPSR
FSGSGSGTDFILTISSVESEDIADYYCQQSNNWPLTFGAGTKLELRGGG
GGGGGSGGGGSGGGGSGGGGSAPISSHCRLDKSNFQQPYITNRTFMLAK
EASLADNNTDVRLIGEKLFHGVSMSERCYLMKQVLNFTLEEVLFPQSDR
FQPYMQEVVPFLARLSNRLSTCHIEGDDLHIQRNVQKLKDTVKKLGESG
EIKAIGELDLLFMSLRNACIAAAYPYDVPDYAHHHHHH.

In this specific aspect, the construct does not have the secretion sequence (SEQ ID NO: 130).

In other specific aspects, When the cargo is a cytokine, and the cytokine is IL-22, the construct described herein can have the sequence:

(SEQ ID NO: 132)
ATMETDTLLLWVLLLWVPGSTGQVQMEQSGPGLVAPSQSLSITCTVSDF
SLINYGVHWVRQSPGKGLEWLGVIWTGGNTEYNSALMSRLSITKDNSKS
QVFLKMNSLQADDTAMYYCARDDYYFGGGAMDYWGQGTSVTVSSGGGGS
GGGGSGGGGSDILLTQSPAILSVSPGERVSFACRASQTIGTNIHWYQQR
TNGSPRLLIKYASESISEIPSRFSGSGSGTDFILTISSVESEDIADYYC
QQSNNWPLTFGAGTKLELRGGGGGGGGSGGGGSGGGGSGGGGSAPISSH
CRLDKSNFQQPYITNRTFMLAKEASLADNNTDVRLIGEKLFHGVSMSER
CYLMKQVLNFTLEEVLFPQSDRFQPYMQEVVPFLARLSNRLSTCHIEGD
DLHIQRNVQKLKDTVKKLGESGEIKAIGELDLLFMSLRNACIAAAYPYD
VPDYAHHHHHH

where the underlining represents the optional secretion signal (SEQ ID NO: 130)

In aspects, the construct described herein is capable of at least one of the following: (a) allowing for inhibition of IL-18; (b) allowing for production of IL-10; (c) inducing STAT3 phosphorylation; and (d) reducing IFNγ secretion. In typical aspects, these functions of the constructs are when the construct has been delivered to the GI expressed antigen. It would be understood that these functions are dependent on the identity of the cargo molecule as well. For example, the construct comprising the cargo molecule of IL-18 bp is capable of (a), whereas the construct comprising the cargo molecule of IL-22 is capable of (b)-(d).

The constructs described herein may also comprise additional sequences to aid in their expression, detection or purification. Any such sequences or tags known to those of skill in the art may be used. For example, and without wishing to be limiting, the constructs may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK (SEQ ID NO: 133), Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a Hiss or His6), or a combination thereof. In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv or scFab) to an effector domain, (e) interposed between and connecting subunits of a binding domain, or (f) at the amino-terminus of a single chain fusion protein. In certain aspects, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

Of note and with references to the sequences provided herein, sequences that are substantially identical to the sequences for the antibodies and the constructs described herein are also contemplated, such as those that are at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical. Fragments of the sequences or the substantially identical variant sequences are also contemplated herein.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

Also described herein are nucleic acid molecules encoding the constructs described herein, as well as vectors comprising the nucleic acid molecules and host cells comprising the vectors.

For example, a polynucleotide encoding the construct described herein, in aspects, has the sequence:

(SEQ ID NO: 134)
GCCACCATGGAAACCGACACACTGCTGCTGTGGGTGCTGCTTCTTTGGG
TGCCCGGATCTACAGGCCAGGTGCAGATGGAACAGTCTGGCCCTGGACT
GGTGGCCCCATCTCAGAGCCTGAGCATCACCTGTACCGTGTCCGACTTC
AGCCTGATCAACTACGGCGTGCACTGGGTCCGACAGAGCCCTGGAAAAG
GACTGGAATGGCTGGGCGTGATCTGGACCGGCGGCAACACAGAGTACAA
CAGCGCCCTGATGAGCCGGCTGTCCATCACCAAGGACAACAGCAAGAGC
CAGGTGTTCCTGAAGATGAACTCCCTGCAGGCCGACGACACCGCCATGT
ACTACTGCGCCAGAGATGACTACTACTTCGGCGGAGGCGCCATGGATTA
TTGGGGCCAGGGAACAAGCGTGACCGTGTCTAGCGGAGGCGGAGGATCT
GGTGGCGGAGGTAGTGGTGGCGGCGGATCTGATATTCTGCTGACACAGT
CCCCTGCCATCCTGTCCGTTTCTCCAGGCGAGAGAGTGTCCTTCGCCTG
TAGAGCCTCTCAGACCATCGGCACCAACATCCACTGGTATCAGCAGCGG
ACCAACGGCAGCCCCAGACTGCTGATTAAGTACGCCAGCGAGAGCATCA
GCGAGATCCCCAGCAGATTTTCTGGCAGCGGCTCCGGCACCGATTTCAT
CCTGACAATCAGCTCCGTGGAAAGCGAGGATATCGCCGATTACTACTGC
CAGCAGAGCAACAACTGGCCCCTGACATTTGGAGCCGGCACCAAGCTGG
AACTTAGAGGCGGCGGAGGTTCTGGCGGTGGTGGATCTGGCGGAGGTGG
AAGCGGCGGAGGCGGCTCTGGCGGCGGAGGAAGTGCTCCTATTAGCAGC
CACTGCCGGCTGGACAAGAGCAACTTCCAGCAGCCTTACATCACCAACC
GGACCTTCATGCTGGCCAAAGAGGCCAGCCTGGCCGACAACAATACTGA
CGTGCGGCTGATCGGCGAGAAGCTGTTTCATGGCGTGTCCATGAGCGAG
CGGTGCTACCTGATGAAGCAGGTCCTGAACTTCACCCTGGAAGAGGTGC
TGTTCCCTCAGAGCGACCGGTTTCAGCCCTACATGCAAGAGGTGGTGCC
CTTTCTGGCCCGGCTGAGCAATAGACTGAGCACCTGTCACATCGAGGGC
GACGACCTGCACATCCAGAGAAACGTGCAGAAACTGAAGGACACCGTGA
AGAAGCTGGGCGAGAGCGGAGAGATCAAGGCCATCGGAGAACTGGACCT
GCTGTTCATGAGCCTGCGGAACGCCTGTATCGCCGCTGCCTATCCTTAC
GACGTGCCCGATTATGCCCACCACCACCATCACCACTGATGATGA

In another aspect, the polynucleotide encoding the scFv described herein, in aspects, has the sequence:

(SEQ ID NO: 135)
GCCACCATGGAAACCGACACACTGCTGCTGTGGGTGCTGCTTCTTTGGG
TGCCCGGATCTACAGGCCAGGTGCAGATGGAACAGTCTGGCCCTGGACT
GGTGGCCCCATCTCAGAGCCTGAGCATCACCTGTACCGTGTCCGACTTC
AGCCTGATCAACTACGGCGTGCACTGGGTCCGACAGAGCCCTGGAAAAG
GACTGGAATGGCTGGGCGTGATCTGGACCGGCGGCAACACAGAGTACAA
CAGCGCCCTGATGAGCCGGCTGTCCATCACCAAGGACAACAGCAAGAGC
CAGGTGTTCCTGAAGATGAACTCCCTGCAGGCCGACGACACCGCCATGT
ACTACTGCGCCAGAGATGACTACTACTTCGGCGGAGGCGCCATGGATTA
TTGGGGCCAGGGAACAAGCGTGACCGTGTCTAGCGGAGGCGGAGGATCT
GGTGGCGGAGGTAGTGGTGGCGGCGGATCTGATATTCTGCTGACACAGT
CCCCTGCCATCCTGTCCGTTTCTCCAGGCGAGAGAGTGTCCTTCGCCTG
TAGAGCCTCTCAGACCATCGGCACCAACATCCACTGGTATCAGCAGCGG
ACCAACGGCAGCCCCAGACTGCTGATTAAGTACGCCAGCGAGAGCATCA
GCGAGATCCCCAGCAGATTTTCTGGCAGCGGCTCCGGCACCGATTTCAT
CCTGACAATCAGCTCCGTGGAAAGCGAGGATATCGCCGATTACTACTGC
CAGCAGAGCAACAACTGGCCCCTGACATTTGGAGCCGGCACCAAGCTGG
AACTGAGAGCCGCCGCTTATCCCTACGACGTGCCAGACTATGCCCACCA
CCACCATCACCACTGATGATGA

Polynucleotides encoding the constructs described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of polynucleotides that encode fragments of antibodies include any cell, such as hybridomas and spleen cells, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

Additionally, expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as MI3 and other filamentous single-stranded DNA phages. An example of a vector useful in yeast is the 2u plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1:854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The constructs described herein can be expressed in cell lines other than in hybridomas. Nucleic acids, which comprise a sequence encoding a polypeptide, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These recombinant host cells can be used to produce proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60 (6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium. An example of such a sequence may be, for example, ATMETDTLLLWVLLLWVPGSTG (SEQ ID NO: 132).

The constructs described herein can be fused to additional amino acid residues. Such amino acid residues can be a peptide tag to facilitate isolation, for example. Other amino acid residues for homing of the antibodies to specific organs or tissues are also contemplated.

Also described herein are methods and uses of the antibodies and constructs described herein. In aspects, methods of targeting the construct described herein to a GI tract expressed antigen is provided. The method comprises administering the construct to a subject and targeting the construct to the GI tract of the subject. In typical aspects, the subject is a human. The targeting is to a targeted site in the GI tract, and the targeted site in the GI tract comprises the small intestine, the large intestine and/or the colon. Thus, in aspects, the targeted site in the GI tract is the small intestine, in other aspects, the targeted site in the GI tract is the large intestine, and in further aspects, the targeted site in the GI tract is the colon. Targeting is possible via the antibody or functional antibody fragment, both of which are capable of binding, or binds, to the GI tract expressed antigen. Thus, in aspects, the method may further comprise delivering the antibody or construct to the target site of the GI tract. The method may further comprise binding, of the antibody or the construct, to the GI tract expressed antigen.

In aspects, uses of the construct described herein for targeting the construct to the GI tract expressed antigen are provided. In these aspects, the construct is targetable to the GI tract expressed antigen via the antibody or functional antibody, each of which are capable of binding, or binds, to, the GI tract expressed antigen as described herein. In this way, the antibody or construct are targetable or deliverable to the target site of the GI tract.

The GI tract expressed antigen is typically MAdCAM-1. As described above, these methods and uses can allow for accumulation of the cargo molecule in the GI tract, in specific aspects, accumulation at the target site of the GI tract, such that, for example, off-target effects of the cargo molecule may be reduced.

In other aspects, methods and uses related to the preferential expansion of T cells is also provided. Thus, in aspects, methods of preferentially expanding T cells, such as CD4+ and/or CD8+ T cells, are provided. In typical aspects, the T cells are CD45 CD27⁻ T cells. These methods typically comprise binding of the antibody or construct described herein to the GI tract expressed antigen thereby preferentially expanding the T cells. The methods and uses related to T cell expansion may be in respect to in vitro or in vivo applications.

Any suitable method or route can be used to administer the antibodies and constructs described herein. Routes of administration include, for example, oral, intravenous, intraperitoneal, subcutaneous, or intramuscular administration.

It is understood that the antibodies or constructs described herein, can be administered in the form of a composition, such as, for example, a sterile aqueous solution, additionally comprising a pharmaceutically acceptable carrier. Suitable pharmaceutically acceptable carriers include, for example, one or more of water, saline, phosphate buffered saline, dextrose, glycerol, ethanol and the like (see, generally, Remington's Pharmaceutical Sciences 16^{th} Edition, A. Osal., Ed., 1980), as well as combinations thereof. Acceptable carriers, excipients, or stabilizers are nontoxic to recipients at the dosages and concentrations employed and may include buffers. Pharmaceutically acceptable carriers may further comprise minor amounts of auxiliary substances such as wetting or emulsifying agents, preservatives or buffers, which enhance the shelf life or effectiveness of the binding proteins.

Although human antibodies are particularly useful for administration to humans, they may be administered to other mammals as well. The term “mammal” as used herein is intended to include, but is not limited to, humans, laboratory animals, domestic pets and farm animals. Thus, the subject in the methods and uses described herein includes, but is not limited to, humans, laboratory animals, domestic pets and farm animals. In typical aspects, the mammal is a human.

Also included herein are kits, comprising the antibodies and the constructs described herein. The kits can further contain any suitable components, including buffers, for example. Kits may include instructions.

### SEQUENCE LISTING

Sequence listing

11C3 HEAVY CHAIN CDR 1 (SEQ ID NO: 1)
TYAMN
11C3 LIGHT CHAIN CDR 1 (SEQ ID NO: 2)
RSSKSLLHSNGITYLY
11C3 HEAVY CHAIN CDR 2 (SEQ ID NO: 3)
RIRSKNNYYATYYADSVKD
11C3 LIGHT CHAIN CDR 2 (SEQ ID NO: 4)
QMSNLAS
11C3 HEAVY CHAIN CDR 3:
FAY
11C3 LIGHT CHAIN CDR 3 (SEQ ID NO: 5)
AQNLELPWT
15D6 HEAVY CHAIN CDR 1 (SEQ ID NO: 6)
TFGMH
15D6 LIGHT CHAIN CDR 1 (SEQ ID NO: 7)
SASSSVGYMY
15D6 HEAVY CHAIN CDR 2 (SEQ ID NO: 8)
YISSGSSSIYYADTVKG
15D6 LIGHT CHAIN CDR 2 (SEQ ID NO: 9)
LTSILAS
15D6 HEAVY CHAIN CDR 3 (SEQ ID NO: 10)
SGLGTSPHAMDY
15D6 LIGHT CHAIN CDR 3 (SEQ ID NO: 11)
QQWISNPLT
7A2 HEAVY CHAIN CDR 1 (SEQ ID NO: 12)
NYGVH
7A2 LIGHT CHAIN CDR 1 (SEQ ID NO: 13)
RASQTIGTNIH
7A2 HEAVY CHAIN CDR 2 (SEQ ID NO: 14)
VIWTGGNTEYNSALMS
7A2 LIGHT CHAIN CDR 2 (SEQ ID NO: 15)
YASESIS
7A2 HEAVY CHAIN CDR 3 (SEQ ID NO: 16)
DDYYFGGGAMDY
7A2 LIGHT CHAIN CDR 3 (SEQ ID NO: 17)
QQSNNWPLT
2F3 HEAVY CHAIN CDR 1 (SEQ ID NO: 18)
SFTLY
2F3 LIGHT CHAIN CDR 1 (SEQ ID NO: 19)
RASESVDNYGNSFMH
2F3 HEAVY CHAIN CDR 2 (SEQ ID NO:20)
YIDPSNGGTTYNQRFKG
2F3 LIGHT CHAIN CDR 2 (SEQ ID NO: 21)
LASNLES
2F3 HEAVY CHAIN CDR 3 (SEQ ID NO: 22)
GIYDGYYVGKIFDY
2F3 LIGHT CHAIN CDR 3 (SEQ ID NO: 23)
QQNNEDPPT
1B10 HEAVY CHAIN CDR 1 (SEQ ID NO: 24)
SYWMD
1B10 LIGHT CHAIN CDR 1 (SEQ ID NO: 25)
RASESIDSYGNSFMH
1B10 HEAVY CHAIN CDR 2 (SEQ ID NO: 26)
EIYPSNGRTKYNEKFKN
1B10 LIGHT CHAIN CDR 2 (SEQ ID NO: 27)
LASNLES
1B10 HEAVY CHAIN CDR 3 (SEQ ID NO: 28)
GGYDVYYVGNTLDY
1B10 LIGHT CHAIN CDR 3 (SEQ ID NO: 29)
LQNNGDPWT
13C5 HEAVY CHAIN CDR 1 (SEQ ID NO: 30)
SYWMH
13C5 LIGHT CHAIN CDR 1 (SEQ ID NO: 31)
KSSQSLLYSSNQKNYLA
13C5 HEAVY CHAIN CDR 2 (SEQ ID NO: 32)
AIYPGNSDTSYNQKFKG
13C5 LIGHT CHAIN CDR 2 (SEQ ID NO: 33)
WASTRES
13C5 HEAVY CHAIN CDR 3 (SEQ ID NO: 34)
YGNYGYFDV
13C5 LIGHT CHAIN CDR 3 (SEQ ID NO: 35)
QQYYSYPPYT
15A3 HEAVY CHAIN CDR 1 (SEQ ID NO: 36)
DDVIN
15A3 LIGHT CHAIN CDR 1 (SEQ ID NO: 37)
RASKSVSPSDYSYIH
15A3 HEAVY CHAIN CDR 2 (SEQ ID NO: 38)
EIYPRIGSMYYNENFKG
15A3 LIGHT CHAIN CDR 2 (SEQ ID NO: 39)
LASNLES
15A3 HEAVY CHAIN CDR 3 (SEQ ID NO: 40)
YLLTKGYGMDY
15A3 LIGHT CHAIN CDR 3 (SEQ ID NO: 41)
QHSRELPRT
6A3 HEAVY CHAIN CDR 1 (SEQ ID NO: 42)
SYGLH
6A3 LIGHT CHAIN CDR 1 (SEQ ID NO: 43)
RASSSVSYIY
6A3 HEAVY CHAIN CDR 2 (SEQ ID NO: 44)
FIWSGGATDYNAAFIS
6A3 LIGHT CHAIN CDR 2 (SEQ ID NO: 45)
LTSDLAS
6A3 HEAVY CHAIN CDR 3 (SEQ ID NO: 46)
RDDNYALAMDY
6A3 LIGHT CHAIN CDR 3 (SEQ ID NO: 47)
QQWNSNPLT
11C3 HEAVY CHAIN (SEQ ID NO: 48)
MLLGLKWVFFVVFYQGVHCEVKLVESGGGLVQPKGSLKLSCAASGFTFNTYAMNWIRQAPGK
GLEWVARIRSKNNYYATYYADSVKDRFTISRDDSQSMLYLQMNNLKTEDTAMYYCVSFAYWG
QGTLVTVSA
11C3 LIGHT CHAIN (SEQ ID NO: 49)
MRFSAQLLGLLVLWIPGSTAEIVMTQAAFSNPVTLGTSASISCRSSKSLLHSNGITYLYWYLQKP
GQSPRFLIYQMSNLASGVPDRFTSSGSGTDFTLRISRVEAEDVGVYYCAQNLELPWTFGGGTK
LEIK
15D6 HEAVY CHAIN (SEQ ID NO: 50)
MDSRLNLVFLVLILKGVQCDVQLVESGGGLVQPGGSRKLSCAASGFTFSTFGMHWVRQAPEK
GLEWVAYISSGSSSIYYADTVKGRFTISRDNPVNTLFLQMTSLRSEDTAMYYCARSGLGTSPHA
MDYWGQGTSVTVSS
15D6 LIGHT CHAIN (SEQ ID NO: 51)
MDFQVQIFSFLLMSASVIMSRGQIVLTQSPTLMSASPGEKVTMTCSASSSVGYMYWYQQKPRS
SPKPWIYLTSILASGVPARFSGSGSGTSYSLTISSMEAEDAATYYCQQWISNPLTFGAGTKLELK
7A2 HEAVY CHAIN (SEQ ID NO: 52)
MAVLVLFLCLAAFPSYVLSQVQMEQSGPGLVAPSQSLSITCTVSDFSLINYGVHWVRQSPGKG
LEWLGVIWTGGNTEYNSALMSRLSITKDNSKSQVFLKMNSLQADDTAMYYCARDDYYFGGGA
MDYWGQGTSVTVSS
7A2 LIGHT CHAIN (SEQ ID NO: 53)
MVSTPQFLVFLLFWIPASRGDILLTQSPAILSVSPGERVSFACRASQTIGTNIHWYQQRTNGSPR
LLIKYASESISEIPSRFSGSGSGTDFILTISSVESEDIADYYCQQSNNWPLTFGAGTKLELR
2F3 HEAVY CHAIN (SEQ ID NO: 54)
MEWRWIFLLLLSGTTGVHSEIQLQQSGPELVKPGASVKVSCKASGYTFTSFTLYWVKQSHGKS
LEWIGYIDPSNGGTTYNQRFKGKATLTVDKSSTTAYMHLNSLTSEDSSVYYCARGIYDGYYVG
KIFDYWGQGTTLTVSS
2F3 LIGHT CHAIN (SEQ ID NO:55)
METDTLLLWVLLLWVPGSTGNIVLTQSPTSLAVSLGQRATISCRASESVDNYGNSFMHWYQQK
PGQPPRLLIYLASNLESGVPARFSGSGSRTDFTLTIDPVEADDAATYYCQQNNEDPPTFGGGT
KLEIR
1B10 HEAVY CHAIN (SEQ ID NO: 56)
MGWSYIILFLVATATDVHSQVQLQQPGAELVKPGASVKLSCKASGYTFTSYWMDWVKQRPGQ
GLEWIGEIYPSNGRTKYNEKFKNKATLTVDNSSRTAYMHLSSLTSEDSAVYYCARGGYDVYYV
GNTLDYWGQGTSVTVSS
1B10 LIGHT CHAIN (SEQ ID NO: 57)
METDTLLLWVLLLWVPGSTGNIVLTQSPASLAVSLGQRATISCRASESIDSYGNSFMHWYHQK
PGQPPKLLIYLASNLESGVPARFSGSGSRTDFTLTIDPVEADDAATYYCLQNNGDPWTFGGGT
RLEIK
13C5 HEAVY CHAIN (SEQ ID NO: 58)
MECNWILPFILSVTSGVYSEVQLQQSGTVLARPGASVKMSCKASGYTFTSYWMHWVKQRPG
QGLEWIGAIYPGNSDTSYNQKFKGKAKLTAVTSTSTAYMELSSLTNEDSAVYYCTIYGNYGYFD
VWGAGTTVTVSS
13C5 LIGHT CHAIN (SEQ ID NO: 59)
MDSQAQVLMLLLLWVSGTCGDIVMSQSPSSLAVSVGEKVTMSCKSSQSLLYSSNQKNYLAWY
QQKPGQSPKLLIYWASTRESGVPDRFTGSGSGTDFTLTISSVKAEDLAVYYCQQYYSYPPYTF
GGGTKLEIK
15A3 HEAVY CHAIN (SEQ ID NO: 60)
MEWRIFLFILSGTACVHSQDQLLQSGPELVKPGTSVKMSCRASGYTFSDDVINWVRKRTGQGL
EWIGEIYPRIGSMYYNENFKGRATLTADKSSNTVYIHLSSLTSEDSAVYFCARYLLTKGYGMDY
WGQGTSVTVSS
15A3 LIGHT CHAIN (SEQ ID NO: 61)
METDTLLLWVLLLWVPGSTGDIVLTQSPASLAVSLGQRATISCRASKSVSPSDYSYIHWYQQKP
GQPPKLLIYLASNLESGVPARFSGSGSGTDFTLNIHPVEEEDAATYYCQHSRELPRTFGGGTKL
EIK
6A3 HEAVY CHAIN (SEQ ID NO: 62)
MAVLGLLFCLVTFPSCVLSQVQLKQSGPGLVQPSQSLSITCTVSGFSLTSYGLHWVRQSPGKG
LEWLGFIWSGGATDYNAAFISRLSISKDNSKRQVFFKMNSLQANDTAIYFCARRDDNYALAMDY
WGQGTSVTVSS
6A3 LIGHT CHAIN (SEQ ID NO: 63)
MDFQVQIFSFLLMSASVIMSRGQIVLTQSPALMSASPGEKVTMTCRASSSVSYIYWYQQKPRS
SPKPWIYLTSDLASGVPTRFSGSGSGTSYFLTISSMEAEDTATYYCQQWNSNPLTFGAGTKLE
LK
11C3 HEAVY CHAIN (SEQ ID NO: 64)
ATGCTGTTGGGGCTGAAGTGGGTTTTCTTTGTTGTTTTTTATCAAGGTGTGCATTGTGAGGT
AAAACTTGTTGAGTCTGGTGGAGGATTGGTGCAGCCTAAAGGGTCATTGAAACTCTCATGT
GCAGCCTCTGGATTCACCTTCAATACCTACGCCATGAACTGGATCCGCCAGGCTCCAGGA
AAGGGTTTGGAATGGGTTGCTCGCATAAGAAGTAAAAATAATTATTATGCAACATATTATGC
CGATTCAGTGAAAGACAGGTTCACCATCTCCAGAGATGATTCACAAAGTATGCTCTATCTG
CAAATGAACAACTTGAAAACTGAGGACACAGCCATGTATTACTGTGTGAGCTTTGCTTACTG
GGGCCAAGGGACTCTGGTCACTGTCTCTGCA
11C3 LIGHT CHAIN (SEQ ID NO: 65)
ATGAGGTTCTCTGCTCAGCTTCTGGGGCTGCTTGTGCTCTGGATCCCTGGATCCACTGCA
GAAATTGTGATGACGCAGGCTGCATTCTCCAATCCAGTCACTCTTGGAACATCAGCTTCCA
TCTCCTGCAGGTCTAGTAAGAGTCTCCTACATAGTAATGGCATCACTTATTTGTATTGGTAT
CTGCAGAAGCCAGGCCAGTCTCCTCGGTTCCTGATTTATCAGATGTCCAACCTTGCCTCAG
GAGTCCCAGACAGGTTCACTAGCAGTGGTTCAGGAACTGATTTCACACTGAGAATCAGCAG
AGTGGAGGCTGAGGATGTGGGTGTTTATTACTGTGCTCAAAATCTAGAACTTCCGTGGACG
TTCGGTGGAGGCACCAAGCTGGAAATCAAA
15D6 HEAVY CHAIN (SEQ ID NO: 66)
ATGGACTCCAGGCTCAATTTAGTTTTCCTTGTCCTTATTTTAAAAGGTGTCCAGTGTGATGT
GCAGCTGGTGGAGTCTGGGGGAGGCTTAGTGCAGCCTGGAGGGTCCCGGAAACTCTCCT
GTGCAGCCTCTGGATTCACTTTCAGCACCTTTGGAATGCACTGGGTTCGTCAGGCTCCAGA
GAAGGGGCTGGAGTGGGTCGCATACATCAGTAGTGGCAGTAGTTCCATCTACTATGCAGA
CACAGTGAAGGGCCGATTCACCATTTCCAGAGACAATCCCGTGAACACCCTGTTCCTGCAA
ATGACCAGTCTAAGGTCTGAAGACACGGCCATGTATTACTGTGCAAGATCCGGACTCGGTA
CTAGCCCCCATGCTATGGACTACTGGGGTCAAGGAACCTCAGTCACCGTCTCCTCA
15D6 LIGHT CHAIN (SEQ ID NO: 67)
ATGGATTTTCAAGTGCAGATTTTCAGCTTCCTGCTAATGAGTGCCTCAGTCATAATGTCCAG
GGGACAAATTGTTCTCACCCAGTCTCCAACACTCATGTCTGCATCTCCAGGGGAGAAGGTC
ACCATGACCTGCAGTGCCAGCTCAAGTGTAGGTTACATGTACTGGTACCAGCAGAAGCCA
AGATCCTCCCCCAAACCCTGGATTTATCTCACATCCATCCTGGCTTCTGGAGTCCCTGCTC
GCTTCAGTGGCAGTGGGTCTGGGACCTCTTACTCTCTCACAATCAGCAGCATGGAGGCTG
AAGATGCTGCCACTTATTACTGCCAGCAGTGGATTAGTAACCCGCTCACGTTCGGTGCTGG
GACCAAGCTGGAGCTGAAA
7A2 HEAVY CHAIN (SEQ ID NO: 68)
ATGGCTGTCCTGGTGCTGTTCCTCTGCCTGGCTGCATTTCCAAGCTATGTCCTGTCCCAGG
TGCAGATGGAGCAGTCAGGACCTGGCCTGGTGGCGCCCTCACAGAGCCTGTCCATCACTT
GCACTGTCTCTGACTTTTCACTAATTAACTATGGTGTTCACTGGGTTCGCCAGTCTCCAGGA
AAGGGTCTGGAGTGGCTGGGAGTCATTTGGACTGGTGGAAATACAGAATATAATTCGGCT
CTCATGTCCAGATTGAGCATCACCAAAGACAACTCCAAGAGCCAAGTTTTCTTAAAAATGAA
CAGTCTGCAAGCTGATGACACAGCCATGTACTACTGTGCCCGAGATGACTATTACTTCGGT
GGTGGGGCTATGGACTACTGGGGTCAAGGAACCTCAGTCACCGTCTCCTCA
7A2 LIGHT CHAIN (SEQ ID NO: 69)
ATGGTATCCACACCTCAGTTCCTTGTATTTTTGCTTTTCTGGATTCCAGCCTCCAGAGGCGA
CATCTTGTTGACTCAGTCTCCAGCCATCCTGTCTGTGAGTCCAGGAGAAAGAGTCAGTTTC
GCCTGCAGGGCCAGTCAGACCATTGGCACAAACATACACTGGTATCAACAACGAACAAAT
GGTTCTCCAAGGCTTCTCATAAAATATGCTTCTGAGTCTATCTCTGAGATCCCTTCCAGGTT
TAGTGGCAGTGGATCGGGGACAGATTTTATTCTCACCATCAGCAGTGTGGAGTCTGAAGAT
ATTGCAGATTATTACTGTCAACAAAGCAATAACTGGCCGCTCACGTTCGGTGCTGGGACCA
AGCTGGAGCTGAGA
2F3 HEAVY CHAIN (SEQ ID NO: 70)
ATGGAATGGAGATGGATCTTTCTCCTCCTCCTGTCAGGAACTACAGGTGTCCACTCTGAGA
TCCAGCTGCAGCAGTCTGGACCTGAGCTGGTGAAGCCTGGGGCTTCAGTGAAGGTTTCCT
GCAAGGCTTCTGGTTATACATTCACTAGCTTCACCTTGTACTGGGTGAAGCAGAGCCATGG
AAAGAGCCTTGAGTGGATTGGATATATTGATCCTTCCAATGGTGGTACTACCTACAACCAG
AGGTTCAAGGGCAAGGCCACATTGACTGTTGACAAGTCCTCCACCACAGCCTACATGCATC
TCAACAGCCTGACATCTGAGGACTCTTCAGTCTATTACTGTGCAAGAGGAATTTATGATGGT
TACTACGTAGGGAAAATTTTTGACTACTGGGGCCAGGGCACCACTCTCACAGTCTCCTCA
2F3 LIGHT CHAIN (SEQ ID NO: 71)
ATGGAGACAGACACACTCCTGCTATGGGTGCTGCTGCTCTGGGTTCCAGGTTCCACAGGT
AACATTGTACTGACCCAATCTCCAACTTCTTTGGCTGTGTCTCTCGGGCAGAGGGCCACCA
TATCCTGCAGAGCCAGTGAAAGTGTTGATAATTATGGCAATAGTTTTATGCACTGGTACCAG
CAGAAACCAGGACAGCCACCCAGACTCCTCATCTATCTTGCGTCCAACCTAGAATCTGGG
GTCCCTGCCAGGTTCAGTGGCAGTGGGTCTAGGACAGACTTCACCCTCACCATTGATCCT
GTGGAGGCTGATGATGCTGCAACCTATTACTGTCAGCAAAATAATGAGGATCCTCCGACGT
TCGGTGGAGGCACCAAGCTGGAAATCAGA
1B10 HEAVY CHAIN (SEQ ID NO: 72)
ATGGGATGGAGCTACATCATCCTCTTTTTGGTAGCAACAGCTACAGATGTCCACTCCCAGG
TCCAACTGCAGCAGCCTGGGGCTGAACTGGTGAAGCCTGGGGCTTCAGTGAAGCTGTCCT
GTAAGGCTTCTGGCTACACCTTCACCAGTTATTGGATGGACTGGGTGAAGCAGAGGCCTG
GACAAGGCCTTGAGTGGATTGGAGAGATTTATCCTAGCAACGGTCGTACTAAGTATAATGA
GAAGTTCAAGAACAAGGCCACACTGACTGTAGACAATTCCTCCAGGACAGCCTACATGCAT
CTAAGCAGCCTGACATCTGAGGACTCTGCGGTCTATTACTGTGCAAGAGGGGGCTATGAT
GTTTACTACGTCGGCAATACTTTGGACTACTGGGGTCAAGGAACCTCAGTCACCGTCTCCT
CA
1B10 LIGHT CHAIN (SEQ ID NO: 73)
ATGGAGACAGACACACTCCTGCTATGGGTGCTGCTGCTCTGGGTTCCAGGTTCCACAGGT
AACATTGTGTTGACCCAATCTCCAGCTTCTTTGGCTGTGTCTCTGGGGCAGAGGGCCACCA
TATCCTGCAGAGCCAGTGAAAGTATTGATAGTTATGGCAATAGTTTTATGCACTGGTACCAC
CAGAAACCAGGACAGCCACCCAAACTCCTCATTTATCTTGCATCCAACCTAGAATCTGGGG
TCCCTGCCAGGTTCAGTGGCAGTGGGTCTAGGACAGACTTCACCCTCACCATTGATCCTGT
GGAGGCTGATGATGCTGCCACCTATTACTGTCTCCAAAATAATGGGGATCCCTGGACGTTC
GGTGGAGGCACCCGGCTGGAGATCAAA
13C5 HEAVY CHAIN (SEQ ID NO: 74)
ATGGAATGTAACTGGATACTTCCTTTTATTCTGTCGGTAACTTCAGGGGTCTACTCAGAGGT
TCAGCTCCAGCAGTCTGGGACTGTGCTGGCAAGGCCTGGGGCTTCAGTGAAGATGTCCTG
CAAGGCTTCTGGCTACACCTTTACCAGCTACTGGATGCACTGGGTAAAACAGAGGCCTGG
ACAGGGTCTGGAATGGATTGGCGCTATTTATCCTGGAAATAGTGATACTAGCTACAACCAG
AAGTTCAAGGGCAAGGCCAAACTGACTGCAGTCACATCCACCAGCACTGCCTACATGGAG
CTCAGCAGCCTGACAAATGAGGACTCTGCGGTCTATTACTGTACAATCTATGGTAACTACG
GGTACTTCGATGTCTGGGGCGCAGGGACCACGGTCACCGTCTCCTCA
13C5 LIGHT CHAIN (SEQ ID NO: 75)
ATGGATTCACAGGCCCAGGTTCTTATGTTACTGCTGCTATGGGTATCTGGTACCTGTGGGG
ACATTGTGATGTCACAGTCTCCATCCTCCCTAGCTGTGTCAGTTGGAGAGAAGGTTACTAT
GAGCTGCAAGTCCAGTCAGAGCCTTTTATATAGTAGCAATCAAAAGAACTACTTGGCCTGG
TACCAGCAGAAACCAGGGCAGTCTCCTAAACTGCTGATTTACTGGGCATCCACTAGGGAAT
CTGGGGTCCCTGATCGCTTCACAGGCAGTGGATCTGGGACAGATTTCACTCTCACCATCA
GCAGTGTGAAGGCTGAAGACCTGGCAGTTTATTACTGTCAGCAATATTATAGCTATCCTCC
GTACACGTTCGGAGGGGGGACCAAGCTGGAAATAAAA
15A3 HEAVY CHAIN (SEQ ID NO:76)
ATGGAATGGAGGATCTTTCTCTTCATCCTGTCAGGAACTGCATGTGTCCACTCCCAGGATC
AGCTGCTGCAGTCTGGGCCTGAACTGGTGAAGCCTGGGACTTCAGTGAAGATGTCCTGCA
GGGCTTCTGGATACACATTCAGTGACGATGTCATAAACTGGGTGCGGAAGAGAACTGGAC
AGGGCCTGGAATGGATTGGAGAGATTTATCCTAGAATTGGTAGTATGTACTACAATGAGAA
TTTCAAGGGCAGGGCCACTCTGACTGCAGACAAATCGTCCAACACAGTCTACATTCATCTC
AGCAGCCTGACATCTGAGGACTCTGCGGTCTATTTCTGCGCACGATATCTACTAACGAAGG
GCTATGGTATGGACTACTGGGGTCAAGGCACCTCAGTCACCGTCTCCTCA
15A3 LIGHT CHAIN (SEQ ID NO: 77)
ATGGAGACAGACACACTCCTGTTATGGGTACTGCTGCTCTGGGTTCCAGGTTCCACTGGT
GACATTGTGCTGACACAGTCTCCTGCTTCCTTAGCTGTGTCTCTGGGGCAGAGGGCCACC
ATCTCATGCAGGGCCAGCAAAAGTGTCAGTCCATCTGACTATAGTTATATCCACTGGTACC
AACAGAAACCAGGACAGCCACCCAAACTCCTCATCTATCTTGCATCCAACCTAGAATCTGG
GGTCCCTGCCAGGTTCAGTGGCAGTGGGTCTGGGACAGACTTCACCCTCAACATCCATCC
TGTGGAGGAGGAGGATGCTGCAACCTATTACTGTCAACACAGTAGGGAGCTTCCTCGGAC
GTTCGGTGGAGGCACCAAGTTGGAAATCAAA
6A3 HEAVY CHAIN (SEQ ID NO: 78)
ATGGCTGTCTTGGGGCTGCTCTTCTGCCTGGTGACATTCCCAAGCTGTGTCCTATCCCAGG
TGCAGCTGAAACAGTCAGGACCTGGCCTAGTGCAGCCCTCACAGAGCCTGTCCATCACCT
GCACCGTCTCTGGTTTCTCATTAACTAGCTATGGTTTACACTGGGTTCGCCAGTCTCCAGG
AAAGGGTCTGGAGTGGTTGGGATTCATTTGGAGTGGTGGAGCCACAGACTATAATGCAGC
TTTCATATCCAGACTGAGCATCAGCAAGGACAATTCCAAGAGGCAAGTTTTCTTTAAAATGA
ACAGTCTGCAAGCTAATGACACAGCCATATATTTCTGTGCCAGAAGGGATGATAATTACGC
CTTGGCTATGGACTACTGGGGTCAAGGAACCTCAGTCACCGTCTCCTCA
6A3 LIGHT CHAIN (SEQ ID NO: 79)
ATGGATTTTCAAGTGCAGATTTTCAGCTTCCTGCTAATGAGTGCCTCAGTCATAATGTCCAG
GGGACAAATTGTTCTCACCCAGTCTCCAGCACTCATGTCTGCATCTCCAGGGGAGAAGGT
CACCATGACCTGCCGTGCCAGCTCAAGTGTAAGTTACATATACTGGTACCAGCAGAAGCCA
AGATCCTCCCCCAAACCCTGGATTTATCTCACATCCGACCTGGCTTCTGGAGTCCCTACTC
GCTTCAGTGGCAGTGGGTCTGGGACCTCTTACTTTCTTACAATCAGTAGCATGGAGGCTGA
AGATACTGCCACTTATTACTGCCAGCAGTGGAATAGTAATCCGCTCACATTCGGTGCTGGG
ACCAAGCTGGAACTGAAA
11C3 HEAVY CHAIN CDR 1 (SEQ ID NO: 80)
ACCTACGCCATGAAC
11C3 LIGHT CHAIN CDR 1 (SEQ ID NO: 81)
AGGTCTAGTAAGAGTCTCCTACATAGTAATGGCATCACTTATTTGTAT
11C3 HEAVY CHAIN CDR 2 (SEQ ID NO: 82)
CGCATAAGAAGTAAAAATAATTATTATGCAACATATTATGCCGATTCAGTGAAAGAC
11C3 LIGHT CHAIN CDR 2 (SEQ ID NO: 83)
CAGATGTCCAACCTTGCCTCA
11C3 HEAVY CHAIN CDR 3:
TTTGCTTAC
11C3 LIGHT CHAIN CDR 3 (SEQ ID NO: 84)
GCTCAAAATCTAGAACTTCCGTGGACG
15D6 HEAVY CHAIN CDR 1 (SEQ ID NO: 85)
ACCTTTGGAATGCAC
15D6 LIGHT CHAIN CDR 1 (SEQ ID NO: 86)
AGTGCCAGCTCAAGTGTAGGTTACATGTAC
15D6 HEAVY CHAIN CDR 2 (SEQ ID NO: 87)
TACATCAGTAGTGGCAGTAGTTCCATCTACTATGCAGACACAGTGAAGGGC
15D6 LIGHT CHAIN CDR 2 (SEQ ID NO: 88)
CTCACATCCATCCTGGCTTCT
15D6 HEAVY CHAIN CDR 3 (SEQ ID NO: 89)
TCCGGACTCGGTACTAGCCCCCATGCTATGGACTAC
15D6 LIGHT CHAIN CDR 3 (SEQ ID NO: 90)
CAGCAGTGGATTAGTAACCCGCTCACG
7A2 HEAVY CHAIN CDR 1 (SEQ ID NO: 91)
AACTATGGTGTTCAC
7A2 LIGHT CHAIN CDR 1 (SEQ ID NO: 92)
AGGGCCAGTCAGACCATTGGCACAAACATACAC
7A2 HEAVY CHAIN CDR 2 (SEQ ID NO: 93)
GTCATTTGGACTGGTGGAAATACAGAATATAATTCGGCTCTCATGTCC
7A2 LIGHT CHAIN CDR 2 (SEQ ID NO: 94)
TATGCTTCTGAGTCTATCTCT
7A2 HEAVY CHAIN CDR 3 (SEQ ID NO: 95)
GATGACTATTACTTCGGTGGTGGGGCTATGGACTAC
7A2 LIGHT CHAIN CDR 3 (SEQ ID NO: 96)
CAACAAAGCAATAACTGGCCGCTCACG
2F3 HEAVY CHAIN CDR 1 (SEQ ID NO: 97)
AGCTTCACCTTGTAC
2F3 LIGHT CHAIN CDR 1 (SEQ ID NO: 98)
AGAGCCAGTGAAAGTGTTGATAATTATGGCAATAGTTTTATGCAC
2F3 HEAVY CHAIN CDR 2 (SEQ ID NO: 99)
TATATTGATCCTTCCAATGGTGGTACTACCTACAACCAGAGGTTCAAGGGC
2F3 LIGHT CHAIN CDR 2 (SEQ ID NO: 100)
CTTGCGTCCAACCTAGAATCT
2F3 HEAVY CHAIN CDR 3 (SEQ ID NO: 101)
GGAATTTATGATGGTTACTACGTAGGGAAAATTTTTGACTAC
2F3 LIGHT CHAIN CDR 3 (SEQ ID NO: 102)
CAGCAAAATAATGAGGATCCTCCGACG
1B10 HEAVY CHAIN CDR 1 (SEQ ID NO: 103)
AGTTATTGGATGGAC
1B10 LIGHT CHAIN CDR 1 (SEQ ID NO: 104)
AGAGCCAGTGAAAGTATTGATAGTTATGGCAATAGTTTTATGCAC
1B10 HEAVY CHAIN CDR 2 (SEQ ID NO: 105)
GAGATTTATCCTAGCAACGGTCGTACTAAGTATAATGAGAAGTTCAAGAAC
1B10 LIGHT CHAIN CDR 2 (SEQ ID NO: 106)
CTTGCATCCAACCTAGAATCT
1B10 HEAVY CHAIN CDR 3 (SEQ ID NO: 107)
GGGGGCTATGATGTTTACTACGTCGGCAATACTTTGGACTAC
1B10 LIGHT CHAIN CDR 3 (SEQ ID NO: 108)
CTCCAAAATAATGGGGATCCCTGGACG
13C5 HEAVY CHAIN CDR 1 (SEQ ID NO: 109)
AGCTACTGGATGCAC
13C5 LIGHT CHAIN CDR 1 (SEQ ID NO: 110)
AAGTCCAGTCAGAGCCTTTTATATAGTAGCAATCAAAAGAACTACTTGGCC
13C5 HEAVY CHAIN CDR 2 (SEQ ID NO: 111)
GCTATTTATCCTGGAAATAGTGATACTAGCTACAACCAGAAGTTCAAGGGC
13C5 LIGHT CHAIN CDR 2 (SEQ ID NO: 112)
TGGGCATCCACTAGGGAATCT
13C5 HEAVY CHAIN CDR 3 (SEQ ID NO: 113)
TATGGTAACTACGGGTACTTCGATGTC
13C5 LIGHT CHAIN CDR 3 (SEQ ID NO: 114)
CAGCAATATTATAGCTATCCTCCGTACACG
15A3 HEAVY CHAIN CDR 1 (SEQ ID NO: 115)
GACGATGTCATAAAC
15A3 LIGHT CHAIN CDR 1 (SEQ ID NO: 116)
AGGGCCAGCAAAAGTGTCAGTCCATCTGACTATAGTTATATCCAC
15A3 HEAVY CHAIN CDR 2 (SEQ ID NO: 117)
GAGATTTATCCTAGAATTGGTAGTATGTACTACAATGAGAATTTCAAGGGC
15A3 LIGHT CHAIN CDR 2 (SEQ ID NO: 118)
CTTGCATCCAACCTAGAATCT
15A3 HEAVY CHAIN CDR 3 (SEQ ID NO: 119)
TATCTACTAACGAAGGGCTATGGTATGGACTAC
15A3 LIGHT CHAIN CDR 3 (SEQ ID NO: 120)
CAACACAGTAGGGAGCTTCCTCGGACG
6A3 HEAVY CHAIN CDR 1 (SEQ ID NO: 121)
AGCTATGGTTTACAC
6A3 LIGHT CHAIN CDR 1 (SEQ ID NO: 122)
CGTGCCAGCTCAAGTGTAAGTTACATATAC
6A3 HEAVY CHAIN CDR 2 (SEQ ID NO: 123)
TTCATTTGGAGTGGTGGAGCCACAGACTATAATGCAGCTTTCATATCC
6A3 LIGHT CHAIN CDR 2 (SEQ ID NO: 124)
CTCACATCCGACCTGGCTTCT
6A3 HEAVY CHAIN CDR 3 (SEQ ID NO: 125)
AGGGATGATAATTACGCCTTGGCTATGGACTAC
6A3 LIGHT CHAIN CDR 3 (SEQ ID NO: 126)
CAGCAGTGGAATAGTAATCCGCTCACA
Linker sequence:
GGGGSGGGGGGGGS (SEQ ID NO: 127)
Polypeptide sequence for 7A2 scFv (no secretion signal)(SEQ ID NO: 128):
QVQMEQSGPGLVAPSQSLSITCTVSDFSLINYGVHWVRQSPGKGLEWLGVIWTGGNTEYNSA
LMSRLSITKDNSKSQVFLKMNSLQADDTAMYYCARDDYYFGGGAMDYWGQGTSVTVSSGGG
GSGGGGSGGGGSDILLTQSPAILSVSPGERVSFACRASQTIGTNIHWYQQRTNGSPRLLIKYAS
ESISEIPSRFSGSGSGTDFILTISSVESEDIADYYCQQSNNWPLTFGAGTKLELRAAAYPYDVPD
YAHHHHHH
Polypeptide sequence for 7A2 scFv (Secretion signal included)(SEQ ID NO: 129)
ATMETDTLLLWVLLLWPGSTGQVQMEQSGPGLVAPSQSLSITCTVSDFSLINYGVHWVRQSP
GKGLEWLGVIWTGGNTEYNSALMSRLSITKDNSKSQVFLKMNSLQADDTAMYYCARDDYYFG
GGAMDYWGQGTSVTVSSGGGGSGGGGSGGGGSDILLTQSPAILSVSPGERVSFACRASQTI
GTNIHWYQQRTNGSPRLLIKYASESISEIPSRFSGSGSGTDFILTISSVESEDIADYYCQQSNNW
PLTFGAGTKLELRAAAYPYDVPDYAHHHHHH
Secretion sequence: ATMETDTLLLWVLLLWVPGSTG (SEQ ID NO: 130)
Polypeptide sequence for 7A2-IL22 (no secretion signal)(SEQ ID NO: 131)
QVQMEQSGPGLVAPSQSLSITCTVSDFSLINYGVHWVRQSPGKGLEWLGVIWTGGNTEYNSA
LMSRLSITKDNSKSQVFLKMNSLQADDTAMYYCARDDYYFGGGAMDYWGQGTSVTVSSGGG
GSGGGGSGGGGSDILLTQSPAILSVSPGERVSFACRASQTIGTNIHWYQQRTNGSPRLLIKYAS
ESISEIPSRFSGSGSGTDFILTISSVESEDIADYYCQQSNNWPLTFGAGTKLELRGGGGSGGGG
SGGGGSGGGGSGGGGSAPISSHCRLDKSNFQQPYITNRTFMLAKEASLADNNTDVRLIGEKLF
HGVSMSERCYLMKQVLNFTLEEVLFPQSDRFQPYMQEVVPFLARLSNRLSTCHIEGDDLHIQR
NVQKLKDTVKKLGESGEIKAIGELDLLFMSLRNACIAAAYPYDVPDYAHHHHHH
polypeptide sequence for 7A2-IL22 (SEQ ID NO: 132)
ATMETDTLLLWVLLLWPGSTGQVQMEQSGPGLVAPSQSLSITCTVSDFSLINYGVHWVRQSP
GKGLEWLGVIWTGGNTEYNSALMSRLSITKDNSKSQVFLKMNSLQADDTAMYYCARDDYYFG
GGAMDYWGQGTSVTVSSGGGGSGGGGSGGGGSDILLTQSPAILSVSPGERVSFACRASQTI
GTNIHWYQQRTNGSPRLLIKYASESISEIPSRFSGSGSGTDFILTISSVESEDIADYYCQQSNNW
PLTFGAGTKLELRGGGGSGGGGSGGGGSGGGGSGGGGSAPISSHCRLDKSNFQQPYITNRT
FMLAKEASLADNNTDVRLIGEKLFHGVSMSERCYLMKQVLNFTLEEVLFPQSDRFQPYMQEVV
PFLARLSNRLSTCHIEGDDLHIQRNVQKLKDTVKKLGESGEIKAIGELDLLFMSLRNACIAAAYPY
DVPDYAHHHHHH
Amino acid sequence for optional Flag tag (SEQ ID NO: 133):
DYKDDDDK
DNA sequence for 7A2-IL22 (SEQ ID NO: 134)
GCCACCATGGAAACCGACACACTGCTGCTGTGGGTGCTGCTTCTTTGGGTGCCCGGATCT
ACAGGCCAGGTGCAGATGGAACAGTCTGGCCCTGGACTGGTGGCCCCATCTCAGAGCCT
GAGCATCACCTGTACCGTGTCCGACTTCAGCCTGATCAACTACGGCGTGCACTGGGTCCG
ACAGAGCCCTGGAAAAGGACTGGAATGGCTGGGCGTGATCTGGACCGGCGGCAACACAG
AGTACAACAGCGCCCTGATGAGCCGGCTGTCCATCACCAAGGACAACAGCAAGAGCCAGG
TGTTCCTGAAGATGAACTCCCTGCAGGCCGACGACACCGCCATGTACTACTGCGCCAGAG
ATGACTACTACTTCGGCGGAGGCGCCATGGATTATTGGGGCCAGGGAACAAGCGTGACCG
TGTCTAGCGGAGGCGGAGGATCTGGTGGCGGAGGTAGTGGTGGCGGCGGATCTGATATT
CTGCTGACACAGTCCCCTGCCATCCTGTCCGTTTCTCCAGGCGAGAGAGTGTCCTTCGCC
TGTAGAGCCTCTCAGACCATCGGCACCAACATCCACTGGTATCAGCAGCGGACCAACGGC
AGCCCCAGACTGCTGATTAAGTACGCCAGCGAGAGCATCAGCGAGATCCCCAGCAGATTT
TCTGGCAGCGGCTCCGGCACCGATTTCATCCTGACAATCAGCTCCGTGGAAAGCGAGGAT
ATCGCCGATTACTACTGCCAGCAGAGCAACAACTGGCCCCTGACATTTGGAGCCGGCACC
AAGCTGGAACTTAGAGGCGGCGGAGGTTCTGGCGGTGGTGGATCTGGCGGAGGTGGAAG
CGGCGGAGGCGGCTCTGGCGGCGGAGGAAGTGCTCCTATTAGCAGCCACTGCCGGCTG
GACAAGAGCAACTTCCAGCAGCCTTACATCACCAACCGGACCTTCATGCTGGCCAAAGAG
GCCAGCCTGGCCGACAACAATACTGACGTGCGGCTGATCGGCGAGAAGCTGTTTCATGGC
GTGTCCATGAGCGAGCGGTGCTACCTGATGAAGCAGGTCCTGAACTTCACCCTGGAAGAG
GTGCTGTTCCCTCAGAGCGACCGGTTTCAGCCCTACATGCAAGAGGTGGTGCCCTTTCTG
GCCCGGCTGAGCAATAGACTGAGCACCTGTCACATCGAGGGCGACGACCTGCACATCCA
GAGAAACGTGCAGAAACTGAAGGACACCGTGAAGAAGCTGGGCGAGAGCGGAGAGATCA
AGGCCATCGGAGAACTGGACCTGCTGTTCATGAGCCTGCGGAACGCCTGTATCGCCGCTG
CCTATCCTTACGACGTGCCCGATTATGCCCACCACCACCATCACCACTGATGATGA
DNA Sequence for 7A2 scFv (SEQ ID NO: 135)
GCCACCATGGAAACCGACACACTGCTGCTGTGGGTGCTGCTTCTTTGGGTGCCCGGATCT
ACAGGCCAGGTGCAGATGGAACAGTCTGGCCCTGGACTGGTGGCCCCATCTCAGAGCCT
GAGCATCACCTGTACCGTGTCCGACTTCAGCCTGATCAACTACGGCGTGCACTGGGTCCG
ACAGAGCCCTGGAAAAGGACTGGAATGGCTGGGCGTGATCTGGACCGGCGGCAACACAG
AGTACAACAGCGCCCTGATGAGCCGGCTGTCCATCACCAAGGACAACAGCAAGAGCCAGG
TGTTCCTGAAGATGAACTCCCTGCAGGCCGACGACACCGCCATGTACTACTGCGCCAGAG
ATGACTACTACTTCGGCGGAGGCGCCATGGATTATTGGGGCCAGGGAACAAGCGTGACCG
TGTCTAGCGGAGGCGGAGGATCTGGTGGCGGAGGTAGTGGTGGCGGCGGATCTGATATT
CTGCTGACACAGTCCCCTGCCATCCTGTCCGTTTCTCCAGGCGAGAGAGTGTCCTTCGCC
TGTAGAGCCTCTCAGACCATCGGCACCAACATCCACTGGTATCAGCAGCGGACCAACGGC
AGCCCCAGACTGCTGATTAAGTACGCCAGCGAGAGCATCAGCGAGATCCCCAGCAGATTT
TCTGGCAGCGGCTCCGGCACCGATTTCATCCTGACAATCAGCTCCGTGGAAAGCGAGGAT
ATCGCCGATTACTACTGCCAGCAGAGCAACAACTGGCCCCTGACATTTGGAGCCGGCACC
AAGCTGGAACTGAGAGCCGCCGCTTATCCCTACGACGTGCCAGACTATGCCCACCACCAC
CATCACCACTGATGATGA

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the constructs of the present invention and practice the claimed methods. The following working examples therefore, specifically point out the typical aspects of the present invention and are not to be construed as limiting in any way in the remainder of the disclosure. Although specific terms have been employed herein, such terms are intended in a descriptive sense and not for purposes of limitation.

### Examples

Statistical analysis used for the Examples:

Data were analyzed using GraphPad Prism 8.0 (GraphPad Software, La Jolla, CA, U.S.A.). The statistical significance between 2 groups (or more) was determined using a student t-test or a one-way ANOVA with multiple-comparison test. P values of less than 0.05 were considered statistically significant.

Materials and Methods used for the Examples:

### Monoclonal Antibody Generation

A protein encompassing the 2 extracellular Ig domains of human MAdCAM-1 (Val23-Glu231) as well as the full length extracellular domain of hMAdCAM-1 were cloned into pcDNA 3.4 TOPO vectors (Thermofisher Gene Art Gene Synthesis) and expressed in Expi293F cells. Each protein construct harbored a C-terminus HIS tag and were purified using a His Trap Excel column (Cytiva). Mice (C57BL/6J and Balb/c) were vaccinated with the truncated MAdCAM-1 (lg domains) protein and hybridomas-producing mAbs against human MAdCAM-1 were generated by GenScript (New Jersey, USA). The hybridomas were maintained in Dulbecco's modified Eagle medium (DMEM) (WISENT) supplemented with 20% HyClone FetalClone II Serum (Cytiva), 1% penicillin-streptomycin (P/S) (WISENT), 1× HT Supplement (Gibco), 1× 2-mercaptoethanol (MilliporeSigma), and 25 mM HEPES (Gibco). For mAb production purposes, the hybridomas were adapted to grow in H-CELL serum-free medium (WISENT). The mAbs were purified from culture supernatants using HiTrap protein G HP columns (Cytiva). Bound mAbs were eluted from the column using 0.1 M glycine-HCl (pH 2.7) and neutralized with 1 M Tris-HCl (pH 9.0). Recovered mAbs were exchanged into phosphate buffered saline (PBS) using PD-10 desalting columns (Cytiva) and used immediately or stored at −80° C. The purity of the mAbs was confirmed by SDS-PAGE and Western blot using an anti-mouse IgG h+I Ab conjugated to horseradish peroxidase (HRP) (Bethyl Laboratories). Note, all other characterization of the anti-human mAbs were completed with the full length extracellular domain protein.

### Monoclonal Antibody Sequencing

The variable region of the heavy (VH) and light (VL) regions of eight hybridoma clones were sequenced by GenScript (New Jersey, USA). The clones were phylogenetically grouped based on sequence similarity using Clustal Omega (www.ebi.ac.uk/Tools/msa/clustalo/; EMBL-EBI, Hinxton, Cambridgeshire, UK) 305

### Reference Cited

305. Sievers, F. et al. Fast, scalable generation of high-quality protein multiple sequence alignments using Clustal Omega. Mol Syst Biol 7, (2011).

### Surface Plasmon Resonance

The binding kinetics of the mAbs to hMAdCAM-1 were determined by surface plasmon resonance using a BIAcore T200 (Cytiva) and HBS running buffer (20 mM of HEPES pH 7.4, 150 mM NaCl, 0.005% Tween-20, 3.4 mM EDTA). Briefly, the mAbs were immobilized on a protein G sensor chip (Cytiva). Sensograms were generated using single-cycle kinetics covering 5 concentrations (1:2 serial dilution) of hMAdCAM injected at a flow rate of 30 μL/min for 300s. RU values were recorded for 180s followed by a regeneration step. The sensorgrams were analyzed with a 1:1 Langmuir binding model after the reference sensorgrams were subtracted, to determine the KD, kₒₙ, and k_{off}.

### Western Blot Antigen Detection

The binding of hMAdCAM1 mAbs to the full length MAdCAM1 protein was also validated by Western Blot whereby 1 μg of hMAdCAM was loaded onto NuPage 4-12%, Bis-Tris gels (Invitrogen) and run at 200 V for 35 minutes. Gels were transferred (48 mM Tris base, 39 mM glycine, 20% v/v methanol) to Amersham™Protran™ 0.45-μm nitrocellulose blotting membrane (Cytiva). Membranes were blocked overnight in 5% milk at 4° C. One μg of each antibody was added and incubated at room temperature for 1 hr. The membranes were washed with Tris Buffer Saline (TBS)+0.1% Tween 20 prior to the addition of the secondary antibody (anti-mouse IgG h+I Ab conjugated to HRP; Bethyl Laboratories). Membranes were washed again before staining with Luminata™ Crescendo (Millipore Sigma) and imaged on a Fusion FX (Vilber) chemiluminescent imager with a 30-second exposure.

### Direct Cell Binding

Expi293F cells were transiently transfected with pcDNA 3.4 TOPO vector containing the complete gene for human MAdCAM-1 (Q13477) (Thermofisher Gene Art Gene Synthesis). Cells were harvested 24 hours post-transfection, washed with PBS, and incubated with 1 μg of each mAb, or a mouse IgG₁ (Biolegend), or a positive anti-hMAdCAM-1 control antibody (clone 17F5; Invitrogen). After 30 minutes at 4° C., the cells were incubated with a secondary antibody (0.5 μL anti-mouse IgG-FITC; Biolegend) for 30 minutes at 4° C. Cells were washed in 2 ml of PBS and resuspended in 3 μM DAPI (Thermo Fisher Scientific) followed by fluorescence-activated cell sorting (FACS) analysis using a BD LSR II flow cytometer (BD) maintained by The Center for Flow Cytometry & Scanning Microscopy (CCSM) at Sunnybrook Research Institute (SRI; Sunnybrook Health Sciences Center, Toronto, ON, Canada).

### Human Intestinal Tissue Histology

Paraffin-embedded healthy adult male small intestine tissue sections were purchased from BioChain Institute, Inc. Following deparaffinization, the slides were incubated with 3% hydrogen peroxide (in methanol) for 15 min at room temperature (RT) to block endogenous peroxidase activity and subsequently treated with anti-goat serum (Biolegend) followed by avidin/biotin blocking reagents (Biolegend). The samples were then incubated for 1 h at RT with each human anti-MAdCAM-1 mAb (as well as the biotinylated anti-hMAdCAM-1 7A2scFv-IL22 bispecific) diluted at 10 μg/ml (TBST; TBS+0.1% Tween). Anti-hMAdCAM-1 mAb clone 314G8 (Bio-Rad) was used as a positive control mAb. For detection, a secondary antibody (biotinylated goat anti-mouse IgG; Biolegend) (Note: no secondary was applied to the 7A2scFv-IL22 construct) was added for 1 h at RT followed by streptavidin-HRP (Biolegend). Slide washing cycles using TBST were performed after completing each step. The signal was developed using a DAB substrate kit (Vector Laboratories) in accordance with the manufacturer instructions. The samples were then counterstained with Mayer's Modified Hematoxylin solution (abcam), dehydrated, and mounted for microscopy analysis.

### Generation of MAdCAM-1 Targeting Bispecifics

Human MAdCAM-1 targeting bispecifics were generated by fusing a scFv derived from the human MAdCAM-1 mAb 7A2 (this study) to human IL22 (Q9GZX6-1). All genes were cloned into the pcDNA3.4 TOPO mammalian expression plasmid (GeneArt; Thermo Fisher Scientific) and expressed using the Expi293F protein expression system (Thermo Fisher Scientific). A 5′ lg-K leader sequence was included in each construct for high protein secretion by mammalian cells. Secreted proteins were subsequently purified by affinity chromatography using a HisTrap Excel column (Cytiva). Proteins were eluted with 0.5M imidazole and desalted into PBS pH 7.4 using PD-10 columns (Cytiva). Protein purity was assessed using SDS page and Western Blot using anti-HIS (anti-poly Histidine-mAb [clone His-1] peroxidase conjugate; Millipore Sigma) detection

### REFERENCES CITED

306. Streeter, P. R., Berg, E. L., Rouse, B. T. N., Bargatze, R. F. & Butcher, E. C. A tissue-specific endothelial cell molecule involved in lymphocyte homing. Nature 331, 41-46 (1988).

### Sandwich ELISA

A sandwich ELISA was performed to determine if human anti MAdCAM-1 7A2scFv-IL22 can simultaneously bind human IL22Rα and human MAdCAM-1. Human IL22Rα (2 μg/ml; R&D Systems) was dispensed into wells of a high-bind ELISA plate (Corning). The plates were blocked with 1% BSA at room temperature for 1 hr. M89scFv-IL22 (4 μg/ml) or molarity-matched MECA89 scFv alone was then added to wells and the plate incubated for one hour at room temperature. Recombinant murine MAdCAM-1.Fc (4 μg/ml; R&D Systems) was similarly added to each well. Finally, anti-human IgG-HRP conjugate was added and incubated for 40 minutes at room temperature. The plate was washed 3 times between each step with TBST (TBS+0.1% Tween). The substrate TMB (Molecular Innovations) was dispensed into each well and the color was read at 450 nm (after xx minutes) using a microplate reader (Synergy H1). Average optical density measurements+SD were derived from experiments performed in triplicate.

### Protein Modelling

A predicted ribbon structure of 7A2scFv-IL22 was generated using I-Tasser software³⁰⁸-³¹¹. The model with the best confidence score (C-score) is presented in this study.

### REFERENCES CITED

308. Roy, A., Kucukural, A. & Zhang, Y. I-TASSER: a unified platform for automated protein structure and function prediction. Nat Protoc 5, 725-738 (2010).
309. Yang, J. & Zhang, Y. I-TASSER server: New development for protein structure and function predictions. Nucleic Acids Res 43, W174-W181 (2015).
310. Zhang, Y. I-TASSER server for protein 3D structure prediction. BMC Bioinformatics 9, (2008).
311. The Yang Zhang Lab. https://zhanggroup.org/.

### In Vitro IL10 Secretion Assay

COLO205 cells were generously provided by Dr. Rima Al-awar. The cells were maintained in RPMI-1640 supplemented with 10% FCS, 100 U/ml penicillin, and 100 μg/ml of streptomycin. COLO205 cells are expected to release IL10 upon IL22 stimulation³¹². For the IL10 secretion assay, 3×10⁵ COLO205 cells were plated in wells of a 24-well plate tissue culture plate. The cells were stimulated with 1 ng/ml of recombinant mouse IL22, 7A2 mAb, or 7A2scFv-IL22 and incubated at 37° C. for 24 hours. The supernatant from each well was collected and assayed for the presence of IL10 using a human IL-10 cytokine ELISA kit (Biolegend) according to the manufacturer's instructions.

### Reference Cited

312. Nagalakshmi, M. L., Rascle, A., Zurawski, S., Menon, S. & De Waal Malefyt, R. Interleukin-22 activates STAT3 and induces IL-10 by colon epithelial cells. Int Immunopharmacol 4, 679-691 (2004).

### STAT3 Phosphorylation

COLO205 cells were cultured and stimulated with the IL22 constructs as described above. Following a 20-minute incubation period, the cells were harvested, and lysed using RIPA buffer (25 mM Tris-HCl, pH 7.6, 150 mM NaCl, 1% NP-40, 1% sodium deoxycholate, 0.1% SDS)+ protease and phosphatase inhibitor cocktail (ThermoFisher) by rotating the samples for 20 minutes at 4° C. Cellular lysates were cleared using centrifugation (12,000 rpm for 15 minutes). Samples were run on a 10-12% SDS PAGE (Invitrogen) and transferred to a 0.45 μm pore size polyvinylidene difluoride (PVDF) membrane (Cytiva). Following transfer, the membranes were blocked in a 3% Bovine Serum Albumin (BioShop) solution in TBST (10 mM Tris-HCl, pH 8.0, 0.150 mM NaCl, 0.1% Tween 20) overnight at 4° C. The membranes were cut and subsequently incubated with a 1:1000 dilution of anti-Phospho-STAT3 (Tyr705, ThermoFisher) antibody and anti-BActin (ThermoFisher) for 1 hr at room temperature. Membranes were washed in TBST and incubated with a 1:10,000 dilution of anti-rabbit HRP-conjugated secondary antibody (Bethyl) at room temperature for 1 h. HRP activity was detected with Luminata™ Crescendo (Millipore Sigma) and imaged on a Fusion FX (Vilber) chemiluminescent imager.

### Example 1 Generation and Binding Characterization of Anti-Human MAdCAM-1 Antibodies

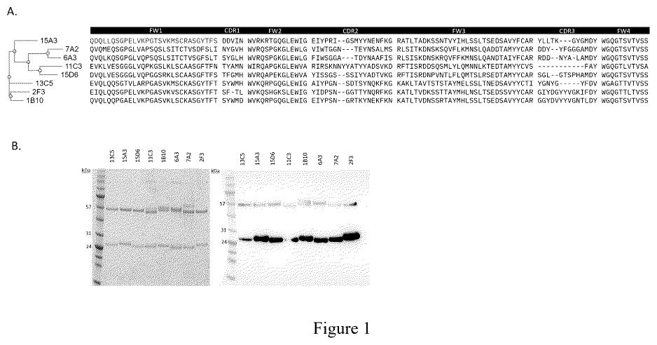

Anti-human MAdCAM-1 mAbs were initially generated in order to construct human MAdCAM-1 scFvs and related targeted bispecifics. A truncated MAdCAM-1 protein representing the two N-terminal Ig-like domains of hMAdCAM-1 served as the antigen. Eight hybridoma clones were selected, based on ELISA screens against hMAdCAM-1 with all generated clones expressing IgG₁ isotype murine mAbs. The CDR3 region is typically associated with antigen specificity³⁸. Accordingly, the heavy and light variable regions of the eight clones were sequenced and grouped into 5 different families based on sequence similarity in the heavy chain (FIG. 1A). We confirmed the purity of the mAbs by SDS-PAGE and Western blot detected with an anti-mouse IgG h+I chain antibody (FIG. 1B).

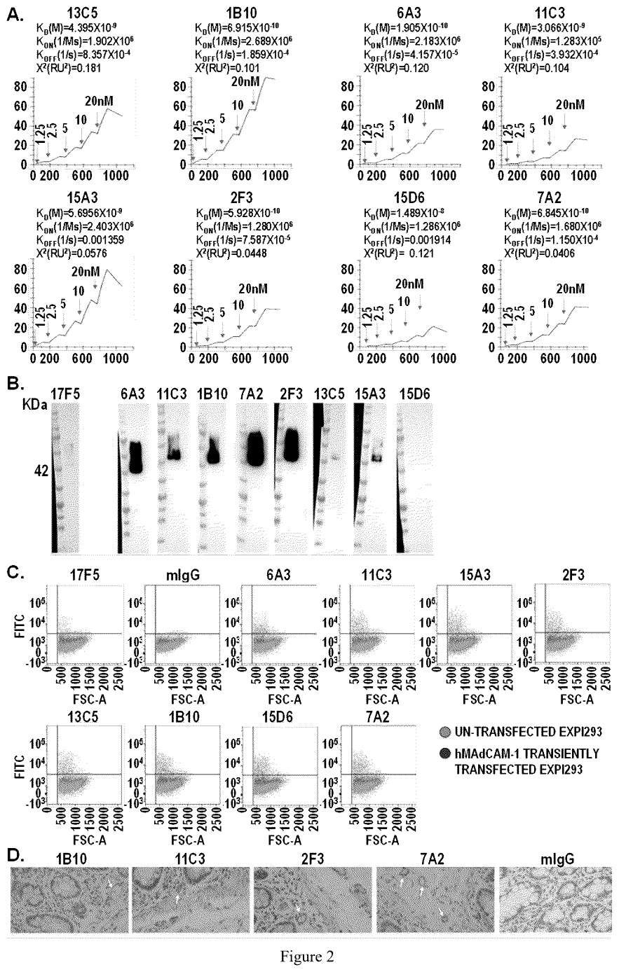

Antibodies derived from all clones bound tightly to recombinant human MAdCAM-1 (hMAdCAM-1) with Kps in the low nM range (0.19 nM to 14 nM) as calculated from surface plasmon resonance (SPR) measurements (FIG. 2A). As expected, we did not observe any binding toward mouse MAdCAM-1 (data not shown). Furthermore, we assayed whether the mAbs could bind hMAdCAM-1 by Western blot. Antibodies generated by all clones tested, except for 15D6, were able to detect the hMAdCAM-1 band transferred to a membrane (1 μg of each antibody; 30-second exposure time) (FIG. 2B). The ability of these mAbs to recognize hMAdCAM-1 expressed was also confirmed in a more clinically-relevant context such as on the surface of cells and on intact human intestinal tissue sections. Briefly, HEK cells (Expi293F) were transiently transfected to express human MAdCAM-1 on their surface. The expression of hMAdCAM was confirmed using a commercially available positive control mAb (clone 17F5; Invitrogen). As shown in FIG. 2C, all the clones tested except the IgG isotype control were able to detect hMAdCAM-1 expression on transfected cells, but not the non-transfected cells. More importantly, immunohistochemistry performed on intact human small intestine sections revealed that mAbs from clones 7A2, 2F3, 1B10, and 11C3 detected human MAdCAM-1 expression on intact human small intestine via histology (FIG. 2D). Based on these binding results, production yields, and irrespective of potential competitive disruption of a437 integrin binding, clone 7A2 was selected to further develop human MAdCAM-1 targeting scFv and related bispecifics.

### Reference Cited

38. Xu, J. L. & Davis, M. M. Diversity in the CDR3 region of V(H) is sufficient for most antibody specificities. Immunity 13, 37-45 (2000).

### Example 2 T Cell Co-Stimulation is Inhibited by Anti-MAdCAM-1 Antibodies

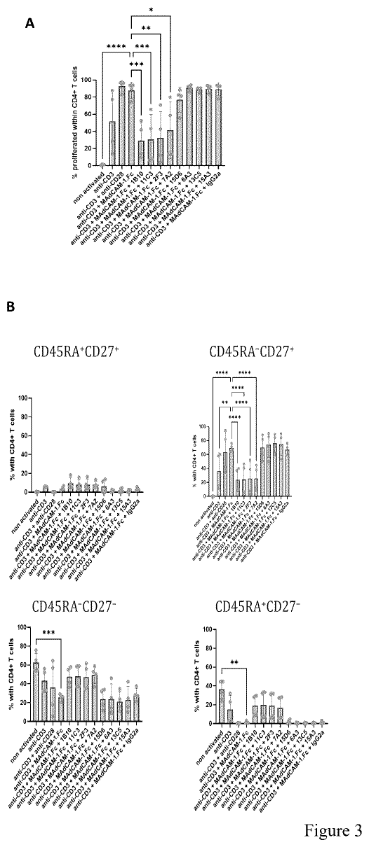

To determine whether any of the mAb clones could block the MAdCAM-1/a4B7 integrin interaction, we performed a MAdCAM-1 T cell co-stimulation assay. Herein, CFSE-labelled T cells isolated from the blood of healthy donors (n=4) were stimulated with a suboptimal concentration of plate-bound anti-CD3 co-coated with MAdCAM-1 in the presence or absence of the mAbs produced in Example 1. Following 4 days in culture, we assessed the impact of the mAbs on the proliferation and phenotype of the CD4+ T cells. As shown in FIG. 3A, MAdCAM-1 co-stimulation caused a significant increase in the proliferation of CD4+ T cells relative to unstimulated cells and was greatly enhanced compared to anti-CD3 alone. The addition of clones 1B10, 11C3, 2F3, and 7A2 but not 15D6, 6A3, 13C5, 15A3, or the irrelevant IgG control, blocked MAdCAM-1 co-stimulation evidenced by a significant reduction in the proliferation of the CD4+ T cells. We further corroborated these results by examining evidence of phenotypic changes in the expanding T cell population. As shown in FIG. 3B, MAdCAM-1 co-stimulation preferentially expanded the CD45RA⁻CD27+ central memory like CD4+ T cells. Upon adding the mAbs, 1B10, 11C3, 2F3, and 7A2 there was a significant reduction in the % of CD45RA⁻CD27+ cells within the CD4+ T cell population, rather favoring the expansion of CD45RA⁻CD27⁻ similar to that observed for anti-CD3 stimulation alone. These results indicate that 4 clones, 1B10, 11C3, 2F3, and 7A2, may be considered MAdCAM-1 blocking antibodies.

### Example 3 Design and Production of Human MAdCAM-1 Targeting Bispecific 7A2scFv-IL22

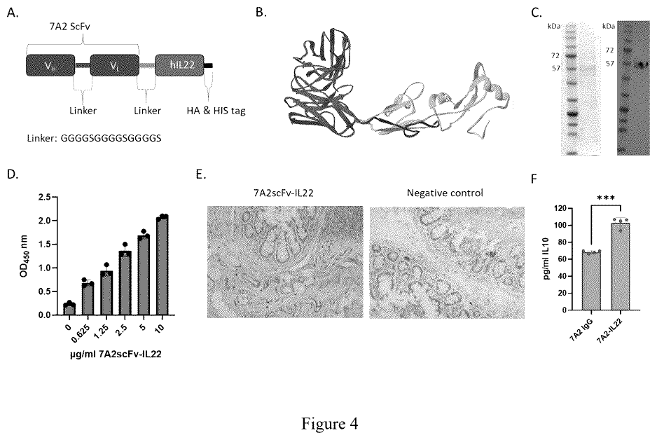

A hMAdCAM-1 targeting bispecific was designed by fusing a scFv derived from 7A2 to human IL22 (FIG. 4A). The domains were linearly linked together using the flexible linker GGGGSGGGGSGGGGS (SEQ ID NO:129) 66. HA and HIS tags were added to the C-terminus of the constructs to facilitate their purification and detection. The predicted ribbon structure for the anti hMAdCAM-1 7A2scFv-IL22 bispecific, as determined using I-TASSER (confidence score or C-Score of −0.96), is shown in FIG. 4B). The construct was expressed as a secreted soluble protein in EXPI293F cells and purified by affinity chromatography. SDS-PAGE and Western blot confirmed the purity of the construct. As shown in FIG. 4C, the SDS-PAGE band corresponding to 7A2scFv-IL22 migrated with a slightly higher apparent mass or ˜57 kDa than expected from its non-glycosylated molecular weight of 46 kDa. A bridging ELISA was also performed to confirm that hMAdCAM-1 7A2scFv-IL22 did bind to both IL22 and MAdCAM-1 simultaneously (FIG. 4D). In this assay, ELISA plate wells were initially coated with IL22Rα. Increasing concentrations of hMAdCAM-1 7A2scFv-IL22 bispecific were then dispensed in the wells and the resulting bound trimolecular complex with biotinylated recombinant hMAdCAM-1 was then detected using streptavidin-HRP. The dose-dependent increase in signal demonstrates that the hMAdCAM-1 scFv and IL22 can simultaneously bind to their intended targets.

Healthy human intestinal tissues were also incubated with biotinylated hMAdCAM-1 7A2scFv-IL22 to confirm that the bispecific does recognize hMAdCAM-1 when expressed on endothelial cells of the intestinal tract. Tissue sections showed a more diffuse staining pattern than the parent antibody, possibly due to the IL22 detecting its cognate receptor on the same tissue (FIG. 4E). Finally, the COLO205 cells also had elevated IL-10 secretion in the presence of 7A2scFv-IL22 compared to the 7A2 parent mAb, suggesting that the IL22 domain was functional (FIG. 4F). A hMAdCAM-1 scFv 7A2scFv-IL18 bp construct is currently undergoing similar development (data not shown). At present, the pharmacology and efficacy of the human-specific 7A2scFv-IL22 bispecific still needs to be evaluated in vivo in humans. To further validate the concept, we created equivalent mouse-specific constructs, to assess the in vivo targeting potential of MAdCAM-1 targeting bispecifics.

### Reference Cited

66. Chen, X., Zaro, J. L. & Shen, W. C. Fusion Protein Linkers: Property, Design and Functionality. Adv Drug Deliv Rev 65, 1357 (2013).

### Overall Discussion:

In the examples provided herein, we generated anti-human MAdCAM-1 antibodies to provide a basis for targeted human bispecifics (FIG. 1). These antibodies demonstrated binding to recombinant human MAdCAM-1 by surface plasmon resonance (FIG. 2A), when expressed on a cell surface (FIG. 2C), and on human small intestinal tissue (FIG. 2D). Moreover, the addition of clones 1B10, 11C3, 2F3, and 7A2 blocked MAdCAM-1 co-stimulation as evidenced by a significant reduction in the proliferation of the CD4+ T cells (FIG. 3A); in addition, it was found that the addition said clones favoured the expansion of CD45RA⁻CD27⁻similar to that observed for anti-CD3 stimulation alone (FIG. 3B). Based on this data, the mAb 7A2 was chosen to derive a scFv and create bispecific constructs where this hMAdCAM-1 scFv was fused to a therapeutic cytokine (IL22) known to reduce pro-inflammatory responses (FIG. 4A-C). The bispecific, 7A2scFv-IL22, bound simultaneously to hMAdCAM-1 and human IL22Rα (FIG. 4D), stained small intestinal tissue as shown by immunohistochemistry (FIG. 4E), and retained the IL22 functional activity (FIG. 4F). Since our hMAdCAM-1 mAbs did not cross-react with murine MAdCAM-1, we could not further assess their in vivo activity in IBD mouse models.

IL18 is known to induce the production of proinflammatory cytokines such as IFN, TNFα, and IL-6 in the colon. The impact of MAdCAM-1-targeted-IL18 bp bispecifics can be compared to untargeted IL 18 bp in terms of reducing the levels of proinflammatory cytokines295,296. IL 18 bp targets an inflammatory cytokine for which the expression is induced during inflammation.

Mechanistically, IL22 can induce STAT3 signaling in endothelial cells, where STAT3 activation has demonstrated anti-inflammatory mechanisms in different injury and inflammation models through role in IFN suppression³²⁹, the regulation of adhesion molecule expression³³⁰, and limiting leukocyte trafficking³³¹.

The strategy of MAdCAM-1 targeting could be applied to alternate therapeutic cargo such as, for example IL-23 blockers (ustekinumab, risankizumab) which may benefit from tethering to an anti-MAdCAM-1.

The small non-Ig-like linear bispecifics such as the ones described here, lack the Fc and are cleared from circulation within hours. Smaller bispecifics can be beneficial depending on the therapeutic cargo that is being used if the long-term accumulation or circulation poses a safety risk. This would allow for the therapeutic to rapidly reach the target tissue, improve local efficacy, then removed from circulation. A faster clearing scFv-linked IL22 that is “short-acting” compared to the “long-acting” IL22-Fc may thus provide a reduction in adverse effects, such as those related to the skin, as explored in the design of pancreas/liver-targeted IL22 constructs²⁹³. Although not examined in these examples, it is contemplated that extending the half-life of MAdCAM-1-targeted anti-inflammatoires could be beneficial, and as such, the use of full length antibodies or constructs comprising a silenced Fc domain that allows for an extended half-life (through its FcRn binding function) but without Fc-meditated effector functions (via Fcγ receptor and C1q binding) to avoid undesirable immune cell activation events, such as antibody-dependent cellular cytotoxicity against the MAdCAM-1-expressing cells, are also contemplated herein. An issue with extending the half-life of our bispecifics, however, is that most of their targeted anti-inflammatory cargos may remain in circulation causing systemic side effects.

Overall, we produced gut targeting bispecifics which may be a targeting strategy to accumulate anti-inflammatory therapeutics along the intestinal tract where inflamed sites are located and to reduce systemic effects associated with circulating anti-inflammatory cytokines.

### References Cited

295. Guo, L. et al. Production of recombinant human long-acting IL-18 binding protein: inhibitory effect on ulcerative colitis in mice. Appl Microbiol Biotechnol 107, 7135-7150 (2023).
296. Sivakumar, P. V et al. Interleukin 18 is a primary mediator of the inflammation associated with dextran sulphate sodium induced colitis: blocking interleukin 18 attenuates intestinal damage. Gut 50, 812-820 (2002).
329. Kano, A. et al. Endothelial Cells Require STAT3 for Protection against Endotoxin-induced Inf lammation. J Exp Med 198, 1517 (2003).
330. Wei, Z. et al. The IL-6/STAT3 pathway regulates adhesion molecules and cytoskeleton of endothelial cells in thromboangiitis obliterans. Cell Signal 44, 118-126 (2018).
331. Dube, S. et al. Endothelial STAT3 Modulates Protective Mechanisms in a Mouse Ischemia-Reperfusion Model of Acute Kidney Injury. J Immunol Res 2017, U.S. Pat. No. 4,609,502 (2017).
293. Sajiir, H. et al. Liver and pancreatic-targeted interleukin-22 as a therapeutic for metabolic dysfunction-associated steatohepatitis. Nature Communications 2024 15:1 15, 1-13 (2024).

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Without further description, it is believed that one of ordinary skill in the art can, using the preceding description and the following illustrative examples, make and utilize the constructs of the present invention and practice the claimed methods. The following working examples therefore, specifically point out the typical aspects of the present invention and are not to be construed as limiting in any way in the remainder of the disclosure. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. An antibody capable of binding to a gastrointestinal tract (GI) tract expressed antigen.

2. The antibody of claim 1, comprising or consisting of a polypeptide having the amino acid sequence of heavy chain complementarity-determining regions (HDCRs): HCDR1, HCDR2 and/or HCDR3 of clones 11C3, 15D6, 7A2, 2F3, 1B10, 13C5, 15A3, or 6A3, and a polypeptide having an amino acid sequence of light chain complementarity-determining regions (LCDRs): LCDR1, LCDR2 and/or LCDR3 of clones 11C3, 15D6, 7A2, 2F3, 1B10, 13C5, 15A3, or 6A3, wherein HCDR1, HCDR2, and HCDR3 are selected from:
(a) the sequence set forth in SEQ ID NO: 1, 3, and FAY of 11C3;
(b) the sequence set forth SEQ ID NO: 6, 8, and 10 of 15D6;
(c) the sequence set forth SEQ ID NO: 12, 14, and 16 of 7A2;
(d) the sequence set forth SEQ ID NO: 18, 20, and 22 of 2F3;
(e) the sequence set forth SEQ ID NO: 24, 26, and 28 of 1B10;
(f) the sequence set forth SEQ ID NO: 29, 32, and 34 of 13C5;
(g) the sequence set forth SEQ ID NO: 36, 38, and 40 of 15A3; and
(h) the sequence set forth SEQ ID NO: 42, 44, and 46 of 6A3;
and wherein LCDR1, LCDR2, and LCDR3 are selected from:
(i) the sequence set forth in SEQ ID NO: 2, 4, and 5 of 11C3;
(j) the sequence set forth SEQ ID NO: 7, 9, and 11 of 15D6;
(k) the sequence set forth SEQ ID NO: 13, 15, and 17 of 7A2;
(l) the sequence set forth SEQ ID NO: 19, 21, and 23 of 2F3;
(m) the sequence set forth SEQ ID NO: 25, 27, and 27 of 1B10;
(n) the sequence set forth SEQ ID NO: 31, 33, and 35 of 13C5;
(o) the sequence set forth SEQ ID NO: 37, 39, and 41 of 15A3; and
(p) the sequence set forth SEQ ID NO: 43, 45, and 47 of 6A3;
or functional variant that is capable of binding to the GI tract expressed antigen.

3. The antibody of claim 1, comprising or consisting of a polypeptide having the amino acid sequence of the heavy chain and a polypeptide having the amino acid sequence of the light chain of 11C3, 15D6, 7A2, 2F3, 1B10, 13C5, 15A3, or 6A3:
11C3 HEAVY CHAIN
(SEQ ID NO: 48)
MLLGLKWVFFVVFYQGVHCEVKLVESGGGLVQPKGSLKLSCAASGFTFNTYAMNWIR
QAPGKGLEWVARIRSKNNYYATYYADSVKDRFTISRDDSQSMLYLQMNNLKTEDTAM
YYCVSFAYWGQGTLVTVSA
11C3 LIGHT CHAIN
(SEQ ID NO: 49)
MRFSAQLLGLLVLWIPGSTAEIVMTQAAFSNPVTLGTSASISCRSSKSLLHSNGITYLYW
YLQKPGQSPRFLIYQMSNLASGVPDRFTSSGSGTDFTLRISRVEAEDVGVYYCAQNLELP
WTFGGGTKLEIK
15D6 HEAVY CHAIN
(SEQ ID NO: 50)
MDSRLNLVFLVLILKGVQCDVQLVESGGGLVQPGGSRKLSCAASGFTFSTFGMHWVRQ
APEKGLEWVAYISSGSSSIYYADTVKGRFTISRDNPVNTLFLQMTSLRSEDTAMYYCARS
GLGTSPHAMDYWGQGTSVTVSS
15D6 LIGHT CHAIN
(SEQ ID NO: 51)
MDFQVQIFSFLLMSASVIMSRGQIVLTQSPTLMSASPGEKVTMTCSASSSVGYMYWYQQ
KPRSSPKPWIYLTSILASGVPARFSGSGSGTSYSLTISSMEAEDAATYYCQQWISNPLTFG
AGTKLELK
7A2 HEAVY CHAIN
(SEQ ID NO: 52)
MAVLVLFLCLAAFPSYVLSQVQMEQSGPGLVAPSQSLSITCTVSDFSLINYGVHWVRQS
PGKGLEWLGVIWTGGNTEYNSALMSRLSITKDNSKSQVFLKMNSLQADDTAMYYCAR
DDYYFGGGAMDYWGQGTSVTVSS
7A2 LIGHT CHAIN
(SEQ ID NO: 53)
MVSTPQFLVFLLFWIPASRGDILLTQSPAILSVSPGERVSFACRASQTIGTNIHWYQQRTN
GSPRLLIKYASESISEIPSRFSGSGSGTDFILTISSVESEDIADYYCQQSNNWPLTFGAGTKL
ELR
2F3 HEAVY CHAIN
(SEQ ID NO: 54)
MEWRWIFLLLLSGTTGVHSEIQLQQSGPELVKPGASVKVSCKASGYTFTSFTLYWVKQS
HGKSLEWIGYIDPSNGGTTYNQRFKGKATLTVDKSSTTAYMHLNSLTSEDSSVYYCARG
IYDGYYVGKIFDYWGQGTTLTVSS
2F3 LIGHT CHAIN
(SEQ ID NO: 55)
METDTLLLWVLLLWVPGSTGNIVLTQSPTSLAVSLGQRATISCRASESVDNYGNSFMHW
YQQKPGQPPRLLIYLASNLESGVPARFSGSGSRTDFTLTIDPVEADDAATYYCQQNNEDP
PTFGGGTKLEIR
1B10 HEAVY CHAIN
(SEQ ID NO: 56)
MGWSYIILFLVATATDVHSQVQLQQPGAELVKPGASVKLSCKASGYTFTSYWMDWVK
QRPGQGLEWIGEIYPSNGRTKYNEKFKNKATLTVDNSSRTAYMHLSSLTSEDSAVYYCA
RGGYDVYYVGNTLDYWGQGTSVTVSS
1B10 LIGHT CHAIN
(SEQ ID NO: 57)
METDTLLLWVLLLWVPGSTGNIVLTQSPASLAVSLGQRATISCRASESIDSYGNSFMHW
YHQKPGQPPKLLIYLASNLESGVPARFSGSGSRTDFTLTIDPVEADDAATYYCLQNNGDP
WTFGGGTRLEIK
13C5 HEAVY CHAIN
(SEQ ID NO: 58)
MECNWILPFILSVTSGVYSEVQLQQSGTVLARPGASVKMSCKASGYTFTSYWMHWVK
QRPGQGLEWIGAIYPGNSDTSYNQKFKGKAKLTAVTSTSTAYMELSSLTNEDSAVYYCT
IYGNYGYFDVWGAGTTVTVSS
13C5 LIGHT CHAIN
(SEQ ID NO: 59)
MDSQAQVLMLLLLWVSGTCGDIVMSQSPSSLAVSVGEKVTMSCKSSQSLLYSSNQKNY
LAWYQQKPGQSPKLLIYWASTRESGVPDRFTGSGSGTDFTLTISSVKAEDLAVYYCQQY
YSYPPYTFGGGTKLEIK
15A3 HEAVY CHAIN
(SEQ ID NO: 60)
MEWRIFLFILSGTACVHSQDQLLQSGPELVKPGTSVKMSCRASGYTFSDDVINWVRKRT
GQGLEWIGEIYPRIGSMYYNENFKGRATLTADKSSNTVYIHLSSLTSEDSAVYFCARYLL
TKGYGMDYWGQGTSVTVSS
15A3 LIGHT CHAIN
(SEQ ID NO: 61)
METDTLLLWVLLLWVPGSTGDIVLTQSPASLAVSLGQRATISCRASKSVSPSDYSYIHW
YQQKPGQPPKLLIYLASNLESGVPARFSGSGSGTDFTLNIHPVEEEDAATYYCQHSRELP
RTFGGGTKLEIK
6A3 HEAVY CHAIN
(SEQ ID NO: 62)
MAVLGLLFCLVTFPSCVLSQVQLKQSGPGLVQPSQSLSITCTVSGFSLTSYGLHWVRQSP
GKGLEWLGFIWSGGATDYNAAFISRLSISKDNSKRQVFFKMNSLQANDTAIYFCARRDD
NYALAMDYWGQGTSVTVSS
6A3 LIGHT CHAIN
(SEQ ID NO: 63)
MDFQVQIFSFLLMSASVIMSRGQIVLTQSPALMSASPGEKVTMTCRASSSVSYIYWYQQ
KPRSSPKPWIYLTSDLASGVPTRFSGSGSGTSYFLTISSMEAEDTATYYCQQWNSNPLTF
GAGTKLELK
or a functional variant or fragment thereof that is capable of binding to the GI tract expressed antigen.

4. The antibody of claim 2, wherein the functional variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the recited amino acid sequence, or
wherein the antibody comprises or consists of at 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the recited amino acid sequence.

5. (canceled)

6. The antibody of claim 1, wherein the antibody:
(a) is selected from IgG, IgM, IgD, IgA, or IgD;
(b) is an IgG antibody having an isotype selected from IgG₁, IgG₂, IgG₃ or IgG₄;
(c) is a monoclonal antibody; and/or;
(d) binds to the GI tract expressed antigen; and/or
(e) has a K_{D} of between about 0.05 nM to about 20 nM for the GI tract expressed antigen.

7-10. (canceled)

11. The antibody of claim 1, wherein;
(a) the GI tract expressed antigen is Mucosal Addressin-Cell Adhesion Molecule 1 (MAdCAM-1); and/or
(b) the antibody binds to human MAdCAM-1.

12. The antibody of claim 1, wherein binding to the GI tract expressed antigen (a) targets the antibody to the GI tract and/or (b) preferentially expands T cells.

13. The antibody of claim 12, wherein the T cells are CD4+ and/or CD8+ T cells; optionally wherein the T cells are CD45⁻CD27⁻ T cells.

14-15. (canceled)

16. The antibody of claim 1, wherein the antibody is an anti-MAdCAM-1 antibody.

17. The antibody of claim 16, wherein the anti-MAdCAM-1 antibody is a monoclonal anti-MAdCAM-1 antibody and/or wherein the anti-MAdCAM-1 antibody is a human monoclonal anti-MAdCAM-1 antibody.

18. (canceled)

19. A construct comprising an antibody and a cargo molecule, wherein the antibody is capable of binding a gastrointestinal (GI) tract expressed antigen.

20. The construct of claim 19, wherein the antibody is a functional antibody fragment that binds to the GI tract expressed antigen and/or wherein the antibody is a functional antibody fragment that is fused to the cargo molecule and/or wherein the cargo molecule does not impact or impair the ability of the functional antibody fragment to bind to the GI tract expressed antigen.

21-22. (canceled)

23. The construct of claim 20, wherein the functional antibody fragment:
(a) has a K_{D} of between about 0.05 nM to about 20 nM for the GI tract expressed antigen;
(b) is selected from a scFv, a Fab′, a Fab, a F(ab′) 2, a Fv, or a scFab;
(c) is a scFv; and/or
(d) is a scFv comprising or consisting of a polypeptide having the sequence:
(SEQ ID NO: 128)
QVQMEQSGPGLVAPSQSLSITCTVSDFSLINYGVHWVRQSPGKGLEWLGV
IWTGGNTEYNSALMSRLSITKDNSKSQVFLKMNSLQADDTAMYYCARDDY
YFGGGAMDYWGQGTSVTVSSGGGGSGGGGSGGGGSDILLTQSPAILSVSP
GERVSFACRASQTIGTNIHWYQQRTNGSPRLLIKYASESISEIPSRFSGS
GSGTDFILTISSVESEDIADYYCQQSNNWPLTFGAGTKLELRAAAYPYDV
PDYAHHHHHH;
and/or
(e) is a scFv capable of binding, or binds to, MAdCAM1.

24-26. (canceled)

27. The construct of claim 23, further comprises a secretion sequence, as set forth in SEQ ID NO: 130, at the N-terminus of sequence.

28. The construct of claim 19, wherein binding to the GI tract expressed antigen (a) targets the construct to the GI tract and/or (b) preferentially expands T cells.

29. The construct of claim 28, wherein the T cells are CD4+ and/or CD8+ T cells; optionally wherein the T cells are CD45 CD27 T cells.

30. (canceled)

31. The construct of claim 19, wherein the GI tract expressed antigen is Mucosal Addressin-Cell Adhesion Molecule 1 (MAdCAM-1); optionally wherein the GI tract expressed antigen is expressed in the small intestine, the large intestine and/or the colon.

32. (canceled)

33. The construct of claim 19, wherein the cargo molecule is:
(a) selected from proteins, peptides, nucleic acids, amino acids, nucleosides, antibodies, antibody fragments, antibody ligands, peptide nucleic acids, small organic molecules, lipids, hormones, drugs, enzymes, lectin, cell adhesion molecule, antibody epitope, enzyme substrates, enzyme inhibitors, coenzymes, inorganic molecules, carbohydrates, such as polysaccharides and monosaccharides, or a combination thereof;
(b) a protein; and/or
(c) IL-22, IL-18 bp or an IL-23 blocker such as Ustekinumab, Risankizumab.

34-35. (canceled)

36. The construct of claim 19, wherein when the construct is targeted to the GI tract expressed antigen, the construct is capable of limiting off-target effects of the cargo molecule and/or wherein when the construct is targeted to the GI tract expressed antigen, the construct can allow for accumulation of the cargo molecule at a targeted site in the GI tract.

37-38. (canceled)

39. The construct of claim 19, wherein at least one of the following is true:
(a) the construct allows for inhibition of IL-18; (b) the construct allows for production of IL-10; (c) the construct induces STAT3 phosphorylation; (d) the construct reduces IFNγ secretion.

40. The construct of claim 19 having the sequence:
(SEQ ID NO: 132)
ATMETDTLLLWVLLLWVPGSTGQVQMEQSGPGLVAPSQSLSITCTVSDFS
LINYGVHWVRQSPGKGLEWLGVIWTGGNTEYNSALMSRLSITKDNSKSQV
FLKMNSLQADDTAMYYCARDDYYFGGGAMDYWGQGTSVTVSSGGGGSGGG
GSGGGGSDILLTQSPAILSVSPGERVSFACRASQTIGTNIHWYQQRTNGS
PRLLIKYASESISEIPSRFSGSGSGTDFILTISSVESEDIADYYCQQSNN
WPLTFGAGTKLELRGGGGSGGGGSGGGGSGGGGSGGGGSAPISSHCRLDK
SNFQQPYITNRTFMLAKEASLADNNTDVRLIGEKLFHGVSMSERCYLMKQ
VLNFTLEEVLFPQSDRFQPYMQEVVPFLARLSNRLSTCHIEGDDLHIQRN
VQKLKDTVKKLGESGEIKAIGELDLLFMSLRNACIAAAYPYDVPDYAHHH
HHH.

41. (canceled)

42. A polynucleotide encoding the construct of claim 19, having the sequence of
(SEQ ID NO: 134)
GCCACCATGGAAACCGACACACTGCTGCTGTGGGTGCTGCTTCTTTGGGT
GCCCGGATCTACAGGCCAGGTGCAGATGGAACAGTCTGGCCCTGGACTGG
TGGCCCCATCTCAGAGCCTGAGCATCACCTGTACCGTGTCCGACTTCAGC
CTGATCAACTACGGCGTGCACTGGGTCCGACAGAGCCCTGGAAAAGGACT
GGAATGGCTGGGCGTGATCTGGACCGGCGGCAACACAGAGTACAACAGCG
CCCTGATGAGCCGGCTGTCCATCACCAAGGACAACAGCAAGAGCCAGGTG
TTCCTGAAGATGAACTCCCTGCAGGCCGACGACACCGCCATGTACTACTG
CGCCAGAGATGACTACTACTTCGGCGGAGGCGCCATGGATTATTGGGGCC
AGGGAACAAGCGTGACCGTGTCTAGCGGAGGCGGAGGATCTGGTGGCGGA
GGTAGTGGTGGCGGCGGATCTGATATTCTGCTGACACAGTCCCCTGCCAT
CCTGTCCGTTTCTCCAGGCGAGAGAGTGTCCTTCGCCTGTAGAGCCTCTC
AGACCATCGGCACCAACATCCACTGGTATCAGCAGCGGACCAACGGCAGC
CCCAGACTGCTGATTAAGTACGCCAGCGAGAGCATCAGCGAGATCCCCAG
CAGATTTTCTGGCAGCGGCTCCGGCACCGATTTCATCCTGACAATCAGCT
CCGTGGAAAGCGAGGATATCGCCGATTACTACTGCCAGCAGAGCAACAAC
TGGCCCCTGACATTTGGAGCCGGCACCAAGCTGGAACTTAGAGGCGGCGG
AGGTTCTGGCGGTGGTGGATCTGGCGGAGGTGGAAGCGGCGGAGGCGGCT
CTGGCGGCGGAGGAAGTGCTCCTATTAGCAGCCACTGCCGGCTGGACAAG
AGCAACTTCCAGCAGCCTTACATCACCAACCGGACCTTCATGCTGGCCAA
AGAGGCCAGCCTGGCCGACAACAATACTGACGTGCGGCTGATCGGCGAGA
AGCTGTTTCATGGCGTGTCCATGAGCGAGCGGTGCTACCTGATGAAGCAG
GTCCTGAACTTCACCCTGGAAGAGGTGCTGTTCCCTCAGAGCGACCGGTT
TCAGCCCTACATGCAAGAGGTGGTGCCCTTTCTGGCCCGGCTGAGCAATA
GACTGAGCACCTGTCACATCGAGGGCGACGACCTGCACATCCAGAGAAAC
GTGCAGAAACTGAAGGACACCGTGAAGAAGCTGGGCGAGAGCGGAGAGAT
CAAGGCCATCGGAGAACTGGACCTGCTGTTCATGAGCCTGCGGAACGCCT
GTATCGCCGCTGCCTATCCTTACGACGTGCCCGATTATGCCCACCACCAC
CATCACCACTGATGATGA.

43-52. (canceled)

53. A method of targeting the construct of claim 19 to a GI tract expressed antigen, the method comprising administering or the construct to a subject and targeting the construct to the GI tract of the subject.

54. (canceled)

55. The method of claim 53, wherein the GI tract expressed antigen is Mucosal Addressin-Cell Adhesion Molecule 1 (MAdCAM-1).

56. The method of claim 53, further comprising: (a) delivering the construct to the GI tract and/or (b) allowing accumulation of the cargo molecule in a targeted site in the GI tract, optionally wherein the targeted site in the GI tract comprises the small intestine, the large intestine and/or the colon.

57. (canceled)

58. A method of expanding T cells, the method comprising binding the construct of claim 19 to a gastrointestinal (GI) tract expressed antigen, thereby expanding the T cells.

59. The method of claim 58, wherein the T cells are CD4+ and/or CD8+ T cells, optionally, wherein the T cells are CD45⁻CD27⁻ T cells.

60-63. (canceled)
